SEQUENCE LISTING (13 sequences)

SEQ ID NO: 1 - DNA, 200 bp, Homo sapiens
aggtcctcatagtggagacgtgctgataataaattcactcccagaaaaaaagtccccatcctgattatttccctaattagcactggaaggtcaaattaagggaaaaaatgtatacacacacacacacacacacacacacacacacacacacatcctaccaaatcatacctttaactaggagtttacctcctaggcatgcc

SEQ ID NO: 2 - DNA, 20 bp, Homo sapiens
aggtcctcatagtggagacg

SEQ ID NO: 3 - DNA, 20 bp, Homo sapiens
atgcctaggaggtaaactcc

SEQ ID NO: 4 - DNA, 348 bp, Homo sapiens
aataacctaaaatcctaaatgtaattagcatgctggcattgaaaacaatcttgtaaataaataagtaatgatacagaatgatatcgacaatggattgttaggtgaaaaagataggctcaaaaacaatatgctgtatagatttcctgaatatatgtttacacacacacacacacacacacacacacacacacacacacacacacacacacggaagagacatattggaggctaatagcatttaacagtggttttctttgattgggaggattatgtgtaattttaattttctttgtttcgttcacttgttttgtctatttggggactgctatttttgctttaaaaattgca

SEQ ID NO: 5 - DNA, 20 bp, Homo sapiens
ggtgaaaaagataggctcaa

SEQ ID NO: 6 - DNA, 22 bp, Homo sapiens
ccactgttaaatgctattagcc

SEQ ID NO: 7 - DNA, 469 bp, Homo sapiens
gttctgtcatagctccatttcactaataaggagacagatgtggaggttggggagttggtcccaggtcacccaactggggagggcagaggttggggagggacaggagtcaataacccaaagtcatgaaatgagaaaggaagtaaacacttggatggagaatcacacacacacacacacacacacacacacacacacacacacacacacacctcctaacaggtatgttgtctgcaacaaggcaaaaataattcattaatatctcatttaaacttgagggcgagggaattcctgaaccacctctctggagcaaataatggaaattggaaattgattgtcatttacctttgaggaagacttcgggatgtgccatgtctttggtatagggctgcgtggtgttgtgacgcatgtgaagaaatacatccaaggaccttcctaagctcatctgcagccacaattcccccaccctatt

SEQ ID NO: 8 - DNA, 20 bp, Homo sapiens
cccaaagtcatgaaatgaga

SEQ ID NO: 9 - DNA, 22 bp, Homo sapiens
acaacatacctgttaggaggtg

SEQ ID NO: 10 - DNA, 386 bp, Homo sapiens
  misc_feature (142)..(142): n is a, c, g, or t
agctccatttcactaataaggagacagatgtggaggttggggagttggtcccaggtcacccaactggggagggcagaggttggggagggacaggagtcaataacccaaagtcatgaaatgagaaaggaagtaaacacttggntggagantcacacacacacacacacacacacacacacacacacacctcctaacaggtatgttgtctgcaacaaggcaaaaataattcattaatatctcatttaaacttgagggcgagggaattcctgaaccacctctctggagcaaataatggaaattggaaattgattgtcatttacctttgaggaagacttcgggatgtgccatgtctttggtatagggctgcgtggtgttgtgacgcatgt

SEQ ID NO: 11 - DNA, 324 bp, Homo sapiens
atttctatgacttagatattctgcatcacaaaatccctccaaactgggactatgtttttgaagtcattcattttacaattataacaacaataacaataatatttattgtttgctttgtgccaggtactctactgctttacataaattatctcattctgtcacatctaacggcaactaagtatacgcttacatctgctagtggcacctaaaataaggatattgttggtcatctttaaagaaatgtcttaacataccaaagtagtggaatcatagaataaaatatttaagtcttacaaagcgtacgacactaaagtaatataggat

SEQ ID NO: 12 - DNA, 22 bp, Homo sapiens
atttattgtttgctttgtgcca

SEQ ID NO: 13 - DNA, 25 bp, Homo sapiens
caacaatatccttattttaggtgcc

(19) United States
(12) Patent Application Publication
(10) Pub. No.: US 2013/0225420 A1
(43) Pub. Date: Aug. 29, 2013

(54) MOLECULAR SUBTYPING OF ORAL SQUAMOUS CELL CARCINOMA TO DISTINGUISH A SUBTYPE THAT IS UNLIKELY TO METASTASIZE

(71) Applicants: Donna G. Albertson, Lafayette, CA (US); Brian L. Schmidt, San Francisco, CA (US); Aditi Bhattacharya, Dallas, TX (US); Adam B. Olshen, Palo Alto, CA (US)

(72) Inventors: Donna G. Albertson, Lafayette, CA (US); Brian L. Schmidt, San Francisco, CA (US); Aditi Bhattacharya, Dallas, TX (US); Adam B. Olshen, Palo Alto, CA (US)

(73) Assignee: THE REGENTS OF THE UNIVERSITY OF CALIFORNIA, Oakland, CA (US)

(21) Appl. No.: 13/812,836

(22) PCT Filed: Aug. 2, 2011
(86) PCT No.: PCT/US11/01377

§ 371 Date: May 13, 2013

Related U.S. Application Data
(60) Provisional application No. 61/400,813, filed on Aug. 2, 2010.

(51) Int. Cl. C12Q 1/68

(52) U.S. Cl. CPC C12Q 1/6886
USPC 506/2; 435/6.11; 506/9

(57) ABSTRACT

The present invention provides methods of analyzing a sample from a subject having oral epithelial dysplasia or oral SCC or suspected of having oral epithelial dysplasia or oral SCC.

### CROSS-REFERENCE TO RELATED APPLICATIONS

This application claims the benefit of U.S. Provisional Application No. 61/400,813, filed on Aug. 2, 2010, the entire disclosure of which is hereby incorporated herein by reference for all purposes.

### STATEMENT AS TO RIGHTS TO INVENTIONS MADE UNDER FEDERALLY SPONSORED RESEARCH AND DEVELOPMENT

This invention was made with government support under grant nos. R01CA90421, R01CA113833, R01CA118323, R01CA131286, and R33CA94407 awarded by the National Institutes of Health. The Government has certain rights in the invention.

### FIELD OF THE INVENTION

The present invention relates generally to the area of molecular subtyping of cancer to distinguish a subtype this is unlikely to metastasize.

### BACKGROUND OF THE INVENTION

The 5-year survival rate for patients with oral squamous cell carcinoma (SCC), at 40%, is among the worst of all sites in the body and has not improved over the past 40 years. In the United States, more people die from oral cancer than melanoma, cervical cancer, or ovarian cancer. For patients with oral SCC, neck (cervical) metastasis is the primary determinant for prognosis, and once the neck lymph nodes are involved, the survival rate is reduced by one-half. Treatment for oral cancer is primarily surgical. Patients are assessed prior to surgery for lymph node metastasis by palpation of the lymph nodes in the neck and by imaging (CT, MRI, PET scan). If the neck is clinically positive, the treatment decision is straightforward, and the cervical lymph nodes and associated structures are removed during surgical resection of the tumor. Management of patients with clinically negative (N0) necks is less clear, given the unpredictable propensity of oral SCC for occult neck metastasis and the associated grave prognosis. Occult metastatic rates for oral SCC are high and range from 20-45% for T1 tongue SCCs. Treatment options include a “wait and see” approach and elective neck dissection. On the one hand, salvage rates of patients developing neck metastasis following the initial surgery are poor, while on the other hand, elective neck dissection may subject the patient to unnecessary major surgery with its associated risks and morbidity. Currently, tumor thickness is considered the best predictor of metastasis; however, it is difficult to assess this parameter from the incisional biopsy prior to surgery. Thus, the current standard of care is the American Joint Commission on Cancer (AJCC) TNM staging protocol, which is based on the surface diameter of the tumor.

There are currently no reliable molecular biomarkers for discriminating patients with and without oral SCC metastases prior to surgery.

### SUMMARY OF THE INVENTION

In some embodiments, the invention provides a first method of determining the presence of oral squamous cell carcinoma that is unlikely to metastasize by analyzing a biological sample, e.g., an oral sample, from a subject. In various embodiments, the method entails determining relative copy numbers in sample DNA for the following chromosomal regions: 3q, 8p, 8q, and 20, wherein no gain of chromosomal regions 3q, 8q, and 20, and no loss of chromosomal region 8p is indicative of oral squamous cell carcinoma that is unlikely to metastasize. In various embodiments, the method entails determining relative copy numbers in sample DNA for the following chromosomal regions: 3q, 8p, 8q, and 20, wherein a gain of one or more (e.g., two or three) of chromosomal regions 3q, 8q, and 20, and/or a loss of chromosomal region 8p is indicative of oral squamous cell carcinoma having a substantial likelihood of metastasis. In certain embodiments, the method entails determining relative copy numbers in sample DNA for the following chromosomal regions: 3q24-qter, 8pter-p23.1, 8q12-q24.2, and 20pter-qter, wherein no gain of chromosomal regions 3q24-qter, 8q12-q24.2, and 20pter-qter and no loss of chromosomal region 8pter-p23.1 is indicative of oral squamous cell carcinoma that is unlikely to metastasize. In certain embodiments, the method entails determining relative copy numbers in sample DNA for the following chromosomal regions: 3q24-qter, 8pter-p23.1, 8q12-q24.2, and 20pter-qter, wherein a gain of one or more (e.g., two or three) of chromosomal regions 3q24-qter, 8q12-q24.2, and 20pter-qter and/or a loss of chromosomal region 8pter-p23.1 is indicative of oral squamous cell carcinoma having a substantial likelihood of metastasis.

In illustrative embodiments, chromosomal region 3q24-qter extends from SEQ ID NO:1 to the q terminus of chromosome 3, chromosomal region 8pter-p23.1 extends from the p terminus of chromosome 8 to SEQ ID NO:7, and chromosomal region 8q12-q24.2 extends from SEQ ID NO:11 to SEQ ID NO:4.

In illustrative embodiments, the first method is carried out by contacting sample DNA with a combination of probes for chromosomal regions 3q, 8p, 8q, and 20, incubating the probes with the sample under conditions in which each probe binds selectively with a nucleic acid sequence in its target chromosomal region to form a stable hybridization complex, and detecting hybridization of the probes to determine copy number for each chromosomal region. For example, the method can be carried out by hybridization of sample nucleic acids to said combination of probes, which are immobilized on a substrate. In some embodiments, the method is carried out by array comparative genomic hybridization (aCGH). The combination of probes can, in some embodiments, include a plurality of probes for each chromosomal region. In certain embodiments, the combination of probes includes a plurality of probes for each of one or more control chromosomal regions. In another embodiment, the method is carried out by in situ hybridization, and each probe in the probe combination is labeled with a different label. In some embodiments, the probe combination includes at least 4, but not more than about 10¹² probes, for example, not more than about 10¹¹ probes, 10¹⁰ probes, 10⁹ probes, 10⁸ probes, 10⁷ probes, 10⁶ probes, or 10⁵ probes. In some embodiments, the probe combination includes at least 4, but not more than 10,000 probes. In some embodiments, the probe combination includes at least 4, but not more than 1000 probes. In various embodiments, the probe combination includes at least 4, but not more than 100 probes. In particular embodiments, the probe combination includes at least 4, but not more than 10 probes.

In certain embodiments, the first method entails amplification of target nucleic acids in chromosomal regions 3q, 8p, 8q, and 20, for example, by polymerase chain reaction (PCR) or multiplex ligation-dependent probe amplification (MLPA). In some embodiments, the method includes producing a plurality of amplicons from a plurality of target nucleic acids in each chromosomal region. In certain embodiments, the method includes producing a plurality of amplicons from a plurality of target nucleic acids in each of one or more control chromosomal regions.

In particular embodiments, the first method entails high-throughput DNA sequencing. The method can, in some embodiments, include sequencing a plurality of target nucleic acids in each chromosomal region. In certain embodiments, the method includes sequencing a plurality of target nucleic acids in each of one or more control chromosomal regions.

In some embodiments, the invention provides a second method of determining the presence of oral squamous cell carcinoma that is unlikely to metastasize in an oral sample from a subject. The method entails determining fraction of genome gained, wherein if the fraction of genome gained is below 0.065, the oral squamous cell carcinoma is unlikely to metastasize. In some embodiments, the invention provides a second method of determining the presence of oral squamous cell carcinoma that has a substantial likelihood of metastasis in an oral sample from a subject. The method entails determining fraction of genome gained, wherein if the fraction of genome gained is greater than 0.065, the oral squamous cell carcinoma has a substantial likelihood of metastasis. In embodiments where it is determined that the oral SCC has a substantial likelihood of metastasis, the method can further comprise evaluating a lymph node sample, e.g., from a cervical lymph node. In particular embodiments, the method entails determining relative copy numbers for a plurality of target nucleic acids.

In some embodiments, the invention provides a third method of determining the presence of oral squamous cell carcinoma that is unlikely to metastasize in an oral sample from a subject. The method entails determining fraction of genome altered, wherein if the fraction of genome altered is below 0.095, the oral squamous cell carcinoma is unlikely to metastasize. In some embodiments, the invention provides a third method of determining the presence of oral squamous cell carcinoma that has a substantial likelihood of metastasis in an oral sample from a subject. The method entails determining fraction of genome altered, wherein if the fraction of genome altered is greater than 0.095, the oral squamous cell carcinoma has a substantial likelihood of metastasis. In embodiments where it is determined that the oral SCC has a substantial likelihood of metastasis, the method can further comprise evaluating a lymph node sample, e.g., from a cervical lymph node. In particular embodiments, the method entails determining relative copy numbers for a plurality of target nucleic acids.

The second and third methods can, in certain embodiments, be carried out by hybridization of sample nucleic acids to a combination of probes, which are immobilized on a substrate, e.g., as in array comparative genomic hybridization (aCGH). In particular embodiments, the combination of probes can include a plurality of probes for each of one or more control chromosomal regions.

In certain embodiments, the second and third methods entail amplification of target nucleic acids, for example, by polymerase chain reaction (PCR) or multiplex ligation-dependent probe amplification (MLPA). In some embodiments, the methods include producing a plurality of amplicons from a plurality of target nucleic acids in each of one or more control chromosomal regions.

In particular embodiments, the second and third methods entail high-throughput DNA sequencing. The methods can, in some embodiments, include sequencing a plurality of target nucleic acids in each of one or more control chromosomal regions.

In any of the above-described embodiments, relative copy numbers can be determined by analyzing genomic DNA. In other embodiments, relative copy numbers can be determined by analyzing RNA, cDNA, or DNA amplified from RNA.

Any of the above-described methods can, in certain embodiments, additionally entail querying the copy number(s) of one or more control chromosomal regions.

In various embodiments, where there is an indication that the oral squamous cell carcinoma has a substantial likelihood of metastasis, the method can further comprise determining the presence of one or more genetic alterations selected from the group consisting of: fraction of genome gained (FGG), fraction of genome altered (FGA), altered methylation status, TP53 mutation(s), and the presence of relative copy number alterations at one or more loci other than 3q24-qter, 8pter-p23.1, 8q12-q24.2, and 20pter-qter, wherein the presence of one or more of said genetic alterations indicates an increased likelihood that metastasis will occur or has occurred. In various embodiments, where there is an indication that the oral squamous cell carcinoma has a substantial likelihood of metastasis, the method can further comprise determining one or more clinical parameters selected from the group consisting of tumor size, tumor thickness, tumor stage, the presence of metastasis (e.g., by radiographic imaging, and palpation of the neck).

In any of the above-described embodiments, the biological sample can include an oral sample, a sample of the primary tumor, and a sample at the margin of the tumor. In some embodiments, the biological sample is an oral sample. In any of the above-described embodiments, the oral sample can include saliva, an oral washing sample, an oral swab or brush sample, or an oral tissue sample from a site selected from the group consisting of: tongue, gingiva, floor of mouth, retromolar trigone, buccal mucosa, and lip.

If the results of any of these methods indicate the presence of oral squamous cell carcinoma that is unlikely to metastasize, the method can, in some embodiments, additionally include treating the subject for oral squamous cell carcinoma without removing the cervical lymph nodes. In various embodiments, when the results of the method indicates the presence of oral squamous cell carcinoma having a substantial likelihood of metastasis, the method additionally comprises determining relative copy numbers in sample DNA from one or more cervical lymph nodes for one or more (e.g., two, three or four) of the following chromosomal regions: 3q, 8p, 8q, and 20. In various embodiments, when the results of the method indicates the presence of oral squamous cell carcinoma having a substantial likelihood of metastasis, the method additionally comprises removing one or more cervical lymph nodes from the subject.

In a further aspect, the invention provides a method of assessing the risk, that if an oral epithelial dysplasia progresses, the oral epithelial dysplasia will progress to oral squamous cell carcinoma having a substantial likelihood of metastasis, the method comprising determining relative DNA copy numbers in a biological sample from a subject for the following chromosomal regions: 3q, 8p, 8q, and 20, wherein no gain of chromosomal regions 3q, 8q, and 20, and no loss of chromosomal region 8p is indicative of oral epithelial dysplasia that, if it progresses, is unlikely to progress to metastatic oral squamous cell carcinoma, and wherein a gain of one or more (e.g., two or three) of chromosomal regions 3q, 8q, and 20, and/or a loss of chromosomal region 8p is indicative of oral epithelial dysplasia that, if it progresses, will progress to oral squamous cell carcinoma having a substantial likelihood of metastasis.

In various embodiments, the method comprises determining relative copy numbers in sample DNA for the following chromosomal regions: 3q24-qter, 8pter-p23.1, 8q12-q24.2, and 20pter-qter, wherein no gain of chromosomal regions 3q24-qter, 8q12-q24.2, and 20pter-qter and no loss of chromosomal region 8pter-p23.1 is indicative of oral epithelial dysplasia that, if it progresses, is unlikely to progress to metastatic oral squamous cell carcinoma, and wherein a gain of one or more (e.g., two or three) chromosomal regions 3q24-qter, 8q12-q24.2, and 20pter-qter and/or a loss of chromosomal region 8pter-p23.1 is indicative of oral epithelial dysplasia that, if it progresses, will progress to oral squamous cell carcinoma having a substantial likelihood of metastasis.

In some embodiments, the method comprises additionally monitoring the oral dysplasia for evidence of progression to oral squamous cell carcinoma.

In some embodiments, if the biological sample has a gain or loss of one or more (e.g., two, three or four) of said chromosomal regions, the method further comprises determining the presence of one or more genetic alterations selected from the group consisting of: fraction of genome gained (FGG), fraction of genome altered (FGA), methylation status, TP53 mutation(s), and the presence of relative copy number alterations at one or more loci other than 3q24-qter, 8pter-p23.1, 8q12-q24.2, and 20pter-qter, wherein the presence of one or more of said genetic alterations indicates an increased risk that the oral epithelial dysplasia is progressing, has progressed, or has a substantial likelihood of progressing.

In some embodiments, if the biological sample has a gain or loss of one or more (e.g., two, three or four) of said chromosomal regions, the method further comprises determining the presence of relative copy number alterations at one or more (e.g., 2, 3, 4, 5, 6, 7, 8, 9, 10, 11, 12, 13, 14, 15 or all) loci selected from the group consisting of 3pter-p14.1, 4p15.3-p15.2, 4q33-4-q35, 5pter-p13.2, 5q12-q23, 7p11.2-p12.1, 8p23.3-p21.2, 8p12, 8q11.1-qter, 9pter-p21.1, 11q13-q13.4, 18q22-qter, 20pter-p13, 20p12.2 and 21q21.3, wherein the presence of one or more of said copy number alterations indicates an increased risk that the oral epithelial dysplasia is progressing, has progressed, or has a substantial likelihood of progressing.

In some embodiments, if the biological sample has a gain or loss of one or more (e.g., two, three or four) of said chromosomal regions, the method further comprises treating the oral dysplasia more aggressively than if the results of the method indicated that the oral dysplasia was unlikely to progress to metastatic oral squamous cell carcinoma.

In some embodiments of the method for assessing oral epithelial dysplasia, if the biological sample has a gain or loss of one or more (e.g., two, three or four) of said chromosomal regions, the method further comprises determining one or more clinical parameters selected from the group consisting of dysplasia grade, presence of erythroplakia, toluidine blue staining, presence of ulcer (i.e., ulcerated lesion), and pain.

In some embodiments of the method for assessing oral epithelial dysplasia, chromosomal region:

3q24-qter extends from SEQ ID NO:1 to the q terminus of chromosome 3;
8pter-p23.1 extends from the p terminus of chromosome 8 to SEQ ID NO:7; and
8q12-q24.2 extends from SEQ ID NO:11 to SEQ ID NO:4.

In various embodiments of the method for assessing oral epithelial dysplasia, the relative copy numbers are determined by analyzing genomic DNA. In various embodiments of the method for assessing oral epithelial dysplasia, the relative copy numbers are determined by analyzing RNA, cDNA, or DNA amplified from RNA. In various embodiments, the method for assessing oral epithelial dysplasia additionally comprises querying the copy number(s) of one or more control chromosomal regions.

In various embodiments of the method for assessing oral epithelial dysplasia, the method comprises:

contacting sample DNA with a combination of probes for chromosomal regions 3q, 8p, 8q, and 20;

incubating the probes with the sample under conditions in which each probe binds selectively with a nucleic acid sequence in its target chromosomal region to form a stable hybridization complex; and

detecting hybridization of the probes to determine copy number for each chromosomal region.

In some embodiments of the method for assessing oral epithelial dysplasia, the method is carried out by hybridization of sample nucleic acids to said combination of probes, which are immobilized on a substrate. In various embodiments, this method is carried out by array comparative genomic hybridization (aCGH). In various embodiments of the method for assessing oral epithelial dysplasia, the combination of probes comprises a plurality of probes for each chromosomal region. In various embodiments of this method, the combination of probes comprises a plurality of probes for each of one or more control chromosomal regions. In some embodiments, the probe combination includes at least 4, but not more than about 10¹² probes, for example, not more than about 10¹¹ probes, 10¹⁰ probes, 10⁹ probes, 10⁸ probes, 10⁷ probes, 10⁶ probes, or 10⁵ probes. In various embodiments of this method, the probe combination comprises at least 4, but not more than 10,000 probes. In various embodiments of this method, the probe combination comprises at least 4, but not more than 1000 probes. In various embodiments of this method, the probe combination comprises at least 4, but not more than 100 probes. In various embodiments of this method, the probe combination comprises at least 4, but not more than 10 probes.

In some embodiments of the method for assessing oral epithelial dysplasia, the method is carried out by in situ hybridization, and each probe in the probe combination is labeled with a different label. In various embodiments of this method, the probe combination comprises at least 4, but not more than 1000 probes. In various embodiments of this method, the probe combination comprises at least 4, but not more than 100 probes. In various embodiments of this method, the probe combination comprises at least 4, but not more than 10 probes.

In some embodiments of the method for assessing oral epithelial dysplasia, the method comprises amplification of target nucleic acids in chromosomal regions 3q, 8p, 8q, and 20. In various embodiments, this method comprises polymerase chain reaction (PCR) or multiplex ligation-dependent probe amplification (MLPA). In various embodiments, this method comprises producing a plurality of amplicons from a plurality of target nucleic acids in each chromosomal region. In various embodiments, this method comprises producing a plurality of amplicons from a plurality of target nucleic acids in each of one or more control chromosomal regions.

In some embodiments of the method for assessing oral epithelial dysplasia, the method comprises high-throughput DNA sequencing. In various embodiments, this method comprises sequencing a plurality of target nucleic acids in each chromosomal region. In various embodiments, this method comprises sequencing a plurality of target nucleic acids in each of one or more control chromosomal regions.

In any of the above-described embodiments of the method for assessing oral epithelial dysplasia, the biological sample can include an oral sample, a sample of the primary dysplasia, and a sample at the margin of the dysplasia. In some embodiments of this method, the biological sample is an oral sample. In some embodiments of this method, the oral sample comprises saliva, an oral washing sample, an oral swab or brush sample, or an oral tissue sample from a site selected from the group consisting of: tongue, gingiva, floor of mouth, retromolar trigone, buccal mucosa, and lip.

In some embodiments, when the results of the method indicate the oral dysplasia is likely to progress to metastatic oral squamous cell carcinoma, the method additionally comprises treating the oral dysplasia more aggressively than if the results of the method indicated that the oral dysplasia was unlikely to progress to metastatic oral squamous cell carcinoma.

In a related aspect, the invention provides a method of determining the presence of metastatic oral squamous cell carcinoma in a lymph node sample from a subject, the method comprising determining relative copy numbers in sample DNA for the following chromosomal regions: 3q, 8p, 8q, and 20, wherein a gain of one or more (e.g., two or three) of chromosomal regions 3q, 8q, and 20, and/or a loss of chromosomal region 8p is indicative of metastatic oral squamous cell carcinoma.

In some embodiments, the method of determining the presence of metastatic oral squamous cell carcinoma comprises determining relative copy numbers in sample DNA for the following chromosomal regions: 3q24-qter, 8pter-p23.1, 8q12-q24.2, and 20pter-qter, wherein a gain of one or more (e.g., two or three) of chromosomal regions 3q24-qter, 8q12-q24.2, and 20pter-qter and/or a loss of chromosomal region 8pter-p23.1 is indicative of metastatic oral squamous cell carcinoma.

In some embodiments of the method of determining the presence of metastatic oral squamous cell carcinoma, the method further comprises determining the presence of one or more genetic alterations selected from the group consisting of: fraction of genome gained (FGG), fraction of genome altered (FGA), methylation status, TP53 mutation(s), and the presence of relative copy number alterations at one or more loci other than 3q24-qter, 8pter-p23.1, 8q12-q24.2, and 20pter-qter.

In some embodiments of the method of determining the presence of metastatic oral squamous cell carcinoma, the chromosomal region:

3q24-qter extends from SEQ ID NO:1 to the q terminus of chromosome 3;

8pter-p23.1 extends from the p terminus of chromosome 8 to SEQ ID NO:7; and

8q12-q24.2 extends from SEQ ID NO:11 to SEQ ID NO:4.

In some embodiments of the method of determining the presence of metastatic oral squamous cell carcinoma, the relative copy numbers are determined by analyzing genomic DNA. In some embodiments, relative copy numbers are determined by analyzing RNA, cDNA, or DNA amplified from RNA.

In some embodiments of the method of determining the presence of metastatic oral squamous cell carcinoma, the method additionally comprises querying the copy number(s) of one or more control chromosomal regions.

In some embodiments of the method of determining the presence of metastatic oral squamous cell carcinoma, the method comprises:

contacting sample DNA with a combination of probes for chromosomal regions 3q, 8p, 8q, and 20;

incubating the probes with the sample under conditions in which each probe binds selectively with a nucleic acid sequence in its target chromosomal region to form a stable hybridization complex; and

detecting hybridization of the probes to determine copy number for each chromosomal region.

In some embodiments of the method of determining the presence of metastatic oral squamous cell carcinoma, the method is carried out by hybridization of sample nucleic acids to said combination of probes, which are immobilized on a substrate. In various embodiments, this method is carried out by array comparative genomic hybridization (aCGH). In various embodiments of this method, the combination of probes comprises a plurality of probes for each chromosomal region. In some embodiments of this method, the combination of probes comprises a plurality of probes for each of one or more control chromosomal regions. In some embodiments, the probe combination includes at least 4, but not more than about 10¹² probes, for example, not more than about 10¹¹ probes, 10¹⁰ probes, 10⁹ probes, 10⁸ probes, 10⁷ probes, 10⁶ probes, or 10⁵ probes. In some embodiments of this method, the probe combination comprises at least 4, but not more than 10,000 probes. In some embodiments of this method, the probe combination comprises at least 4, but not more than 1000 probes. In some embodiments of this method, the probe combination comprises at least 4, but not more than 100 probes. In some embodiments of this method, the probe combination comprises at least 4, but not more than 10 probes.

In some embodiments of the method of determining the presence of metastatic oral squamous cell carcinoma, the method is carried out by in situ hybridization, and each probe in the probe combination is labeled with a different label. In some embodiments of this method, the probe combination comprises at least 4, but not more than 1000 probes. In some embodiments of this method, the probe combination comprises at least 4, but not more than 100 probes. In some embodiments of this method, the probe combination comprises at least 4, but not more than 10 probes.

In some embodiments of the method of determining the presence of metastatic oral squamous cell carcinoma, the method comprises amplification of target nucleic acids in chromosomal regions 3q, 8p, 8q, and 20. In some embodiments, this method comprises polymerase chain reaction (PCR) or multiplex ligation-dependent probe amplification (MLPA). In some embodiments, this method comprises producing a plurality of amplicons from a plurality of target nucleic acids in each chromosomal region. In some embodiments, this method comprises producing a plurality of amplicons from a plurality of target nucleic acids in each of one or more control chromosomal regions.

In some embodiments of the method of determining the presence of metastatic oral squamous cell carcinoma, the method comprises high-throughput DNA sequencing. In some embodiments, this method comprises sequencing a plurality of target nucleic acids in each chromosomal region. In some embodiments, this method comprises sequencing a plurality of target nucleic acids in each of one or more control chromosomal regions.

In another aspect, the invention provides a method of determining the presence of metastatic oral squamous cell carcinoma in a lymph node sample from a subject, the method comprising determining fraction of genome gained (FGG) and/or the fraction of genome altered (FGA) in the sample.

In some embodiments of the method of determining the presence of metastatic oral squamous cell carcinoma based on FGG or FGA, the method entails determining relative copy numbers for a plurality of target nucleic acids. In some embodiments of this method, the relative copy numbers are determined by analyzing genomic DNA. In some embodiments of this method, the relative copy numbers are determined by analyzing RNA, cDNA, or DNA amplified from RNA.

In some embodiments of the method of determining the presence of metastatic oral squamous cell carcinoma based on FGG or FGA, the method additionally comprises querying the copy number(s) of one or more control chromosomal regions.

In some embodiments of the method of determining the presence of metastatic oral squamous cell carcinoma based on FGG or FGA, the method is carried out by hybridization of sample nucleic acids to a combination of probes, which are immobilized on a substrate. In some embodiments, this method is carried out by array comparative genomic hybridization (aCGH). In some embodiments of this method, the combination of probes comprises a plurality of probes for each of one or more control chromosomal regions.

In some embodiments of the method of determining the presence of metastatic oral squamous cell carcinoma based on FGG or FGA, the method comprises amplification of target nucleic acids. In some embodiments, this method comprises polymerase chain reaction (PCR) or multiplex ligation-dependent probe amplification (MLPA). In some embodiments, this method comprises producing a plurality of amplicons from a plurality of target nucleic acids in each of one or more control chromosomal regions.

In some embodiments of the method of determining the presence of metastatic oral squamous cell carcinoma based on FGG or FGA, the method comprises high-throughput DNA sequencing. In some embodiments, this method comprises sequencing a plurality of target nucleic acids in each of one or more control chromosomal regions.

In some embodiments of the method of determining the presence of metastatic oral squamous cell carcinoma based on FGG or FGA, when the results of the method indicate the presence of metastatic oral squamous cell carcinoma, e.g., in a fine needle aspirate of a lymph node or a sentinel lymph node biopsy, the method additionally comprises removing one or more cervical lymph nodes from the subject. In cases of evaluating FGG and/or FGA in a lymph node, if the fraction of genome gained is above zero (0) and/or if the fraction of genome altered is above zero (0), metastatic oral squamous cell carcinoma is present in the sample.

Another aspect of the invention is a combination of probes or primers, wherein the probes or primers hybridize or anneal, respectively, to chromosomal regions 3q, 8p, 8q, and 20. The combination of probes or primers is capable of distinguishing samples including oral squamous cell carcinoma that is unlikely to metastasize, e.g., from samples that include oral squamous cell carcinoma that is likely to metastasize and/or that have a substantial likelihood of metastasis. In certain embodiments, the probes or primers hybridize or anneal, respectively, to chromosomal regions 3q24-qter, 8pter-p23.1, 8q12-q24.2, and 20pter-qter. In illustrative embodiments, chromosomal region 3q24-qter extends from SEQ ID NO:1 to the q terminus of chromosome 3, chromosomal region 8pter-p23.1 extends from the p terminus of chromosome 8 to SEQ ID NO:7, and chromosomal region 8q12-q24.2 extends from SEQ ID NO:11 to SEQ ID NO:4. In some embodiments, the combination includes one or more probes or primers that hybridize or anneal, respectively, to one or more control chromosomal regions. In certain embodiments, the combination of probes includes a plurality of probes for each chromosomal region. In variations of such embodiments, the combination of probes can include a plurality of probes for each of one or more control chromosomal regions. In some embodiments, the probe combination includes at least 4, but not more than about 10¹² probes, for example, not more than about 10¹¹ probes, 10¹⁰ probes, 10⁹ probes, 10⁸ probes, 10⁷ probes, 10⁶ probes, or 10⁵ probes. In illustrative embodiments, the combination includes at least 4, but not more than 10,000 probes or primers. In illustrative embodiments, the combination includes at least 4, but not more than 1000 probes or primers. In illustrative embodiments, the combination includes at least 4, but not more than 100 probes or primers. In some embodiments, the combination includes at least 4, but not more than 10 probes or primers.

The combination of probes or primers can be provided in a kit for distinguishing, identifying and/or diagnosing oral squamous cell carcinoma that is unlikely to metastasize. In some embodiments, the invention provides kits for distinguishing oral squamous cell carcinoma that is unlikely to metastasize from oral squamous cell carcinoma having a substantial likelihood of metastasis, comprising a combination of probes or primers that hybridize or anneal, respectively, to the chromosomal regions 3q, 8p, 8q, and 20. In various embodiments, the probes are immobilized on a substrate or the probes or primers labeled with different labels. In some embodiments, the kit further comprises one or more control probes or primers.

### BRIEF DESCRIPTION OF THE DRAWINGS

FIGS. 1A-F illustrate copy number aberrations involving 3q, 8p, 8q and chromosome 20 are frequent in oral dysplasia and occur at similar frequency in oral SCC. (A and B) Frequency of copy number aberrations shown in genome order in 29 oral dysplasia samples with no known association with cancer (A) and oral SCC cohort#1 (B). Gains are indicated by the red bars and losses by blue bars. Chromosome boundaries are indicated by vertical lines. (C and D) Hierarchical clustering based on genome-wide DNA copy number profile of 29 oral dysplasia samples with no known association with cancer (C) and oral SCC cohort#1 (D). Heatmaps were generated by unsupervised clustering of samples on trichotomous gain/loss/normal data for the autosomes. Euclidean distance d was used as the distance metric and Ward's linkage as the agglomeration method. Individual clones are represented as rows and ordered by chromosome and genome position according to the May 2004 freeze of the human genome (hg17). Clones on the p-arm are indicated either in light blue or yellow, and clones on the q-arm in dark blue or green. Acrocentric chromosomes are shown in green or dark blue. Columns represent individual tumor samples. Gains and losses were colored red and blue, respectively and focal amplifications yellow. Dysplasia grade is indicated (mild, light blue; moderate, dark blue; severe, purple), along with the TP53 mutation status of cases (TP53 mutant, dark blue; no detected mutation, light blue; TP53 status unknown, white). (E) Frequencies of gains of 3q, 8q, 20 and loss of 8p in oral dysplasia and SCC normalized to the total number of aberrations at these loci in each cohort. (F) Frequency of 3q8pq20 and non-3q8pq20 cases in oral dysplasia and SCC.

FIGS. 2A-B illustrate copy number aberrations involving 3q, 8p, 8q and chromosome 20 in oral dysplasia at sites of previous or subsequent cancers. (A) Frequency of aberrations plotted as in FIGS. 1A and B. (B) Hierarchical clustering based on genome-wide DNA copy number profiles of oral dysplasia samples associated with a previous and/or subsequent cancer as in FIG. 1C. Dysplasia grade and TP53 mutation status are indicated as in FIG. 1C. Cases with a previous cancer are indicated in light blue, a subsequent cancer in dark blue and both a previous and a subsequent cancer in pink.

FIGS. 3A-B illustrates copy number aberrations in oral SCC cohort#2. Frequency plot (A) and hierarchical clustering of cases showing nodal status (B) as in FIG. 1.

FIGS. 4A-B illustrate distribution of low level gains and losses among 3q8pq20 and non-3q8pq20 oral SCC cases. Hierarchical clustering based on genome-wide DNA copy number profiles of non-3q8pq20 (left) and 3q8pq20 (right) cases in SCC cohort#1 (A) and cohort#2 (B) as in FIGS. 1C and D. We assigned cases to the 3q8pq20 subtype if one or more of the aberrant regions at 3q, 8p, 8q and 20 as defined by >20% frequency in the dysplasia cohort with no association with cancer was present. The enhanced genomic instability associated with the 3q8pq20 subtype results in recurrent aberrations being more frequent in 3q8pq20 tumors (e.g., mean number of recurrent aberrations occurring at >15% frequency in cohort#1=4.53, range 1-13 compared to non-3q8pq20 tumors with mean=0.79, range 0-7).

FIGS. 5A-B illustrate distribution of low level gains and losses among 3q8pq20 and non-3q8pq20 oral SCC cases in cohorts #1 and #2. Comparison of frequencies of copy number gains (red) and losses (blue) for each clone in genome order in non-3q8pq20 and 3q8pq20 cases in SCC cohort#1 (A) and cohort#2 (B). Chromosome boundaries are indicated by solid vertical lines and positions of centromeres by dashed vertical lines. The bottom panel shows the level of significance of the difference (Fisher's exact test based on gain/loss/normal status) between the two sets of tumors at each clone. We excluded chromosome arms 3q, 8p, 8q and chromosome 20p and q, because regions from these chromosome arms were used to assign cases to each group. The significance levels shown by horizontal dashed lines are adjusted p-values, p=0.1 (green), 0.05 (blue) and 0.01 (red).

FIG. 6 illustrates association of 3q8pq20 and non-3q8pq20 subtypes with genome instability characteristics. Each boxplot represents the number of aberrations of different types involving autosomes. The thick horizontal line represents the median number of aberrations, while the bottom and top of each box represent the 25th and 75th percentile, respectively. The width of each box is proportional to the square root of the number of samples. Outlier values are indicated with circles. The p-values for each pairwise comparison are shown above the boxplots and were calculated using a two-sided Wilcoxon rank sum test. A p-value cut-off of 0.05 was used to declare significance. The number of cases in each group is shown below the group label.

FIG. 7 illustrates hierarchical clustering of samples and the 142 most variable methylation probes from (Poage et al. 2010) (NCBI GEO Accession GSE20939 and GSE20742). We show clustering of probes in rows, samples in columns and the 3q8pq20 status of the samples in the band across the top of the heatmap.

FIG. 8 illustrates enrichment of gene ontology (GO) processes represented by the significantly differentially methylated probes in highly unstable 3q8pq20 tumors from Poage et al. 2010 (NCBI GEO Accession GSE20939 and GSE20742). Shown are GO processes with more than four involved genes and p<0.02. The colored borders surrounding the gene names indicate increased (green) and decreased (blue) methylation. The thickness of the borders is proportional to the level of increased/decreased methylation.

FIG. 9 illustrates prediction of cervical nodal status by fraction of the genome gained (FGG) and altered (FGA). Shown are plots of FGG or FGA versus the cumulative number of node negative (N0) and node positive (N+) cases from SCC cohort#2. In this dataset, a clear cutpoint for prediction of nodal status is not evident by either measure. Nevertheless, by applying maximally selected Chi-square statistics (Rupert Miller & David Siegmund (1982). Maximally Selected Chi Square Statistics. Biometrics 38, 1011-1016), cutpoints at 0.065 and 0.095 were obtained for FGG and FGA, respectively, yielding sensitivity, specificity, positive predictive value and negative predictive value of 74%, 68%, 57% and 82% for FGG and 91%, 48%, 50% and 90% for FGA compared to 96%, 35%, 46% and 93% for 3q8pq20 status (Table 12). Thus, with these cutpoints, FGG and FGA both correctly identify more of the true N0 cases; however, more N+ cases are mistakenly called N0, which in the clinic may outweigh the benefits of detecting more N0 patients due to the extremely poor survival of patients who undergo surgical salvage for neck metastasis. Larger studies will be required to determine the utility of FGG, FGA and 3q8pq20 as biomarkers for cervical node status. For application in the clinic, however, it is likely that evaluation of 3q8pq20 (four loci) will have an advantage, since it would be more amenable to measurement using less complex biomarker assays (e.g., PCR) than would be assessment of genome-wide copy number alterations.

FIG. 10 illustrates survival with respect to nodal status of patients in cohort#2.

FIGS. 11A-M illustrate clone-wise association of clinical features with copy number alterations. Comparison of frequencies of copy number gains (red) and losses (blue) for each clone in genome order for N+ and N0 cases from cohort#2. Chromosome boundaries are indicated by solid vertical lines and positions of centromeres by dashed vertical lines. The bottom panel shows the level of significance of the difference (Fisher's exact test based on gain/loss/normal status) between the two sets of tumors at each clone. The significance levels shown by horizontal dashed lines are adjusted p-values.

FIG. 12 illustrates regions of amplification on 3q in oral SCC cohorts #1 and #2. We show copy number profiles for tumors from cohorts #1 and #2 for chromosome 3q, which define the boundaries of four regions of amplification. Candidate oncogenes (red) and tumor suppressor genes (blue) are indicated amongst the genes mapping to the four regions.

FIG. 13 illustrates two routes to cancer. Possible origin and progression of dysplastic lesions to cancers are differentiated by acquisition of +3q, −8p, +8q and/or +20 in dysplasia, which subsequently progress to 3q8pq20 oral SCC. Other lesions lacking these aberrations progress to non-3q8pq20 SCC. The 3q8pq20 and non-3q8pq20 cancers may arise from different cell types, a stem cell vs. a transit amplifying cell, for example.

FIG. 14 illustrates FISH analysis of oral mucosal brush biopsy. The oral site was brushed 10-15 times and the sample applied directly to a glass slide. Green probe=chr. 7 centromere, red=1q23.

FIGS. 15 A-C illustrate oral swabs. A-B. Isohelix swab (A) Swab. (B) integral tube and cap system (Photographs from Isohelix) C. Foam swab.

FIG. 16A-B illustrates array CGH with DNA from an oral SCC brushing. (a) Array CGH analysis of two independent brushings of a lesion. Shown are copy number ratios in genome order. Vertical lines indicate chromosome boundaries. A complex amplicon on 11q is evident in addition to detection of the same low level gains and losses in both samples. (b) Sequence trace showing detection of a TP53 mutation using DNA from the brushing. Methods: Each brush was deposited into a microfuge tube containing 500 μl of a tris-EDTA and SDS solution and DNA was isolated following overnight incubation with proteinase K, phenol chloroform extraction and ethanol precipitation.

### DETAILED DESCRIPTION

### In General

The present invention provides a molecular biomarker for the identification of tumors unlikely to metastasize. Tumor cells from an incisional biopsy or other source such as saliva or brushing of the tumor can be evaluated for the presence/absence of the molecular biomarker prior to surgical resection of the tumor, allowing the surgeon to determine whether the tumor is of the subtype that is unlikely to metastasize. This information can then be used in planning the surgical treatment, e.g., whether an elective neck dissection would be advised for a patient with a clinically N0 neck, i.e., where there is no evidence of regional lymph node involvement.

Oral epithelial dysplasia precedes and unpredictably transforms to oral squamous cell carcinoma (SCC). The present invention is based, in part, on the discovery that DNA copy number aberrations in chromosomal regions +3q24-qter, −8pter-p23.1, +8q12-q24.2 and +20 are early genomic events identifying two subgroups of dysplasia and cancers. One or more (e.g., two, three or four) of these aberrations is present in the major subgroup (termed 3q8pq20 subtype, comprising 70-80% of lesions) that develops with chromosomal instability, while they are absent from the more chromosomally stable non-3q8pq20 subgroup (20-30% of lesions). The 3q8pq20 subtype can be further subdivided according to level of genomic instability. The most chromosomally unstable 3q8pq20 tumors also display differential methylation compared to all other tumors and normal oral tissues. Little difference in methylation was detected when comparing the low instability 3q8pq20 and non-3q8pq20 tumors, suggesting that extensive epigenetic alterations do not contribute to formation of the non-3q8pq20 tumors. The 3q8pq20 and non-3q8pq20 cases, however, differ significantly in clinical outcome with risk for cervical (neck) lymph node metastasis almost exclusively associated with the 3q8pq20 subtype in two independent oral SCC cohorts. Thus, lack of +3q, −8p, +8q and +20 is a biomarker for low risk for oral SCC metastasis that can significantly alter clinical practice by identifying patients who do not require additional surgery to remove the cervical lymph nodes at the time of tumor resection. Moreover, while increased numbers of genomic alterations can be harbingers of progression to cancer, dysplastic lesions lacking copy number changes cannot be considered benign as they are potential precursors to non-3q8pq20 locally invasive, yet not metastatic oral SCC.

In particular, it has been discovered that oral SCC can be subdivided into those that harbor one or more (e.g., two, three or four) of the following: gains of regions on chromosome 3q and/or 8q, and/or loss of a region of 8p, and/or gain of chromosome 20; and those that do not have any of these aberrations. Tumors with one or more (e.g., two, three or four) of these aberrations are termed “3q8pq20,” and those lacking any of these aberrations, “non-3q8pq20.” The non-3q8pq20 group represents the minority of cases (20-30%). Non-3q8pq20 tumors are not associated with metastasis to the lymph nodes of the neck, compared with the 3q8pq20 tumors (p<0.006, Fisher test). This observation provides physicians with the capability to determine which patients require additional extensive surgery to remove the cervical (neck) lymph nodes at the time of the surgery to remove the tumor, and which patients could be spared this additional major surgery.

In addition to predicting substantial risk of metastasis of oral SCC, evaluation of relative copy number at chromosomal regions 3q8pq20 is useful for evaluating margins after tumor removal, for identifying dysplasias that, upon progression, are likely to progress to oral SCC that has a substantial risk of metastasis, for identifying dysplasias that could be monitored for possible progression, and for determining the presence of metastatic oral SCC (e.g., detecting micrometastases) in lymph nodes. With respect to evaluating tumor margins or dysplasias, a determination that a tumor or dysplasia is of the 3q8pq20 positive subtype (i.e., gains of regions on chromosome 3q and/or 8q, and/or loss of a region of 8p, and/or gain of chromosome 20), indicates that the tumor or dysplasia is more likely to have and/or acquire copy number alterations. Accordingly, monitoring margins and/or tumor recurrence and/or dysplasia progression by testing for copy number changes (e.g., by FISH) is useful for these cases.

With respect to evaluation of cancer cells in lymph nodes, there is currently interest in the use of sentinel lymph nodes to identify metastasis. Evaluation of copy number of 3q, 8p, 8q and/or 20 can aid the identification of tumor cells in the lymph nodes. Addition of molecular tests can increase sensitivity to detect micrometastases. Currently, immunohistochemistry for cytokeratins or RT-PCR for specific cancer-associated transcripts is used. Since FISH can be carried out on routinely fixed clinical specimens, there could be advantages over the use of RT-PCR, which requires that a portion of the node be frozen and not fixed. The studies described herein indicate that oral SCC metastases will have one or more (e.g., two, three or four) of the copy number changes, +3q, −8p, +8q and +20. Accordingly, tumor cells metastatic to the lymph node would also have one or more (e.g., two, three or four) of these aberrations. Small numbers of such cells can be identified in the lymph nodes, e.g., by FISH or any other appropriate method, with probes to these regions. Adding FISH to the analysis of the dissected lymph nodes improves the accuracy of the pathological assessment of nodal status.

### Method of Subtyping Oral SCC

In certain embodiments, the methods described herein are based, in part, on the identification of chromosomal regions that can be used to subtype oral SCC to determine whether an oral sample contains an SCC subtype that is substantially likely or unlikely to metastasize. The method entails obtaining an oral sample and analyzing it to determine nucleic acid copy number for regions of chromosomes 3q, 8p, 8q, and 20 relative to that for the rest of the genome (i.e., the “relative copy number”). For example, copy numbers for these regions can be compared to copy numbers for one or more other regions of the genome (e.g., one or more selected control regions) and/or compared to the average, median, or other representative copy number characteristic of the genome as a whole to determine copy number differences (i.e., gains or losses). In certain embodiments, copy numbers relative to one or more other regions and/or the average, median, or other representative copy number characteristic of the genome as a whole are determined for the following chromosomal regions: 3q24-qter, 8pter-p23.1, 8q12-q24.2, and 20pter-qter (i.e., the entire chromosome 20). Such comparisons can be carried out within a single cell, within pre-selected cells, or by bulk analysis.

Relative copy number can be determined by any available method, including in situ hybridization, array-based hybridization assays, amplification-based assays, and high-throughput DNA sequencing. In situ hybridization employs probes that reliably provide information on their targets in individual cells or chromosomes. Probes of these types are well known in the art and many are commercially available. The cells and chromosomes may be isolated from tissue or in the original tissue context.

Array-based hybridization and amplification-based assays typically employ nucleic acid extracted from the specimen and thus do not measure the copy number status of chromosomal regions of individual cells, unless only a single cell is subjected to the measurement. In such assays, a plurality of probes can be employed, and/or a plurality of target sequences amplified, across each of the chromosomal regions to obtain a sufficiently accurate representation of the relative copy number for the chromosomal region. When using high-throughput DNA sequencing for relative copy number determinations, it may also be desirable, in some embodiments, to sequence a plurality of sequences within each target chromosomal region. In various embodiments, the number of probes employed, and or target sequences amplified and/or sequenced, to ascertain the relative copy number of a particular chromosomal region is 1, 5, 10, 20, 30, 40, 50, 60, 70, 80, 90, 100, 200, 300, 400, 500, 600, 700, 800, 900, or 1000. Additionally, the number of probes employed, and or target sequences amplified and/or sequenced, can fall within any range bounded by any of these values.

In certain embodiments, it is advantageous to make copy number determinations at one or more control chromosomal regions, which are expected less frequently to have an altered copy number (relative to the average, median, or other representative copy number characteristic of the genome as a whole) in oral SCC. Control chromosomal regions include those that have been established by prior genomic studies of oral SCC to have a low frequency of copy number aberrations. In some embodiments, it may be desirable to make copy number determinations for a plurality of sequences within one or more control chromosomal regions. For example, multiple control region sequences can readily be queried in array-based hybridization and amplification assays, as well as determinations employing high-throughput DNA sequencing. In various embodiments, the number of control region sequences queried is 1, 5, 10, 20, 30, 40, 50, 60, 70, 80, 90, 100, 200, 300, 400, 500, 600, 700, 800, 900, 1000, 10⁴, 10⁵, 10⁶, 10⁷, 10⁸, 10⁹, 10¹⁰, 10¹¹, 10¹², or more, as appropriate, for each control region. Additionally, the number of sequences queried can fall within any range bounded by any of these values.

A relative copy number difference, gain or loss, is detected using any technique that is appropriate for the particular analytical method employed. Suitable techniques are well known and can be selected for a particular analytical method by one of skill in the art. Additional techniques may be developed in the future. In embodiments employing a labeled probe and/or primer, a gain can be detected as an elevated signal relative to the rest of the genome, e.g., relative to the signal from one or more control regions or relative to the average signal for the genome. Conversely, a loss can be detected as a reduced signal relative to the rest of the genome, e.g., relative to the signal from one or more control regions or relative to the average signal for the genome. The manner in which a signal from one or more labeled probe(s) and/or primer(s) is quantified will vary depending on the assay method. For example, for in situ hybridization, signal “level” can be determined by counting spots, whereas in other methods signal intensity is measured. The level to which this measured signal is compared can be predetermined or can be determined within the same assay by querying a control region, as discussed above, and/or by measuring signal level across the genome. Those of skill in the art appreciate that measuring signal level “across the genome” need not, and typically does not, entail querying every chromosomal locus, but rather querying a plurality of chromosomal loci, which can, e.g., be spaced across the genome. In some embodiments, the signal obtained from an oral SCC sample can be compared with that from a reference sample, which is typically obtained from non-cancerous tissue, to identify gains and losses in the oral SCC sample relative to the non-cancerous tissue.

Relative copy number can be determined by analyzing genomic DNA. In addition, indirect measurements of relative copy number can be obtained by analyzing RNA or nucleic acids derived from RNA, such as cDNA or DNA amplified from RNA. The relationship between relative copy number and expression levels of genes located in regions showing copy number differences is described, for example, in Pollack et al., Proc. Natl. Acad. Sci., USA 99:12963-68 (2002) (incorporated by reference here in its entirety and specifically for this description), which reports that, on average, a 2-fold change in DNA copy number is associated with a corresponding 1.5-fold change in mRNA levels. See also, Tonan et al. Proc. Natl. Acad. Sci., USA (102:9625-30 (2005) (incorporated by reference herein in its entirety and specifically for its description of RNA analysis in copy number determinations) and Carter et al., Nature Genetics 38:1043-48 (2006) (incorporated by reference herein in its entirety and specifically for its description of RNA analysis in copy number determinations). When analyzing mRNA (or DNA derived therefrom) to determine the relative copy number of a chromosomal region, in certain embodiments, the copy numbers (i.e., expression levels) of a plurality of transcripts, corresponding to a plurality of loci within the region are typically measured. In various embodiments, the number of different transcripts assessed for a particular region is up to about 3, 5, 10, 20, 30, 40, 50, 60, 70, 80, 90, 100, 200, 300, 400, 500, 600, 700, 800, 900, or 1000 or more. In certain embodiments, the copy number(s) (i.e., expression level(s)) of one or more control transcripts corresponding to genes whose expression level(s) is/are expected to be unaltered in oral SCC can be measured. For example, transcripts from one or more gene(s) in control chromosomal regions can be measured, e.g., in various embodiments, transcripts from up to about 3, 5, 10, 20, 30, 40, 50, 60, 70, 80, 90, 100, 200, 300, 400, 500, 600, 700, 800, 900, or 1000 or more genes in a control chromosomal region.

If the results indicate no gain of chromosomal regions 3q, 8q, and 20 and no loss of chromosomal region 8p, this finding indicates that the oral SCC is of a subtype that is unlikely to metastasize. In some embodiments, no gain of chromosomal regions 3q24-qter, 8q12-q24.2, and 20pter-qter and no loss of chromosomal region 8pter-p23.1 indicate an oral SCC that is unlikely to metastasize. The subject having this oral SCC can be treated for the oral SCC without removing the cervical lymph nodes.

If the results indicate a gain at one or more (e.g., two or three) of chromosomal regions 3q, 8q, and 20 and/or a loss of chromosomal region 8p, this finding indicates that the oral SCC is of a subtype that has a substantial likelihood of metastasizing. In some embodiments, a gain of one or more (e.g., two or three) of chromosomal regions 3q24-qter, 8q12-q24.2, and 20pter-qter and/or a loss of chromosomal region 8pter-p23.1 indicate an oral SCC for which there is a substantial likelihood that it has metastasized or that it will metastasize. In such a subject, the treatment for oral SCC can include removing the cervical lymph nodes.

Furthermore, the studies described herein show that the assessment of the fraction of the genome involved in DNA copy number gains (FGG) and the fraction that has any copy number alteration (FGA) are also strongly associated with risk of metastasis. Thus, 3q8pq20 status, FGG and/or FGA are all genomic biomarkers that can be useful in discriminating a subtype of oral SCC with a substantially high risk of metastasis from a subtype of oral SCC with a sufficiently low risk of metastasis to inform a significant aspect of clinical treatment (namely, the decision to remove cervical lymph nodes. Accordingly, in certain embodiments, the invention provides methods of determining the presence of oral squamous cell carcinoma that is substantially likely to metastasize, versus that which is unlikely to metastasize in an oral sample from a subject based on determining fraction of genome gained and/or the fraction of genome altered.

In particular embodiments, to measure the amount of the genome altered, each chromosomal region queried (e.g, each probe, such as a clone that is employed to probe a region) is assigned a genomic distance equal to the sum of one half the distance between its center and that of the neighboring chromosomal regions queried (e.g., neighboring clones). The genomic distances of clones that are gained or lost are summed and the resulting value represents the fraction of the genome altered (FGA). To calculate only the fraction of the genome gained or lost, only the genomic distances of clones that are gained or lost, respectively are considered. RNA expression levels can provide an indirect measure of the fraction of genome altered or gained or lost. See, e.g., Carter et al., Nature Genetics 38:1043-48 (2006) (incorporated by reference herein in its entirety and specifically for its description of RNA analysis in copy number determinations).

In various embodiments, a fraction of genome gained (FGG) below a threshold value of about 0.080, for example, below a threshold value of about 0.080, about 0.075, about 0.070, about 0.065, about 0.060, about 0.055, about 0.050, about 0.045, about 0.040, about 0.035, about 0.030, or about 0.025, indicates an oral SCC that is unlikely to metastasize, whereas an FGG above the threshold indicates an oral SCC having a substantial likelihood of metastasizing. In various embodiments, the threshold is about 0.065. In various embodiments, a fraction of genome altered (FGA) below a threshold of about 0.115, for example, below a threshold of about 0.110, about 0.105, about 0.100, about 0.095, about 0.090, about 0.085, about 0.080, about 0.075, about 0.070, about 0.065, about 0.060, about 0.055, about 0.050, about 0.045, about 0.040, about 0.035, about 0.030, or about 0.025, indicates an oral SCC that is unlikely to metastasize, whereas an FGA above the threshold indicates an oral SCC having a substantial likelihood of metastasizing. In various embodiments, the threshold is about 0.095. Additionally, the FGG or FGA threshold values can fall within any range bounded by any of the above-listed values for each (i.e., FGG or FGA) that are set forth above. By applying a lower threshold value, metastatic cases are more likely to be identified; however, this may lead to neck dissections on many patients who don't need it. Applying a higher threshold value spares patients unneeded neck surgery; however, patients with metastasis may not receive surgery (e.g., neck dissection to remove one or move cervical lymph nodes) and thus have a bad outcome. The applied threshold value depends on the judgment of a trained clinician, e.g., based on balancing the values of the various outcomes.

### Method of Subtyping Oral Epithelial Dysplasia

The invention further provides methods of assessing the risk, that if an oral epithelial dysplasia progresses, the oral epithelial dysplasia will progress to oral squamous cell carcinoma having a substantial likelihood of metastasis. These methods entail determining relative DNA copy numbers in a biological sample from a subject for the same chromosomal regions used for subtyping oral SCCs, namely 3q, 8p, 8q, and 20. A finding of no gain of chromosomal regions 3q, 8q, and 20, and no loss of chromosomal region 8p is indicative of oral epithelial dysplasia that, if it progresses, is unlikely to progress to metastatic oral squamous cell carcinoma. A finding of one or more of these copy number alterations, i.e., a gain of one or more (e.g., two or three) of chromosomal regions 3q, 8q, and 20, and/or a loss of chromosomal region 8p is indicative of oral epithelial dysplasia that, if it progresses, will progress to oral squamous cell carcinoma having a substantial likelihood of metastasis. The considerations for making these determinations (probes, methods, use of controls, etc.) are the same those described above for subtyping oral SCC, and specific aspects of such determinations are further described in the following sections.

If dysplasia of the 3q8pq20-positive subtype (i.e., gains of regions on chromosome 3q and/or 8q, and/or loss of a region of 8p, and/or gain of chromosome 20) progresses to cancer, it is likely to do so by the acquisition of further copy number alterations. Thus, one can monitor such dysplasias for progression using one or more probes to detect copy number alterations at chromosomal locations other than 3q, 8p, 8q and 20 that are frequently altered in oral SCC (see, e.g., Table 6). On the other hand, one would not expect non-3q8pq20 lesions to progress by the acquisition of further copy number alterations, so that evaluating these lesions for acquisition of copy number alterations would be unlikely to detect progression.

With respect to use of the non-3q8pq20 subtype for identifying patients at low risk of metastasis and use of the 3q8pq20-positive subtype for identifying patients having a substantially higher risk of metastasis, the 3q8pq20 biomarker can also be used together with current clinical assessments, e.g., tumor size, tumor thickness, tumor staging, to assist clinicians in providing a diagnosis and treatment regimen (e.g., whether to proceed with surgical treatment of the neck, i.e. neck dissection).

In embodiments where the DNA in the biological sample has a gain or loss of one or more (e.g., two, three or four) of said chromosomal regions (3q, 8p, 8q, and 20), i.e., is positive for the 3q8pq20 subtype, indicating an oral epithelial dysplasia that, if it progresses, will progress to oral squamous cell carcinoma having a substantial likelihood of metastasis, the methods can further comprise determining the dysplasia grade, and/or the presence of erythroplakia (a.k.a., erythroleukoplakia or leukoplakia). This can be done using any method known in the art, including without limitation visual inspection, palpation, and microscopic analysis. On visual examination, leukoplakia may vary from a barely evident, vague whiteness on a base of uninflamed, normal-appearing tissue to a definitive white, thickened, leathery, fissured, verrucous (wartlike) lesion. On palpation, some lesions may be soft, smooth, or finely granular. Other lesions may be roughened, nodular, or indurated. Malignant transformation to squamous cell carcinoma is seen in more than 15% of cases.

Histologic changes range from hyperkeratosis, dysplasia, and carcinoma in situ to invasive squamous cell carcinoma. The term “dysplasia” indicates abnormal epithelium and disordered growth, whereas the term “atypia” refers to abnormal nuclear features. Increasing degrees of dysplasia are designated as mild, moderate, and severe and are subjectively determined microscopically. Specific microscopic characteristics of dysplasia include (1) dropshaped epithelial ridges, (2) basal cell crowding, (3) irregular stratification, (4) increased and abnormal mitotic figures, (5) premature keratinization, (6) nuclear pleomorphism and hyperchromatism, and (7) an increased nuclear-cytoplasmic ratio.

It is generally accepted that the more severe the epithelial changes, the more likely a lesion is to evolve into cancer. When the entire thickness of epithelium is involved with these changes in a so-called top-to-bottom pattern, the term carcinoma in situ may be used. Designation of “carcinoma in situ” may also be used when cellular atypia is particularly severe, even though the changes may not be evident from basement membrane to surface. Carcinoma in situ is not regarded as a reversible lesion, although it may take many years for invasion to occur. A majority of squamous cell carcinomas of the upper aerodigestive tract, including the oral cavity, are preceded by epithelial dysplasia. Conceptually, invasive carcinoma begins when a microfocus of epithelial cell invades the lamina propria 1 to 2 mm beyond the basal lamina. At this early stage, the risk of regional metastasis is low. Further information on grading oral epithelial dysplasia can be found, e.g., in Regezi, et al., Oral Pathology: Clinical Pathologic Correlations, 5th edition (Oct. 2, 2007), Saunders.

Current management of dysplasia is based on the grade of dysplasia. Although there are a number of dysplasia grading systems that have been described, the most commonly used system is as follows. Mild dysplasias have architectural changes confined to the basal third of the full thickness of epithelium. Moderate dysplasias are up to two-thirds the full thickness of epithelium. Severe dysplasias are greater than two thirds of the full thickness, but without invasion through the basement membrane. Consideration is then given to the degree of cellular atypia. These features include increased nuclear cytoplasmic ratios, increased or abnormal mitoses, or pleomorphism of nuclei. Currently, the grading of dysplasia is used to predict risk. As many as 36% of severe dysplasias become invasive cancer (Silverman S, Jr., Gorsky M, Lozada F. Oral leukoplakia and malignant transformation. A follow-up study of 257 patients. Cancer. 1984 Feb. 1; 53(3):563-8; Schepman K P, van der Meij E H, Smeele L E, van der Waal I. Malignant transformation of oral leukoplakia: a follow-up study of a hospital-based population of 166 patients with oral leukoplakia from The Netherlands. Oral Oncol. 1998 July; 34(4):270-5; Lee J J, Hong W K, Hittelman W N, Mao L, Lotan R, Shin D M, et al. Predicting cancer development in oral leukoplakia: ten years of translational research. Clin Cancer Res. 2000 May; 6(5):1702-10). Cancer can also derive from hyperplasia or mild dysplasia, however. One group found that patients with mild dysplasia had the same transformation rates as those with severe dysplasia (Holmstrup P, Vedtofte P, Reibel J, Stoltze K. Long-term treatment outcome of oral premalignant lesions. Oral Oncol. 2006 May; 42(5):461-74).

In the context of the present invention, the assessment of the stage or monitoring of progression of an oral epithelial dysplasia positive for the 3q8pq20 subtype is helpful in assessing the need for, and timing of, aggressive interventions, such as excision of the dysplasia because, if such a dysplasia progresses, it will progress to oral squamous cell carcinoma having a substantial likelihood of metastasis. Any of the methods described herein or known in the art for assessing oral epithelial dysplasia can be carried out at the time of initial detection and at one or more time points thereafter separated by periods of 1, 2, 3, 4, 5, 6, 7, 8, 9, 10, or 11 months, or 1, 2, 3, 4, or 5 or more years, or any time period falling within a range bounded by any of the periods listed above.

Furthermore, in embodiments where the DNA in the biological sample has a gain or loss of one or more (e.g., two, three or four) of said chromosomal regions (3q, 8p, 8q, and 20), i.e., is positive for the 3q8pq20 subtype, indicating the oral dysplasia is likely to progress to metastatic oral squamous cell carcinoma, the methods described herein may further comprise more aggressively treating the oral dysplasia, e.g., including excising the dysplasia (e.g., by using a scalpel or laser excision) and chemoprevention.

The most common method for managing biopsy proven dysplasia of the oral cavity is local surgical excision. Excision of a dysplastic lesion provides a valuable histologic diagnosis. As mentioned above, 5% of idiopathic leukoplakias already have invasive cancer at the initial biopsy (Silverman S, Jr., Gorsky M, Lozada F. Oral leukoplakia and malignant transformation. A follow-up study of 257 patients. Cancer. 1984 Feb. 1; 53(3):563-8). In addition, incisional biopsies are subject to sampling error, and dysplasia or carcinoma can be easily missed. Some studies have reported that over 10% of lesions diagnosed by incisional biopsy as dysplasia demonstrated invasive carcinoma after excision (Chiesa F, Tradati N, Sala L, Costa L, Podrecca S, Boracchi P, et al. Follow-up of oral leukoplakia after carbon dioxide laser surgery. Arch Otolaryngol Head Neck Surg. 1990 February; 116(2):177-80; Thomson P J, Wylie J. Interventional laser surgery: an effective surgical and diagnostic tool in oral precancer management. Int J Oral Maxillofac Surg. 2002 April; 31(2):145-53). Evaluation of relative DNA copy number alterations at chromosomal regions (3q, 8p, 8q, and 20) to determine the 3q8pq20 subtype provides additional information to guide treatment and allow the provider and patient to make an informed decision regarding excision of a dysplastic lesion. A dysplasia that carries a higher risk of transforming into a metastatic oral cancer based on the method would have a stronger indication for surgical excision.

### Method of Determining the Presence of Metastatic Oral SCC in a Lymph Node Sample

The finding that copy number alterations at 3q, 8p, 8q, and 20 or oral SCC indicate likelihood of metastasis can also be exploited to identify oral SCC that has already metastasized by analyzing a lymph node sample for relative copy number alterations at these loci. In particular, a gain of one or more (e.g., two or three) of chromosomal regions 3q, 8q, and 20, and/or a loss of chromosomal region 8p is indicative of metastatic oral squamous cell carcinoma. In some embodiments, a gain of one or more (e.g., two or three) of chromosomal regions 3q24-qter, 8q12-q24.2, and 20pter-qter and/or a loss of chromosomal region 8pter-p23.1 is indicative of metastatic oral squamous cell carcinoma. In certain embodiments, one or more additional genetic alterations can be determined, such as fraction of genome gained (FGG), fraction of genome altered (FGA), methylation status, TP53 mutation(s), and the presence of relative copy number alterations at one or more loci other than 3q24-qter, 8pter-p23.1, 8q12-q24.2, and 20pter-qter.

Since the fraction of genome gained (FGG) and/or the fraction of genome altered (FGA) are also indicators of the likelihood of oral SCC metastasis, either or both of these parameters can be determined in a lymph node sample to identify the presence of metastatic oral squamous cell carcinoma in a lymph node sample. The considerations for determining 3q8pq20 status (probes, methods, use of controls, etc.) are the same as those described above for subtyping oral SCC, and specific aspects of such determinations are further described in the following sections). For this embodiment, an FGG and/or FGA value that is greater than zero (0) is an indication of cancer in the lymph node.

### Definitions

Terms used in the claims and specification are defined as set forth below unless otherwise specified.

The term “oral SCC” refers to a malignant neoplasm of oral tissue, such as, e.g., the tongue, gingiva, floor of mouth, retromolar trigone, buccal mucosa, and lip.

The terms “tumor” or “cancer” in an animal refer to the presence of cells possessing characteristics such as atypical growth or morphology, including uncontrolled proliferation, immortality, metastatic potential, rapid growth and proliferation rate, and certain characteristic morphological features. Often, cancer cells will be in the form of a tumor, but such cells may exist alone within an animal. The term tumor includes both benign and malignant neoplasms. The term “neoplastic” refers to both benign and malignant atypical growth.

The term “oral sample” is intended to mean a sample obtained from the oral cavity or surrounding tissue of a subject suspected of having, or having, oral SCC and/or dysplasia.

The terms “nucleic acid” or “polynucleotide,” as used herein, refer to a deoxyribonucleotide or ribonucleotide in either single- or double-stranded form. The term encompasses nucleic acids, i.e., oligonucleotides, containing known analogues of natural nucleotides which have similar or improved binding properties, for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6: 153-156).

The term “relative copy number” is used herein to refer to the nucleic acid copy number for a chromosomal region, relative to the copy number for another chromosomal region. In some cases, either one or both of the copy numbers may represent the average, median, mode etc. of one or more regions up to and including the whole genome. Relative copy number can be determined in any of a number of ways familiar to those of skill in the art. For example relative copy numbers can be determined by comparing a measured copy number value for a target chromosomal region to one or more measured copy number values for one or more other regions of the genome (e.g., one or more selected control regions) and/or to a copy number value for the rest of the genome, such as average, median, or other representative copy number characteristic of the genome as a whole.

The terms “copy number difference” and “altered copy number” refer to a difference in a copy number value for a chromosome region, e.g., a difference between a copy number value for a particular chromosomal region and a copy number value that is representative of the rest of the genome. In some cases either one or both of the copy numbers may represent the average, median, mode etc. of one or more regions up to and including the whole genome.

The terms “making a copy number determination” and “querying the copy number” refer to measuring any indication of nucleic acid copy number and do not require determining absolute copy number for any chromosomal region.

The term “substantial likelihood of metastasis” refers to the probability that an oral squamous cell carcinoma (SCC) has metastasized or will metastasize. In the context of the present invention, an oral SCC having no copy number alterations at any of the following chromosomal regions: 3q24-qter, 8pter-p23.1, 8q12-q24.2, and 20pter-qter is an oral SCC subtype at low risk for metastasis. As used herein, the term “substantial likelihood of metastasis” refers to a risk of metastasis, which is associated with an oral SCC that is not of this low-risk subtype.

The terms “hybridizing specifically to,” “specific hybridization,” and “selectively hybridize to,” as used herein, refer to the binding, duplexing, or hybridizing of a nucleic acid molecule preferentially to a particular nucleotide sequence under stringent conditions. The term “stringent conditions” refers to conditions under which a probe will hybridize preferentially to its target sequence, and to a lesser extent to, or not at all to, other sequences. A “stringent hybridization” and “stringent hybridization wash conditions” in the context of nucleic acid hybridization (e.g., as in array, Southern or Northern hybridization, or FISH) are sequence-dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions for filter hybridizations are selected to be about 5° C. lower than the thermal melting point (Tₘ) for the specific sequence at a defined ionic strength and pH, whereas for FISH the appropriate temperature difference may be 20 to 25° C. The Tₘ is the temperature (under defined ionic strength and pH) at which 50% of the target sequence hybridizes to a perfectly matched probe. Very stringent conditions are selected to be equal to the Tₘ for a particular probe. Dependency of hybridization stringency on buffer composition, temperature and probe length are well known to those of skill in the art (see, e.g., Sambrook and Russell (2001) Molecular Cloning: A Laboratory Manual (3rd ed.) Vol. 1-3, Cold Spring Harbor Laboratory, Cold Spring Harbor Press, NY, and detailed discussion, below).

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, more typically range from 10 to 30 nucleotides, or even more typically from 15 to 30 nucleotides, in length. Other primers can be somewhat longer, e.g., 30 to 50 nucleotides long. In this context, “primer length” refers to the portion of an oligonucleotide or nucleic acid that hybridizes to a complementary “target” sequence and primes nucleotide synthesis. Short primer molecules generally require cooler temperatures to form sufficiently stable hybrid complexes with the target. A primer need not reflect the exact sequence of the target but must be sufficiently complementary to hybridize with a target. A primer is said to anneal to another nucleic acid if the primer, or a portion thereof, hybridizes to a nucleotide sequence within the nucleic acid.

As used herein, with reference to a method performed by an individual, the term “amplification,” encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), digital amplification, and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18- (2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. No. 5,830,711, U.S. Pat. No. 6,027,889, U.S. Pat. No. 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

As those of skill in the art readily appreciate, the term “amplification” also refers to a chromosomal abnormality characterized by the gain of nucleic acid(s), and it will be clear to those of skill, from the context, whether this meaning is intended.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “label containing moiety” or “detection moiety” generally refers to a molecular group or groups associated with a probe, either directly or indirectly, that allows for detection of that probe upon hybridization to its target.

The term “target region” or “nucleic acid target” refers to a nucleotide sequence that resides at a specific chromosomal locus.

The term “control chromosomal region” refers to a chromosomal region that is not likely to have an altered copy number in oral SCC.

### Samples

Many types of oral samples from a patient having, or suspected of having, oral SCC can be employed in the methods described herein. Illustrative samples include saliva, an oral washing sample, an oral swab or brush sample, or an oral tissue sample, e.g., an incisional biopsy of the tumor from a site selected from the group consisting of: tongue, gingiva, floor of mouth, retromolar trigone, buccal mucosa, lip, or other oral site. In some embodiments, the sample is an incisional biopsy sample. The sample may be from the primary tumor, completely within a tumor or lesion (e.g., pre-cancerous or cancerous), or from the margin of a tumor or lesion. In various embodiments, a lymph node sample, e.g., a cervical lymph node sample, may be evaluated.

### Pre-Selection of Samples

Prior to detection, samples may be optionally pre-selected based on morphological characteristics, specific staining and the like. Pre-selection identifies suspicious cells, thereby allowing the relative copy number determination to be focused on those cells. Pre-selection increases the likelihood that the result will be correct. Pre-selection of a suspicious region on a tissue section may be performed on a serial section stained by conventional means, such as H&E or PAP staining, and the suspect region marked by a pathologist or otherwise trained technician. The same region can be located on the serial section stained by in situ hybridization and nuclei analyzed within that region, e.g, by in situ hybridization. Within the marked region, analysis may be limited to nuclei exhibiting abnormal characteristics as described above. Alternatively, the suspect region can be dissected from the tissue and analyzed by any applicable method including array-based hybridization assays, amplification-based assays, and high-throughput DNA sequencing. Single-cell analysis can be carried out, for example, using amplification-based assays

Similarly, in samples with dispersed cells such as saliva or brushings, cells with apparent cytologic abnormalities may be selected for analysis. During pre-selection involving dispersed cells, the cells can be placed on a microscope slide and visually scanned for cytologic abnormalities commonly associated with dysplastic and neoplastic cells. Such abnormalities include abnormalities in nuclear size, nuclear shape, and nuclear staining, as assessed by counterstaining nuclei with nucleic acid stains or dyes such as propidium iodide or 4,6-diamidino-2-phenylindole dihydrochloride (DAPI). Typically, neoplastic cells harbor nuclei that are enlarged, irregular in shape, and/or show a mottled staining pattern. Propidium iodide, typically used at a concentration of about 0.4 μg/ml to about 5 μg/ml, is a red-fluorescing DNA-specific dye that can be observed at an emission peak wavelength of 614 nm. DAPI, typically used at a concentration of about 125 ng/ml to about 1000 ng/ml, is a blue fluorescing DNA-specific stain that can be observed at an emission peak wavelength of 452 nm.

In certain embodiments, only those cells pre-selected for detection are subjected to analysis for chromosomal losses and/or gains. In some embodiments, pre-selected cells on the order of at least 20, at least 30, at least 40, at least 50, or at least 100, in number, are chosen for assessing chromosomal losses and/or gains. In other embodiments, cells to be analyzed may be chosen independent of cytologic or histologic features. For example, in in situ hybridization, all non-overlapping cells in a given area or areas on a microscope slide may be assessed for chromosomal losses and/or gains.

### Sample Processing

The sample can be processed or treated in any manner suitable for the analytical method to be employed. For example, samples to be analyzed by in situ hybridization can be treated with a fixative, such as formaldehyde, embedded in paraffin, and sectioned for use in the methods of the invention. Alternatively, fresh or frozen tissue can be pressed against glass slides to form monolayers of cells known as touch preparations, which contain intact nuclei and do not suffer from the truncation artifact of sectioning. These cells may be fixed, e.g., in alcoholic solutions such as 100% ethanol or 3:1 methanol:acetic acid. Nuclei can also be extracted from thick sections of paraffin-embedded specimens to reduce truncation artifacts and eliminate extraneous embedded material. Samples can also consist of cells obtained from saliva or brushings of oral lesions, which are then deposited on slides by well-known methods such as dropping, centrifugation or smearing. Typically, samples, once obtained, are harvested and processed prior to hybridization using standard methods known in the art. For in situ hybridization, such processing may include protease treatment and additional fixation in an aldehyde solution such as formaldehyde.

Sample nucleic acids can be extracted, using established methods, to the extent necessary to facilitate the analysis, e.g. high-throughput DNA sequencing. In some cases, the nucleic acid may be amplified prior to analysis. Sample nucleic acids are, in some embodiments, such as array CGH, labeled using any suitable labeling method. In some embodiments, genomic DNA is analyzed to determine relative copy number. In other embodiments, RNA, e.g, mRNA levels can be analyzed to determine relative copy number (i.e., expression analysis). In certain embodiments, RNA, or more specifically, mRNA, is converted to DNA, for example, by the use of reverse transcriptase to produce DNA or by amplification. If RNA is converted to DNA prior to the analysis, the method employed is preferably one that maintains the relative copy numbers of the transcripts. Such techniques are well known and suitable methods for particular applications can be selected by those of skill in the art.

### Probes

Some embodiments rely on the use of probes to detect relative copy number at particular loci.

In situ hybridization typically employs probes that can query the target chromosomal region of interest, i.e., can selectively bind to that region and provide a detectable signal. A probe to a particular chromosomal region can include multiple polynucleotide fragments, e.g., ranging in size from about 50 to about 1,000 nucleotides in length.

In situ hybridization probes that can be used in the method described herein include probes that selectively hybridize to chromosomal regions (e.g., 3q, 8p, 8q, and 20) or subregions of these chromosomal regions, i.e., 3q24-qter, 8pter-p23.1, 8q12-q24.2, and 20pter-qter (i.e., the entire chromosome 20). (The subregion designations as used herein include the designated band and typically about 10 megabases of genomic sequence to either side.) Probes useful in the in situ hybridization methods described herein include locus-specific probes and centromeric probes. A locus-specific probe selectively binds to a specific locus at a chromosomal region, e.g., 3q24-qter, 8pter-p23.1, 8q12-q24.2. A centromeric probe typically binds to repetitive sequences located at the centromere. Centromeric probes have been identified that selectively bind to the centromeric region of a particular chromosome and thus can be used to identify the presence of that region in a sample.

In situ hybridization probes that target a chromosomal region or subregion can readily be prepared by those of skill in the art or can be obtained commercially, e.g., from Abbott Molecular, Molecular Probes (Invitrogen, Life Technologies), or Cytocell (Oxfordshire, UK). Such probes are prepared using standard techniques, for example, from peptide nucleic acids, cloned human DNA such as plasmids, bacterial artificial chromosomes (BACs) (available from BACPAC, Oakland Calif.), and P1 artificial chromosomes (PACs) that contain inserts of human DNA sequences. Suitable probes may also be prepared, e.g., via amplification or synthetically.

Probes for assays other than in situ hybridization, for example quantitative PCR, are designed and employed to selectively hybridize to the target nucleic acids of interest. Probes can be perfectly complementary to the target nucleic acid sequence or can be less than perfectly complementary. In certain embodiments, probes anneal to the target sequence under stringent hybridization conditions.

Probes may also be employed as isolated nucleic acids immobilized on a solid surface (e.g., nitrocellulose, glass, silicon, beads), as in array Comparative Genomic Hybridization (aCGH). In some embodiments, the probes may be members of an array of nucleic acids as described, for instance, in WO 96/17958, which is hereby incorporated by reference in its entirety and specifically for its description of array CGH. Techniques capable of producing high density arrays are well-known (see, e.g., Fodor et al. Science 767-773 (1991) and U.S. Pat. No. 5,143,854), both of which are hereby incorporated by reference for this description. Customized arrays containing particular sequences are commercially available from such companies as Agilent, Nimblegen etc.

### Primers

Some embodiments employ primers to detect relative copy number at particular loci, e.g., amplification-based assays and high-throughput DNA sequencing. Primers suitable for nucleic acid amplification are sufficiently long to prime the synthesis of extension products in the presence of the agent for polymerization. The primers should be sufficiently complementary and sufficiently long to selectively anneal to their respective target sites and form stable duplexes. It will be understood that certain bases (e.g., the 3′ base of a primer) are generally desirably perfectly complementary to corresponding bases of the target nucleic acid sequence. In certain embodiments, primers anneal to the target sequence under stringent hybridization conditions.

One skilled in the art knows how to select appropriate primer pairs to amplify the target nucleic acid of interest. For example, PCR primers can be designed by using any commercially available software or open source software, such as Primer3 (see, e.g., Rozen and Skaletsky (2000) Meth. Mol. Biol., 132: 365-386; on the interne at broad.mit.edu/node/1060, and the like) or by accessing the Roche UPL website.

Primers may be prepared by any suitable method, including, for example, cloning and restriction of appropriate sequences or direct chemical synthesis or can be obtained from a commercial source.

### Hybridization/Annealing Conditions

Conditions for specifically hybridizing the probes and/or primers to their nucleic acid targets generally include the combinations of conditions that are employable in a given hybridization procedure to produce specific hybrids, which may easily be determined by one of skill in the art. Such conditions typically involve controlled temperature, liquid phase, and contact between a probe and a target. Hybridization conditions vary depending upon many factors including probe/primer concentration, target length, target and probe/primer G-C content, solvent composition, temperature, and duration of incubation. At least one denaturation step may precede contact of the probes/primers with the targets. Alternatively, both the probe/primer and nucleic acid target may be subjected to denaturing conditions together while in contact with one another, or with subsequent contact of the probe/primer with the biological sample. Hybridization may be achieved with subsequent incubation of the probe/primer/sample in, for example, a liquid phase that is compatible with subsequent steps of the assay. For example if no subsequent enzymatic amplification is required the liquid phase may comprise about a 50:50 volume ratio mixture of 2-4×SSC and formamide, at a temperature in the range of about 25 to about 55° C. Higher hybridization temperatures are typically employed if formamide is not included in the liquid. Temperatures are also adjusted based on the length of the complementary sequences that are participating in the hybridization. Hybridization times range from about several seconds for PCR primers to about 96 hours. In order to increase specificity, use of a blocking agent such as unlabeled blocking nucleic acid as described in U.S. Pat. No. 5,756,696 (the contents of which are herein incorporated by reference in their entirety, and specifically for the description of the use of blocking nucleic acid), may be employed in conjunction with the methods of the present invention. Other conditions may be readily employed for specifically hybridizing the probes/primers to their nucleic acid targets present in the sample, as would be readily apparent to one of skill in the art.

Upon completion of a suitable incubation period, non-specific binding of probes to sample DNA may be removed by one or a series of washes. Temperature, salt, and formamide etc. concentrations are suitably chosen for a desired stringency. The level of stringency required depends on the complexity of a specific probe sequence in relation to the genomic sequence, and may be determined by systematically hybridizing probes to samples of known genetic composition. In general, high stringency washes without formamide may be carried out for conventional nucleic acids at a temperature in the range of about 65 to about 80° C. with about 0.2× to about 4×SSC and about 0.1% to about 1% of a non-ionic detergent such as Nonidet P-40 (NP40). If lower stringency washes are required, the washes may be carried out at a lower temperature with an increased concentration of salt.

### Detection

Hybridization

The hybridization of probes can be detected using any means known in the art. Label-containing moieties can be associated directly or indirectly with probes. Different label-containing moieties can be selected for each individual probe within a particular combination so that each hybridized probe is visually distinct from the others upon detection. Where FISH or NanoString® methodologies are employed, the probes can be conveniently labeled with distinct fluorescent label-containing moieties. In such embodiments, fluorophores, organic molecules that fluoresce upon irradiation at a particular wavelength, are typically directly attached to the probes. A large number of fluorophores are commercially available in reactive forms suitable for DNA labeling.

Attachment of fluorophores to nucleic acid probes is well known in the art and may be accomplished by any available means. Fluorophores can be covalently attached to a particular nucleotide, for example, and the labeled nucleotide incorporated into the probe using standard techniques such as nick translation, random priming, PCR labeling, and the like. Alternatively, the fluorophore can be covalently attached via a linker to the deoxycytidine nucleotides of the probe that have been transaminated. Methods for labeling probes are described in U.S. Pat. No. 5,491,224 and Molecular Cytogenetics: Protocols and Applications (2002), Y.-S. Fan, Ed., Chapter 2, “Labeling Fluorescence In situ Hybridization Probes for Genomic Targets,” L. Morrison et al., p. 21-40, Humana Press, both of which are herein incorporated by reference for their descriptions of labeling probes.

Exemplary fluorophores that can be used for labeling probes include TEXAS RED (Molecular Probes, Inc., Eugene, Oreg.), CASCADE blue aectylazide (Molecular Probes, Inc., Eugene, Oreg.), SPECTRUMORANGE™ (Abbott Molecular, Des Plaines, Ill.) and SPECTRUMGOLD™ (Abbott Molecular).

One of skill in the art will recognize that other agents or dyes can be used in lieu of fluorophores as label-containing moieties. Luminescent agents include, for example, radioluminescent, chemiluminescent, bioluminescent, and phosphorescent label-containing moieties. Silver or gold, as well as isotopic mass tags, can also be employed as labeling agents. Detection moieties that are visualized by indirect means can be used. For example, probes can be labeled with biotin or digoxygenin using routine methods known in the art, and then further processed for detection. Visualization of a biotin-containing probe can be achieved via subsequent binding of avidin conjugated to a detectable marker. The detectable marker may be a fluorophore, in which case visualization and discrimination of probes may be achieved as described above for FISH.

Probes hybridized to target regions may alternatively be visualized by enzymatic reactions of label moieties with suitable substrates for the production of insoluble color products. Each probe may be discriminated from other probes within the set by choice of a distinct label moiety. A biotin-containing probe within a set may be detected via subsequent incubation with avidin conjugated to alkaline phosphatase (AP) or horseradish peroxidase (HRP) and a suitable substrate. 5-bromo-4-chloro-3-indolylphosphate and nitro blue tetrazolium (NBT) serve as substrates for alkaline phosphatase, while diaminobenzidine serves as a substrate for HRP.

In embodiments where fluorophore-labeled probes or probe compositions are used, the detection method can involve fluorescence microscopy, flow cytometry, or other means for determining probe hybridization. Any suitable microscopic imaging method may be used in conjunction with the methods of the present invention for observing multiple fluorophores. In the case where fluorescence microscopy is employed, hybridized samples may be viewed under light suitable for excitation of each fluorophore and with the use of an appropriate filter or filters. Automated digital imaging systems such as the MetaSystems, BioView or Applied Imaging systems may alternatively be used. Alternatively, the assay format may employ the methodologies described in Direct Multiplexed Measurement of Gene Expression with Color-Coded Probe Pairs (Geiss, et al., Nat Biotechnol. (2008) 26(3):317-25), which describes the nCounter™ Analysis System (nanoString Technologies). This system captures and counts individual hybridized nucleic acids by a molecular bar-coding technology, and is commercialized by Nanostring (on the internet at nanostring.com). See also, WO 2007/076128; and WO 2007/076129.

In Situ Hybridization

The hybridization signals for the set of probes to the target regions is detected and recorded for cells chosen for assessment of chromosomal losses and/or gains. Hybridization is detected by the presence or absence of the particular signals generated by each of the probes. Hybridization may also be performed to a reference sample with known gains and losses to assist with the analysis, for example a sample of normal cells that do not have any gains or losses. Once the copy number of target regions within each cell is determined, as assessed by the number of hybridization signals for each probe, relative chromosomal gains and/or losses may be quantified. The quantification of losses/gains can include determinations that evaluate the ratio of copy number of one locus to another on the same or a different chromosome.

Several methods can be used to determine whether a sample contains one or more of the copy number aberrations identified by the present invention. When a control sample of normal cells is employed, the relative gain or loss for each probe is determined by comparing the number of distinct probe signals in each cell to the number expected in a normal cell, i.e., where the relative copy number should be two. Non-neoplastic cells in the sample, such as keratinocytes, fibroblasts, and lymphocytes, can be used as reference normal cells. More than the normal number of probe signals is considered a gain, and fewer than the normal number is considered a loss. Alternatively, a minimum number of signals per probe per cell can be required to consider the cell abnormal (e.g., 5 or more signals). Likewise for loss, a maximum number of signals per probe can be required to consider the cell abnormal (e.g., 0 signals, or one or fewer signals). Still alternatively, a sample may have all loci elevated in copy number compared to normal cells (e.g. a tetraploid tumor) and in such cases it is of interest which loci may be more highly or less highly elevated.

The percentages of cells with at least one gain and/or loss are to be recorded for each locus. A cell is considered abnormal if at least one of the genetic aberrations identified by a probe combination of the present invention is found in that cell. A sample may be considered positive for a gain or loss if the percentage of cells with the respective gain or loss exceeds the cutoff value for any probes used in an assay. Alternatively, two or more loci with apparent aberrant copy number can be required in order to consider the cell abnormal at the desired region, with the effect of increasing specificity. Still alternatively, the total number of signals from all selected cells in the sample at each measured locus may be compared to the other measured loci in order to determine if at least one of the aberrations identified by a probe combination of the present invention is present in the sample.

aCGH

In array CGH, the probes are not labeled, but rather are immobilized at distinct locations on a substrate, as described in WO 96/17958. In this context, the probes are often referred to as the “target nucleic acids.” The sample nucleic acids are typically labeled to allow detection of hybridization complexes. The sample nucleic acids used in the hybridization may be detectably labeled prior to the hybridization reaction. Alternatively, a detectable label may be selected which binds to the hybridization product. In dual- or multi-color aCGH, the target nucleic acid array is hybridized to two or more collections of differently labeled nucleic acids, either simultaneously or serially. For example, sample nucleic acids (e.g., from oral SCC biopsy) and reference nucleic acids (e.g., from normal oral tissue) are each labeled with a separate and distinguishable label. Differences in intensity of each signal at each target nucleic acid spot can be detected as an indication of a copy number difference. Although any suitable detectable label can be employed for aCGH, fluorescent labels are typically the most convenient.

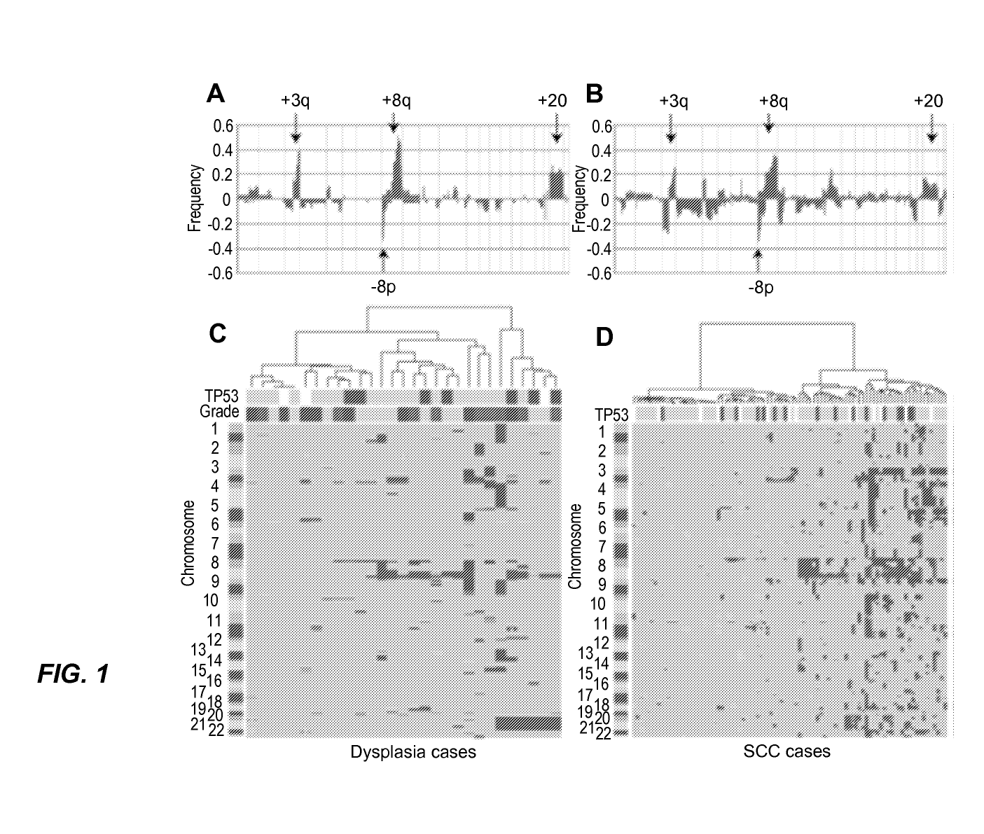

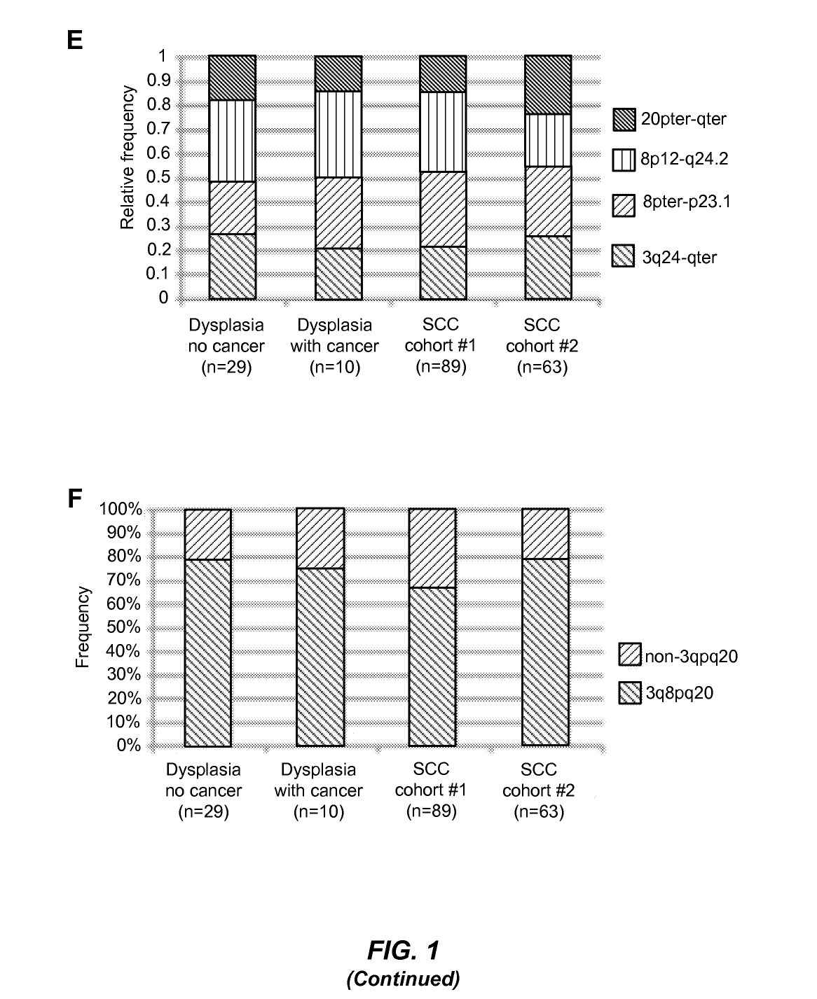

Array CGH can be carried out in single-color or dual- or multi-color mode. In single-color mode, only the sample nucleic acids are labeled and hybridized to the nucleic acid array. Copy number differences can be detected by detecting signal intensities for all of the probes on the array, normalizing those intensities by comparing them to intensities from control samples known to have normal DNA copy number at essentially all loci, and then comparing the normalized intensities for the sample nucleic acid to determine if there are loci that are at increased or decreased copy number relative to the average for the genome. To facilitate this determination, the array can include target elements for one or more loci (“control loci”) that are not expected to show copy number difference(s) in oral SCC. Control loci can be selected based on the data in FIG. 1.

In dual- or multi-color mode, signal corresponding to each labeled collection of nucleic acids (e.g., sample nucleic acids and normal, reference nucleic acids) is detected at each target nucleic acid spot on the array. The signals at each spot can be compared, e.g., by calculating a ratio of the sample to the normal reference signal at each locus, and normalizing the signals so that the average, median, modal ratio for the entire genome is 1.0. Then, if the normalized ratio of sample nucleic acid signal to reference nucleic acid signal at a target spot significantly exceeds 1, this indicates a gain in the sample nucleic acids at the locus corresponding to the target nucleic acid spot on the array. Conversely, if the ratio of sample nucleic acid signal to reference nucleic acid signal is significantly less than 1, this indicates a loss in the sample nucleic acids at the corresponding locus.

Array-based relative copy number determinations can be obtained using a commercial service, such as, e.g., the Affymetrix-authorized SeqWright.

Amplification-Based Detection

In still another embodiment, amplification-based assays can be used to measure the relative copy numbers at loci within chromosomal regions. In such amplification-based assays, the target nucleic acids act as template(s) in amplification reaction(s) (e.g., Polymerase Chain Reaction (PCR)). In a quantitative amplification, the amount of amplification product is proportional to the amount of template in the original sample. Detailed protocols for quantitative PCR are provided in Innis et al. (1990) PCR Protocols, A Guide to Methods and Applications, Academic Press, Inc. N.Y.). A number of commercial quantitative PCR systems are available, for example the TaqMan system from Applied Biosystems.

Other suitable amplification methods include, but are not limited to, ligase chain reaction (LCR) (see Wu and Wallace (1989) Genomics 4: 560; Landegren et al. (1988) Science 241: 1077; and Barringer et al. (1990) Gene 89: 117), multiplex ligation-dependent probe amplification (MLPA), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Amplification is typically carried out using primers that specifically amplify one or more loci within each chromosome (e.g., chromosome 20), chromosomal region (e.g., 3q, 8p, and 8q), or chromosomal subregion (e.g., 3q24-qter, 8pter-p23.1, and 8q12-q24.2) to be queried. Detection can be carried out by any standard means, including a target-specific probe, a universal probe that binds, e.g., to a sequence introduced into all amplicons via one or both primers, or a double-stranded DNA-binding dye (such as, e.g., SYBR Green). In illustrative embodiments, padlock probes or molecular inversion probes are employed for detection.

Padlock probes (PLPs) are long (e.g., about 100 bases) linear oligonucleotides. The sequences at the 3′ and 5′ ends of the probe are complementary to adjacent sequences in the target nucleic acid. In the central, noncomplementary region of the PLP there is a “tag” sequence that can be used to identify the specific PLP. The tag sequence is flanked by universal priming sites, which allow PCR amplification of the tag. Upon hybridization to the target, the two ends of the PLP oligonucleotide are brought into close proximity and can be joined by enzymatic ligation. The resulting product is a circular probe molecule catenated to the target DNA strand. Any unligated probes (i.e., probes that did not hybridize to a target) are removed by the action of an exonuclease. Hybridization and ligation of a PLP requires that both end segments recognize the target sequence. In this manner, PLPs provide extremely specific target recognition.

The tag regions of circularized PLPs can then be amplified and resulting amplicons detected. For example, TaqMan® real-time PCR can be carried out to detect and quantify the amplicon. The presence and amount of amplicon can be correlated with the presence and quantity of target sequence in the sample. For descriptions of PLPs see, e.g., Landegren et al., 2003, Padlock and proximity probes for in situ and array-based analyses: tools for the post-genomic era, Comparative and Functional Genomics 4:525-30; Nilsson et al., 2006, Analyzing genes using closing and replicating circles Trends Biotechnol. 24:83-8; Nilsson et al., 1994, Padlock probes: circularizing oligonucleotides for localized DNA detection, Science 265:2085-8.

Molecular inversion probes (MIPs) are often employed in single nucleotide polymorphism (SNP) analysis. Like padlock probes, MIPs are single-stranded DNA molecules containing two regions complementary to regions in the target nucleic acid that flank a SNP in question. Each probe also contains universal primers' sequences separated by an endodeoxyribonuclease recognition site and a 20-nt tag sequence. During the assay the probes undergo a unimolecular rearrangement: they are (1) circularized by filling gaps with nucleotides corresponding to the SNPs in four separate allele-specific polymerization (A, C, G, and T) and ligation reactions; (2) linearized in an enzymatic reaction. As a result they become “inverted.” This step is followed by amplification. The use of MIPs is described further in Absalan F, Ronaghi M., “Molecular inversion probe assay.” Methods Mol Biol. 2007; 396:315-30; and Hardenbol P et al., “Multiplexed genotyping with sequence-tagged molecular inversion probes.” Nat Biotechnol. 2003 June; 21(6):673-8. Epub 2003 May 5.

High-Throughput DNA Sequencing

In particular embodiments, amplification methods are employed to produce amplicons suitable for high-throughput (i.e., automated) DNA sequencing. Generally, amplification methods that provide substantially uniform amplification of target nucleotide sequences are employed in preparing DNA sequencing libraries having good coverage. In the context of automated DNA sequencing, the term “coverage” refers to the number of times the sequence is measured upon sequencing. The counts obtained are typically normalized relative to a reference sample or samples to determine relative copy number. Thus, upon performing automated sequencing of a plurality of target amplicons, the normalized number of times the sequence is measured reflects the number of target amplicons including that sequence, which, in turn, reflects the number of copies of the target sequence in the sample DNA.

Amplification for sequencing may involve emulsion PCR isolates in which individual DNA molecules along with primer-coated beads are present in aqueous droplets within an oil phase. Polymerase chain reaction (PCR) then coats each bead with clonal copies of the DNA molecule followed by immobilization for later sequencing. Emulsion PCR is used in the methods by Marguilis et al. (commercialized by 454 Life Sciences), Shendure and Porreca et al. (also known as “Polony sequencing”) and SOLiD sequencing, (developed by Agencourt, now Applied Biosystems). Another method for in vitro clonal amplification for sequencing is bridge PCR, where fragments are amplified upon primers attached to a solid surface, as used in the Illumina Genome Analyzer. Some sequencing methods do not require amplification, for example the single-molecule method developed by the Quake laboratory (later commercialized by Helicos). This method uses bright fluorophores and laser excitation to detect pyrosequencing events from individual DNA molecules fixed to a surface. Pacific Biosciences has also developed a single molecule sequencing approach that does not require amplification.

After in vitro clonal amplification (if necessary), DNA molecules that are physically bound to a surface are sequenced. Sequencing by synthesis, like dye-termination electrophoretic sequencing, uses a DNA polymerase to determine the base sequence. Reversible terminator methods (used by Illumina and Helicos) use reversible versions of dye-terminators, adding one nucleotide at a time, and detect fluorescence at each position in real time, by repeated removal of the blocking group to allow polymerization of another nucleotide. Pyrosequencing (used by 454) also uses DNA polymerization, adding one nucleotide species at a time and detecting and quantifying the number of nucleotides added to a given location through the light emitted by the release of attached pyrophosphates.

Pacific Biosciences Single Molecule Real Time (SMRT™) sequencing relies on the processivity of DNA polymerase to sequence single molecules and uses phospholinked nucleotides, each type labeled with a different colored fluorophore. As the nucleotides are incorporated into a complementary DNA strand, each is held by the DNA polymerase within a detection volume for a greater length of time than it takes a nucleotide to diffuse in and out of that detection volume. The DNA polymerase then cleaves the bond that previously held the fluorophore in place and the dye diffuses out of the detection volume so that fluorescence signal returns to background. The process repeats as polymerization proceeds.

Sequencing by ligation uses a DNA ligase to determine the target sequence. Used in the Polony method and in the SOLiD technology, this method employs a pool of all possible oligonucleotides of a fixed length, labeled according to the sequenced position. Oligonucleotides are annealed and ligated; the preferential ligation by DNA ligase for matching sequences results in a signal informative of the nucleotide at that position.

In various embodiments, affinity capture or other enrichment procedures can be used to enrich sequences from particular parts of the genome for subsequent sequencing. Such enrichment methods are known in the art.

### Probe Combinations and Kits for Use in Oral SCC Subtyping and Related Methods

The invention includes combinations of probes and/or primers, as described herein, that can be used to subtype oral SCC or oral epithelial dysplasia or to detect metastatic oral SCC in a lymph node, as well as kits for use in diagnostic, research, and prognostic applications. Kits include probe/primer combinations and can also include reagents such as buffers and the like. The kits may include instructional materials containing directions (i.e., protocols) for the practice of the methods of this invention. While the instructional materials typically include written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. The kit may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims.

In addition, all other publications, patents, and patent applications cited herein are hereby incorporated by reference in their entirety for all purposes.

### EXAMPLES

### Example 1

### Oral Squamous Cell Carcinoma Copy Number Aberrations Associated with a Subtype that is Unlikely to Metastasize

Clinically evident precancerous oral lesions preceding development of oral squamous cell carcinomas (SCC) include oral epithelial dysplasia of varying grades (mild, moderate, severe) (5). Transformation to cancer occurs in 16% of mild and 55% of moderate/severe dysplasia and is considered to occur by stepwise acquisition of genetic and/or epigenetic alterations (6). The data in this study show that +3q24-qter, −8pter-p23.1, +8q12-q24.2 and +20 occur at ≧20% frequency in oral dysplasia cases with no known association with oral cancer. Moreover, 75-80% of all dysplasia and SCC cases harbor one or more of these copy number aberrations, with additional recurrent aberrant regions occurring in SCC. On the other hand, 20-25% of dysplasia and oral SCC cases lack the copy number aberrations +3q, −8p, +8q and +20, and have few or no other copy number alterations. Thus, aberrations involving 3q, 8p, 8q and chromosome 20 appear to be early events that identify a major subgroup of oral cancer (3q8pq20 subtype) that develops with chromosomal instability, and distinguishes it from a smaller group of chromosomally stable SCC (non-3q8pq20). Importantly, the two subtypes differ in clinical behavior, with the non-3q8pq20 tumors being associated with a low risk for metastasis. Presence of one or more of the aberrations, +3q, −8p, +8q and +20, is therefore a biomarker for oral SCC metastasis. In addition, while increased numbers of genomic alterations can be harbingers of progression to cancer, lesions lacking copy number changes cannot be considered benign as they are potential precursors to the 20-25% of oral SCC that lack recurrent copy number alterations.

It is generally accepted that oral SCC develops via accumulation of genetic and epigenetic changes in a multi-step process, with aberrations being frequently recognized in premalignant lesions or in histologically normal tissue (6). On the one hand, several independent reports support loss of heterozygosity (LOH) at 9p21 and 3p as early events in development of dysplasia, with LOH at additional loci associated with transformation to cancer (7). On the other hand, studies purporting to show that aneuploidy alone was the best predictor of progression to cancer were subsequently discovered to have been founded on fabricated data (8). Therefore, to clarify the role of genomic aberrations in oral cancer progression and metastasis, array comparative genomic hybridization (CGH) was carried out to determine the genome-wide spectrum of copy number gains and losses in 39 oral dysplasia samples, 29 with no known association with cancer and 10 that either subsequently progressed to cancer or appeared at the site of a previous cancer.

### Methods

Patients and Tissue Samples.

We obtained formalin fixed paraffin embedded dysplasia and SCC tissue specimens from oral cavity sites (tongue, gingiva, floor of mouth, retromolar trigone, buccal mucosa and lip) and associated clinical data through the UCSF Oral Cancer Tissue Bank and Cancer Registry. Patient consent was obtained for use of all specimens. For cohort#2, we considered oral cavity SCC cases treated at the University of California San Francisco Medical Center between 1998-2005 to be eligible for inclusion if patients were older than 21 years and they did not receive radiation or chemotherapy prior to tumor resection. We considered cases to be node positive if the histopathologic nodal status was positive at the time of surgical treatment or metastasis was identified during the five year follow up period, whereas we considered patients to be node negative if pathologic nodal status was negative at the time of surgical resection and no nodal involvement occurred during a five year follow up period. From the 2500 cases in the bank, we were able to identify and accession tissue blocks for 64 cases for which the required clinical information was available and there was sufficient tumor material (i.e. tumors≧1.5 cm in diameter) for analysis. Prior to extraction of nucleic acids from dysplasia or SCC specimens, we stained the first and last sections with hematoxylin and eosin. We examined these sections to confirm the diagnosis and grading of dysplasia, which was done by one pathologist (RCKJ), and to estimate the normal cell content of the regions of dysplasia and SCC selected for dissection, which varied from 60-90% epithelial cells. Patient samples and characteristics are provided in Tables 1 and 2.

TP53 Sequencing.

We amplified exons 5-8 of TP53 from genomic DNA and carried out cycle sequencing, as described previously (Snijders et al. 2005).

Array CGH.

We dissected regions of dysplasia or tumor from 15 consecutive 10 μm formalin fixed paraffin embedded tissue sections from routine surgical excisions. For the analysis of cohort#2, we also dissected regions of normal tissue, e.g. muscle from the same patient blocks. We extracted DNA and carried out copy number measurements on arrays of 2464 BAC clones printed in triplicate as described previously (Snijders et al. 2005). The array datasets are available at NCBI GEO (submission in progress).

Array Data Pre-Processing.

We studied four datasets. We obtained two datasets from previous publications; SCC cohort#1 from our own published work (Snijders et al. 2005) and an independent dataset from the Netherlands (Smeets et al. 2009). Here, we describe the analysis of the two new datasets. The oral dysplasia dataset comprises 39 samples hybridized to three different print versions of the UCSF BAC array (Snijders et al. 2001) (HumArray2.0, 3.0, and 3.2), which differ slightly in clone content. The oral SCC dataset (cohort#2) comprises 63 tumor samples, with accompanying paired normal samples from the same patient for 61 of cases. All of the tumor samples were hybridized to the HumArray3.2 platform. We used UCSF SPOT (Jain et al. 2002) for array image analysis, and after quality filtering on spots and targets, we applied a “SpotCorrection” algorithm for removing systematic geometric and GC content effects. The algorithm employs an iterative scheme to estimate smoothly varying spatial artifacts in log 2 ratios across the array while retaining ‘true’ (genomically coherent) signals. We normalized GC content using loess and then performed replicate spot averaging and clone filtering as previously described (Snijders et al. 2003). We estimated the experimental variability of each CGH profile (sd) by taking the median of the absolute deviations (MAD) of the measurements on clones with the same copy number in that profile, and if replicate hybridizations were available for a case, we retained the one with the lower MAD.

For a subset of the oral dysplasia samples hybridized on the HumArray 2.0 platform, we observed a “print batch effect” (PBE), which manifested as systematic enhanced noise across multiple samples. To correct this effect, we clustered the data from these samples, which revealed two different PBE populations. For each of these, we calculated a PBE template as the median log 2 ratio per probe (BAC clone) across samples in each population. After appropriate scaling (equal to the dot product amplitude of the tumor profile on the template), we subtracted the PBE template from the tumor profile.

For tumor profiles with paired normal hybridization profiles, we applied noise reduction using the normal sample as template. The success of this strategy implies the existence of a shared sample specific effect on the log 2 ratios across hybridizations. The magnitude of this effect can be estimated using the derivative log ratio spread (DLRS) (Chen et al. 2008). The scaling factor between a tumor profile and its normal template was the ratio of their respective DLRS's. For the two tumor profiles without a paired normal, we employed the per-probe median of the normal profiles as a normal template, with DLRS scaling as above.

Statistical Methods.

All p-values less than 0.05 were considered significant, unless there was a multiple comparisons adjustment, in which case a q-value less than 0.05 was considered significant. Calculations were performed using the R language (Ihaka and Gentleman 1996).

Copy Number Analysis.

We mapped the dysplasia and SCC data to the May 4 freeze of the human genome sequence (hg17) and separately processed each dataset using circular binary segmentation (CBS) (Olshen et al. 2004) as implemented in the DNAcopy package that is part of Bioconductor (Gentleman et al. 2004). We used the scaled median absolute deviation (MAD) of the difference between the observed and segmented values to estimate the sample-specific experimental variation. For each sample, we declared a segment to be gained or lost if the average log 2 ratio was at least two times the sample MAD away from the median segmented value. We defined high level amplifications, as we have described previously (Fridlyand et al. 2006b), by considering the width of the segment to which a clone belonged and the minimum difference between the segment value of the clone and the segment means of the neighboring segments. We declared a clone amplified if it belonged to the segment spanning less than 20 Mb and the minimum difference was greater than exp(−x3) where x is the difference in segment means.

We calculated the numbers and types of genomic alterations as described previously (Fridlyand et al. 2006b). Briefly, we defined the total number of copy number transitions (break points) as the total number of segments minus the number of chromosomes. The number of whole arm changes (centromeric copy number transitions), we defined as occurring when the segment end was assigned at the most proximal clone on the p-arm. We assigned whole chromosome changes to chromosomes without identified breakpoints and when the chromosomal segment mapped to the gain or loss level. Finally, we scored an autosomal chromosome arm as amplified if it contained at least one amplified clone.

To measure the amount of the genome altered, we assigned each clone a genomic distance equal to the sum of one half the distance between its center and that of its neighbouring clones. We summed the genomic distances of clones that are gained or lost and the resulting value represents the fraction of the genome altered (FGA). To calculate only the fraction of the genome gained or lost, we considered only the genomic distances of clones that are gained or lost, respectively.

Hierarchical Clustering of Tumor Profiles.

We grouped our samples and generated heatmaps by unsupervised clustering of samples on trichotomous gain/loss/normal data for the autosomes. We used Euclidean distance as the distance metric and Ward's linkage as the agglomeration method.

Determination of Recurrent Regions of Aberration.

We defined recurrent common regions of aberration as contiguous clones for which the frequency of gain (or loss) occurred at greater than or equal to a specified frequency in a cohort. Within each recurrent region, we also defined recurrent focal regions as any local maxima in the frequency. In a new sample, we considered a previously specified region to be “gained” if more clones were gained than lost, “lost” if more clones were lost than gained, and “normal” if there were no gains or losses. Counts of aberrant regions were compared using the Wilcoxon rank sum test.

To identify samples as 3q8pq20, we defined recurrent common regions using a frequency of >20% in the dysplasia cohort with no known association with cancer. We declared samples to be 3q8pq20 if one or more of the common recurrent gains on 3q, 8q or 20 (encompassing a focal region on 20p including JAG1) or loss of 8p was present. Proportions of 3q8pq20 subjects were compared between cohorts using Fisher's exact test.

Evaluation of Significant Differences in Recurrent Aberrations in Dysplasia and SCC.

We compared dysplasias and SCC cohort#1 for differences in aberrations of chromosome arms or recurrent regions of aberration. For the region-wise comparison, we used a frequency cutoff of 20% in SCC cohort#2. Differences were evaluated using Fisher's exact test (Mehta 1986) utilizing the dichotomized indicator gained (or lost)/not gained (or not lost), and the p-values were adjusted for multiple testing by controlling the false discovery rate (FDR) (Benjamini 1995).

Evaluation of Differences Between 3q8pq20 and Non-3q8pq20 Tumors in SCC Cohorts #1 and #2.

Similar to the above analysis for regional differences, we identified differences in aberration frequencies in individual clones between 3q8pq20 and non-3q8pq20 cases in SCC cohort#1 and #2 utilizing Fisher's exact test. Differences in instability characteristics in SCC cohort#2 were evaluated using the Wilcoxon rank sum test.

Copy Number and Methylation Analysis.

Copy number and methylation data for a head and neck cancer data set comprised of 15 oral cavity and 4 oropharyngeal tumors (Poage et al. 2010) were accessioned from NCBI GEO (GSE20939 and GSE20742). Segmentation of the copy number data (Olshen et al. 2004) revealed low amplitude copy number changes, suggestive of normal cell contamination, requiring assignment of 3q8pq20 status to the oral cavity cases by visual inspection of the copy number profiles. We further distinguished whether 3q8pq20 cases had high or lower levels of copy number alterations.

Methylation data consisted of beta values on 1413 probes for 26 samples (15 tumors and 11 controls). The following nonlinear transformation was applied to the beta values,

s=sqrt(beta)−sqrt(1−beta).

This transformation increases the Gaussian character of the data and has the effect of reducing the number of false positives. The transformed data were then quantile normalized across samples. We used the top 10% most variable probes (142 probes, Table 9) for hierarchical clustering, which was performed using Euclidean distance and complete linkage. Probes were tested for differential methylation between tumor types using the limma package, for the following comparisons: highly unstable 3q8pq20 vs. the rest; all tumors vs. the normal cases; and 3q8pq20 tumors vs. non-3q8pq20 tumors plus normal cases. The probes for each comparison were filtered on absolute mean difference in methylation level (>0.05) and adjusted p-value (<0.05, FDR) (Benjamini 1995). This analysis yielded 49, 18 and 15 probes for the above three comparisons, respectively (Table 10). To generate the list of probes differentially methylated only in the highly unstable 3q8pq20 tumors, we removed probes from the highly unstable 3q8pq20 vs. the rest list if they were included in any of the other comparisons leaving 37 probes (Table 10).

We used EGAN (Paquette and Tokuyasu 2010) to investigate enrichments in the probes differentially methylated in the highly unstable 3q8pq20 tumors. For the analysis, we generated a background gene list from the GPL9183 annotations file for the Illumina array (NCBI GEO, GSE20939 and GSE20742), and we used the probe with the minimum p-value, if a gene were represented by multiple probes (32 genes).

Associations with Clinical Characteristics.

We compared patient and tumor characteristics with 3q8pq20 status, cervical node status and genome instability measures using Fisher's exact test. We estimated survival curves by nodal status using the Kaplan-Meier method, and we tested for differential survival using the log-rank test.

### Results

### Copy Number Aberrations Distinguish Two Oral Dysplasia and Cancer Subtypes

We assembled a cohort of 39 oral dysplasia cases comprised of lesional biopsies from 29 cases with no known association with cancer and 10 from patients who subsequently developed cancer at the site of the dysplasia or the dysplasia appeared at the site of a previous cancer (Table 1 and Table 2). We compared these profiles to those of oral SCCs from two independent cohorts, cohort#1 (89 cases), which we had previously profiled (Snijders, et al., Oncogene (2005); 24: 4232-42) and cohort#2 with 63 cases with five-year clinical follow-up (Table 1 and Table 3).

TABLE 1
Summary of clinical characteristics of dysplasia and oral SCC cohorts.

| | Dysplasia | Dysplasia | | |
|---|---|---|---|---|
| | (no known association | (associated | SCC | SCC |
| | with cancer) | with cancer) | cohort #1 | cohort #2 |
| Age | | | | |
| <65 | 20 (69%) | 6 (60%) | 47 (53%) | 30 (48%) |
| ≧65 | 9 (31%) | 4 (40%) | 42 (47%) | 33 (52%) |
| Sex | | | | |
| female | 9 (31%) | 4 (40%) | 47 (53%) | 26 (41%) |
| male | 20 (69%) | 6 (60%) | 42 (47%) | 37 (59%) |
| Grade | | | | |
| mild | 12 (41%) | 3 (30%) | NA | NA |
| moderate | 8 (28%) | 4 (40%) | NA | NA |
| severe | 9 (31%) | 3 (30%) | NA | NA |
| moderately differentiated | NA | NA | 35 (39%) | 42 (67%) |
| moderate to poorly differentiated | NA | NA | 4 (4%) | 3 (5%) |
| moderate to well differentiated | NA | NA | 6 (7%) | 1 (2%) |
| poorly differentiated | NA | NA | 5 (6%) | 4 (6%) |
| well differentiated | NA | NA | 39 (44%) | 13 (21%) |
| Site | | | | |
| buccal mucosa | 4 (14%) | 0 | 17 (19%) | 9 (14%) |
| floor of mouth | 2 (7%) | 0 | 17 (19%) | 11 (17%) |
| gingiva | 1 (3% ) | 1 (10%) | 21 (24%) | 11 (17%) |
| palate | 1 (3%) | 0 | 0 | 2 (3%) |
| tongue | 21 (72%) | 7 (70%) | 34 (38%) | 21 (33%) |
| retromolar trigone | 0 | 1 (10%) | 0 | 5 (8%) |
| lower lip | 0 | 1 (10%) | 0 | 0 |
| floor of mouth, tongue | 0 | 0 | 0 | 2 (3%) |
| floor of mouth, tongue, buccal mucosa | 0 | 0 | 0 | 1 (2%) |
| floor of mouth, tongue, gingiva | 0 | 0 | 0 | 1 (2%) |
| TP53 mutation status | | | | |
| wild type | 19 (66%) | 3 (30%) | 59 (66%) | NA |
| mutant | 7 (24%) | 2 (20%) | 16 (18%) | NA |
| unknown | 3 (10%) | 5 (50%) | 14 (16%) | NA |
| Cancer association | | | | |
| previous | unknown | 4 (40%) | NA | NA |
| subsequent | unknown | 5 (50%) | NA | NA |
| previous and subsequent | unknown | 1 (10%) | NA | NA |
| Tumor size (cm) | | | | |
| <2.7 | NA | NA | NA | 29 (46%) |
| ≧2.7 | NA | NA | NA | 33 (52%) |
| unknown | NA | NA | NA | 1 (2%) |
| Tumor thickness (cm) | | | | |
| <1.3 | NA | NA | NA | 24 (38%) |
| ≧1.3 | NA | NA | NA | 26 (41%) |
| unknown | NA | NA | NA | 13 (21%) |
| Clinical node status | | | | |
| negative | NA | NA | NA | 25 (40%) |
| positive | NA | NA | NA | 14 (22%) |
| unknown | NA | NA | NA | 24 (38%) |
| Pathological node status | | | | |
| N0 | NA | NA | NA | 40 (63%) |
| N+ | NA | NA | NA | 23 (37%) |
| Recurrence | | | | |
| not-recurred | NA | NA | NA | 49 (78%) |
| recurred | NA | NA | NA | 12 (19%) |
| unknown | NA | NA | NA | 2 (3%) |
| Vital status | | | | |
| survived/censored | NA | NA | NA | 25 (40%) |
| dead | NA | NA | NA | 38 (60%) |
| Tumor status | | | | |
| free | NA | NA | NA | 44 (70%) |
| not free | NA | NA | NA | 15 (24%) |
| unknown | NA | NA | NA | 4 (6%) |
| Alcohol use | | | | |
| current | NA | NA | NA | 25 (40%) |
| never used | NA | NA | NA | 11 (17%) |
| previous use | NA | NA | NA | 7 (11%) |
| unknown | NA | NA | NA | 20 (32%) |
| Tobacco use | | | | |
| current cigarette smoker | NA | NA | NA | 19 (30%) |
| never used | NA | NA | NA | 12 (19%) |
| previous use | NA | NA | NA | 14 (22%) |
| current snuff/smokeless tobacco user | NA | NA | NA | 1 (2%) |
| unknown | NA | NA | NA | 17 (27%) |

NA = not applicable

TABLE 2
Dysplasia cases

| | | | | | | TP53 | Prior | Cancer | |
|---|---|---|---|---|---|---|---|---|---|
| | Patient | | | | | Sequence | Cancer | progression | |
| OCRC# | ID | Grade | Site | Sex | Age | exons 5-8 | (months) | (months) | Amplification |
| 5707 | 3297 | mild | tongue | M | 49 | NA | Unknown | Unknown | |
| 5724 | 2860 | mild | tongue | F | 53 | no mutation | Unknown | Unknown | |
| 5749 | 3329 | severe | tongue | M | 64 | no mutation | Unknown | Unknown | |
| 5769 | 3346 | mild | tongue | M | 82 | exon 5, H179R | Unknown | Unknown | |
| | | | | | | (CAT > CGT) | | | |
| 5779 | 3354 | moderate | tongue | F | 23 | NA | Unknown | Unknown | 2q11.2, |
| | | | | | | | | | 21q21.3 |
| 5807 | 3377 | mild | tongue | M | 52 | exon 6, H193L | Unknown | Unknown | |
| | | | | | | (CAT > CTT) | | | |
| 5824 | 2215 | severe | tongue | M | 62 | no mutation | Unknown | Unknown | |
| | | | | | | (exons 5, 7, 8) | | | |
| 5905 | 3436 | moderate | tongue | M | 42 | NA | Unknown | Unknown | |
| 5914 | 1921 | moderate | tongue | M | 56 | no mutation | Unknown | Unknown | 2q11.2 |
| 5952 | 3470 | moderate | FOM | M | 77 | exon 6, I195S | Unknown | Unknown | CCND1, |
| | | | | | | (ATC > AGT) | | | PAK1 |
| 6162 | 3539 | mild | tongue | F | 66 | no mutation | Unknown | Unknown | |
| 6201 | 3665 | moderate | gingiva | F | 71 | exon 8, P278T | Unknown | Unknown | |
| | | | | | | (CCT > ACT) | | | |
| 6390 | 287 | moderate | tongue | F | 60 | no mutation | Unknown | Unknown | JAG1 |
| 6402 | 3784 | mild | tongue | M | 57 | no mutation | Unknown | Unknown | |
| 6419 | 2734 | severe | tongue | M | 70 | no mutation | Unknown | Unknown | |
| 6427 | 3801 | mild | tongue | F | 73 | exon 6, T211I | Unknown | Unknown | |
| | | | | | | (ACT > ATT) | | | |
| 6463 | 3832 | severe | tongue | M | 50 | no mutation | Unknown | Unknown | |
| 6475 | 3839 | severe | palate | F | 52 | no mutation | Unknown | Unknown | |
| 6486 | 2578 | mild | FOM | M | 50 | no mutation | Unknown | Unknown | |
| 6686 | 3981 | mild | buccal | M | 61 | no mutation | Unknown | Unknown | |
| | | | mucosa | | | | | | |
| 6689 | 3983 | severe | buccal | M | 81 | no mutation | Unknown | Unknown | |
| | | | mucosa | | | | | | |
| 6690 | 3984 | mild | tongue | M | 60 | no mutation | Unknown | Unknown | |
| 6695 | 3989 | mild | buccal | M | 41 | exon 5, S127F | Unknown | Unknown | |
| | | | mucosa | | | (TCC > TTC) | | | |
| 6756 | 4036 | mild | tongue | F | 50 | no mutation | Unknown | Unknown | |
| 7453 | 3467 | moderate | tongue | M | 56 | no mutation | Unknown | Unknown | |
| 7618 | 4865 | severe | tongue | M | 82 | exon 5, H179R | Unknown | Unknown | |
| | | | | | | (CAT > AAT) | | | |
| 7646 | 4622 | severe | tongue | M | 57 | no mutation | Unknown | Unknown | |
| | | | | | | (exons 5, 7, 8) | | | |
| 7678 | 4649 | severe | buccal | F | 74 | no mutation | Unknown | Unknown | |
| | | | mucosa | | | | | | |
| 7694 | 4662 | moderate | tongue | M | 42 | no mutation | Unknown | Unknown | |

Associated
with cancer

| | | | | | | | | | |
|---|---|---|---|---|---|---|---|---|---|
| 5653 | 3223 | severe | tongue | F | 81 | NA | Yes, −1 | Unknown | |
| 5809 | 3332 | mild | lower | M | 64 | no mutation | Yes, −2 | Unknown | |
| | | | lip | | | | | | |
| 8292 | 5097 | severe | tongue | M | 71 | NA | Yes, −2 | Unknown | |
| 8444 | 769 | mild | tongue | M | 51 | NA | Yes, −348 | Unknown | |
| 6889 | 4127 | severe | tongue | M | 62 | het. deleletion | Yes, ~140 | Yes, +33 | 7q21.12, |
| | | | | | | exon 5 | | | 7q21.12, |
| | | | | | | (TTTGCCAAC | | | 9p13.3, |
| | | | | | | TGGCCAA) | | | 11q22, 13q, |
| | | | | | | | | | 21q |
| 149 | 74 | moderate | tongue | F | 83 | NA | Unknown | Yes, +41 | |
| 5681 | 3271 | moderate | gingiva | F | 86 | exon 8, | Unknown | Yes, +21 | |
| | | | | | | R282W (CGG > | | | |
| | | | | | | TGG) | | | |
| 5922 | 3450 | mild | tongue | M | 47 | no mutation | Unknown | Yes, +49 | |
| 6367 | 2071 | moderate | retromolar | M | 56 | no mutation | Unknown | Yes, +3 | |
| | | | trigone | | | | | | |
| 8417 | 5234 | moderate | tongue | F | 49 | NA | Unknown | Yes, +20 | 2q11.2, |
| | | | | | | | | | CCND1, |
| | | | | | | | | | PAK1 |

TABLE 3A
Characteristics of patients in cohort#2

| | Final | | | | Tumor | Tumor | Node | |
|---|---|---|---|---|---|---|---|---|
| | Node | Age at | | | size | thickness | status | |
| ID# | status | Diagnosis | Sex | Site | (cm) | (cm) | (clinical) | Histology* |
| AB003 | N0 | 70 | M | Retromolar Region | 1 | 1 | 0 | MD |
| AB004 | N0 | 68 | M | Gingiva | 5 | 1.7 | X | MD |
| AB007 | N+ | 48 | M | Floor of Mouth | 10 | 5.5 | 2c | WD |
| AB010 | N0 | 61 | M | Tongue | 1.5 | 1.5 | X | MD |
| AB011 | N0 | 47 | M | Tongue | 2 | 1.4 | 0 | MD |
| AB014 | N+ | 59 | M | Retromolar Region | 3.2 | 1.7 | 2 | MD |
| AB015 | N+ | 68 | F | Tongue | 5.2 | 3.9 | X | MD |
| AB016 | N+ | 74 | M | Buccal Mucosa | 2.3 | 0.9 | X | PD |
| AB017 | N+ | 59 | M | Buccal Mucosa | 5 | NR | 0 | WD |
| AB018 | N+ | 50 | M | Floor of Mouth | 3.6 | 0.6 | 1 | MD |
| AB019 | N+ | 60 | F | Floor of Mouth, | 4.5 | 3.5 | 2c | MD |
| | | | | tongue | | | | |
| AB020 | N+ | 86 | F | Hard Palate | 0.9 | NR | 1 | WD |
| AB021 | N0 | 68 | F | Tongue | 3.2 | NR | X | MD |
| AB022 | N0 | 60 | M | Floor of Mouth | 0.9 | 0.6 | X | MD |
| AB023 | N0 | 41 | M | Tongue | 3.2 | 2.2 | 0 | WD |
| AB025 | N0 | 69 | F | Gingiva | 2.4 | 0.2 | 0 | MD |
| AB026 | N0 | 65 | F | Retromolar Region | 2 | 0.8 | 0 | MD to PD |
| AB027 | N0 | 80 | M | tongue | 4.4 | NR | 0 | MD to PD |
| AB029 | N0 | 64 | F | Floor of Mouth, | 3 | 0.8 | 2 | PD |
| | | | | tongue, buccal | | | | |
| | | | | mucosa | | | | |
| AB030 | N0 | 58 | M | Floor of Mouth | 0.9 | NR | X | MD |
| AB031 | N0 | 78 | F | Tongue | 5.2 | NR | 0 | WD |
| AB032 | N0 | 46 | M | Buccal Mucosa | 8 | NR | NR | MD |
| AB033 | N+ | 77 | F | Retromolar Region | 6.3 | 2.2 | 1 | MD |
| AB034 | N0 | 69 | M | Buccal Mucosa | 6.4 | NR | 0 | WD |
| AB035 | N0 | 83 | M | Tongue | 4.7 | 1.3 | 1 | MD |
| AB037 | N+ | 64 | M | Floor of Mouth | 2.7 | NR | NR | MD |
| AB038 | N+ | 66 | M | Tongue | 6 | 3.2 | 2C | MD |
| AB039 | N0 | 75 | M | Gingiva | 3 | 1.5 | 0 | MD |
| AB040 | N+ | 49 | F | Tongue | 9.2 | 4.6 | X | MD |
| AB041 | N0 | 51 | M | Tongue | NR | NR | 0 | MD |
| AB042 | N0 | 65 | M | Tongue | 3 | 1.5 | 0 | MD |
| AB045 | N0 | 76 | M | Gingiva | 2.7 | 1 | 2c | WD |
| AB047 | N0 | 46 | F | Buccal Mucosa | 1 | 0.5 | 0 | MD |
| AB048 | N0 | 46 | M | Tongue | 1.5 | 0.3 | X | MD |
| AB049 | N0 | 46 | M | Tongue | 1.1 | 0.7 | X | MD |
| AB051 | N+ | 43 | M | Tongue | 3 | 0.9 | 0 | MD |
| AB052 | N+ | 51 | M | Tongue | 6 | 3.5 | X | MD to WD |
| AB054 | N+ | 76 | F | Buccal Mucosa | 4.2 | 2.9 | X | PD |
| AB055 | N0->N+ | 57 | M | Floor of Mouth | 1.6 | 1.6 | X | MD |
| AB056 | N+ | 83 | M | Retromolar Region | 4 | 3.6 | 2b | MD |
| AB059 | N+ | 68 | F | Tongue | 1.2 | 0.35 | 0 | MD |
| AB060 | N+ | 57 | F | Tongue, Floor of | 1.7 | 1.5 | 0 | MD |
| | | | | Mouth | | | | |
| AB061 | N0 | 39 | M | Buccal Mucosa | 1.3 | 0.5 | 0 | WD |
| AB062 | N0 | 56 | M | Gingiva | 1 | 0.4 | NR | MD |
| AB063 | N0 | 49 | M | Tongue | 1 | 0.8 | 0 | MD |
| AB064 | N0 | 39 | M | Buccal Mucosa | 1.5 | 0.5 | 0 | MD |
| AB065 | N0 | 85 | F | Gingiva | 2.5 | 1 | 0 | MD |
| AB066 | N0 | 57 | M | Tongue | 1.7 | 1.4 | 0 | MD |
| AB067 | N0 | 81 | F | Floor of Mouth | 8 | NR | NR | MD to PD |
| AB068 | N0 | 70 | F | Gingiva | 2 | 0.5 | X | WD |
| AB069 | N0 | 56 | M | Buccal Mucosa | 1 | 0.6 | NR | WD |
| AB070 | N0 | 71 | M | Floor of Mouth | 2.5 | 0.3 | NR | WD |
| AB071 | N0 | 90 | F | Hard Palate | 3.5 | 3 | 0 | MD |
| AB073 | N+ | 96 | F | Gingiva | 2.7 | 1.7 | x | |
| AB076 | N0 | 81 | F | Gingiva | 2.5 | NR | | MD |
| AB077 | N0 | 61 | F | Floor of Mouth | 2.3 | NR | 0 | MD |
| AB079 | N0 | 77 | F | Tongue | 4.8 | 1.4 | 2B | WD |
| AB080 | N0 | 81 | F | Tongue | 3.3 | 2.4 | 0 | PD |
| AB081 | N+ | 83 | F | Gingiva | 4.4 | 1.8 | X | MD |
| AB082 | N+ | 70 | M | Floor of Mouth | 2.9 | 1.3 | 1 | MD |
| AB083 | N+ | 54 | F | Floor of Mouth, | 7 | 5 | X | MD |
| | | | | tongue, gingiva | | | | |
| AB084 | N+ | 57 | F | Gingiva | 1.1 | 0.5 | 1 | MD |
| AB085 | N0 | 79 | F | Tongue | 0.8 | 0.8 | X | MD |
| AB086 | N0 | 77 | M | Floor of Mouth | 1.8 | 1.2 | 0 | WD |

*WD: well differentiated, PD: poorly differentiated, MD: moderately differentiated;
NR: not reported

TABLE 3B
Characteristics of patients in cohort#2

| | | Total | | | | | |
|---|---|---|---|---|---|---|---|
| | Recurrence, | Follow-up | Vital | Tumor | Node status | | |
| ID# | Type | (Months) | Status | Status | (path) | Alcohol | Tobacco |
| AB003 | Local | 20 | DEAD | Unknown | 0 | Unknown | Previous |
| AB004 | None | 85 | DEAD | Free | 0 | Past | Current |
| AB007 | Regional | 19 | DEAD | Not free | 2C | None | None |
| AB010 | None | 126 | ALIVE | Free | 0 | None | None |
| AB011 | None | 86 | ALIVE | Free | 0 | Current | Previous |
| AB014 | None | 34 | ALIVE | Free | 1 | None | None |
| AB015 | Distant mets | 22 | DEAD | Not free | 2C | None | None |
| AB016 | None | 18 | DEAD | Unknown | 2B | Past | Current |
| AB017 | None | 5 | DEAD | Free | 2B | Current | Current |
| AB018 | None | 60 | ALIVE | Free | 2C | Current | Previous |
| AB019 | None | 8 | DEAD | Free | 2C | Current | Current |
| AB020 | None | 4 | DEAD | Not free | N1 | None | None |
| AB021 | Local | 36 | DEAD | Not free | 0 | Unknown | Unknown |
| AB022 | None | 100 | ALIVE | Free | 0 | Current | Current |
| AB023 | Local | 80 | ALIVE | Free | 0 | Current | Current |
| AB025 | None | 44 | ALIVE | Free | 0 | Current | Unknown |
| AB026 | None | 15 | DEAD | Free | 0 | Current | Current |
| AB027 | None | 2 | DEAD | Not free | 0 | None | None |
| AB029 | Local | 105 | ALIVE | Free | 0 | Current | Current |
| AB030 | None | 94 | ALIVE | Free | 0 | None | None |
| AB031 | None | 2 | DEAD | Free | 0 | Current | Current |
| AB032 | None | 15 | DEAD | Unknown | 0 | Current | Current |
| AB033 | Local | 7 | DEAD | Not free | 2B | Current | None |
| AB034 | None | 42 | DEAD | Free | 0 | Unknown | Unknown |
| AB035 | None | 48 | DEAD | Free | 0 | Unknown | None |
| AB037 | None | 27 | DEAD | Free | 1 | None | Current |
| AB038 | None | 13 | DEAD | Free | 2 | Past | Previous |
| AB039 | Local | 34 | ALIVE | Not free | 0 | Past | Current |
| AB040 | Unknown | 31 | ALIVE | Free | 2B | Current | Current |
| AB041 | None | 97 | ALIVE | Free | 0 | Current | None |
| AB042 | None | 88 | ALIVE | Free | 0 | Past | Previous |
| AB045 | None | 37 | DEAD | Free | 0 | None | None |
| AB047 | Never | 39 | DEAD | Not free | 0 | Unknown | Previous |
| AB048 | None | 79 | ALIVE | Free | N0 | None | None |
| AB049 | None | 70 | ALIVE | Free | 0 | Unknown | Unknown |
| AB051 | Local | 32 | DEAD | Not free | 1 | Current | Current |
| AB052 | Local | 15 | DEAD | Not free | 1 | None | None |
| AB054 | None | 11 | DEAD | Free | 1 | Current | None |
| AB055 | Lymph node | 35 | DEAD | Not free | 0 | Past | Current |
| | met | | | | | | |
| AB056 | None | 7 | DEAD | Free | 2B | Current | Current |
| AB059 | None | 76 | ALIVE | Free | 1 | Unknown | Unknown |
| AB060 | Unknown | 33 | ALIVE | Free | 2C | Current | Previous |
| AB061 | Unknown | 99 | ALIVE | Unknown | 0 | Unknown | Current |
| AB062 | None | 22 | DEAD | Free | 0 | Current | Current |
| AB063 | None | 74 | ALIVE | Free | 0 | Current | Previous |
| AB064 | None | 66 | DEAD | Free | 0 | None | Current |
| AB065 | None | 7 | DEAD | Free | 0 | None | None |
| AB066 | None | 56 | ALIVE | Free | 0 | Current | None |
| AB067 | None | 2 | DEAD | Free | 0 | None | Previous |
| AB068 | Distant met | 130 | ALIVE | Not free | 0 | None | None |
| AB069 | None | 79 | ALIVE | Free | 0 | Unknown | Unknown |
| AB070 | None | 3 | DEAD | Not free | 0 | Past | Previous |
| AB071 | None | 1 | DEAD | Free | 0 | Unknown | Unknown |
| AB073 | None | 5 | DEAD | Not free | 2 | Unknown | Unknown |
| AB076 | None | 50 | DEAD | Free | 0 | None | Previous |
| AB077 | None | 64 | ALIVE | Free | 0 | Current | Current |
| AB079 | None | 8 | DEAD | Free | 0 | None | None |
| AB080 | None | 37 | ALIVE | Free | 0 | Current | Previous |
| AB081 | None | 46 | DEAD | Free | 2C | Current | Previous |
| AB082 | None | 31 | DEAD | Free | 1 | Unknown | Unknown |
| AB083 | None | 13 | DEAD | Not free | N1 | Current | Current |
| AB084 | None | 60 | ALIVE | Free | 2B | None | None |
| AB085 | None | 11 | DEAD | Free | 0 | Unknown | Unknown |
| AB086 | None | 43 | ALIVE | Free | 0 | Current | Previous |

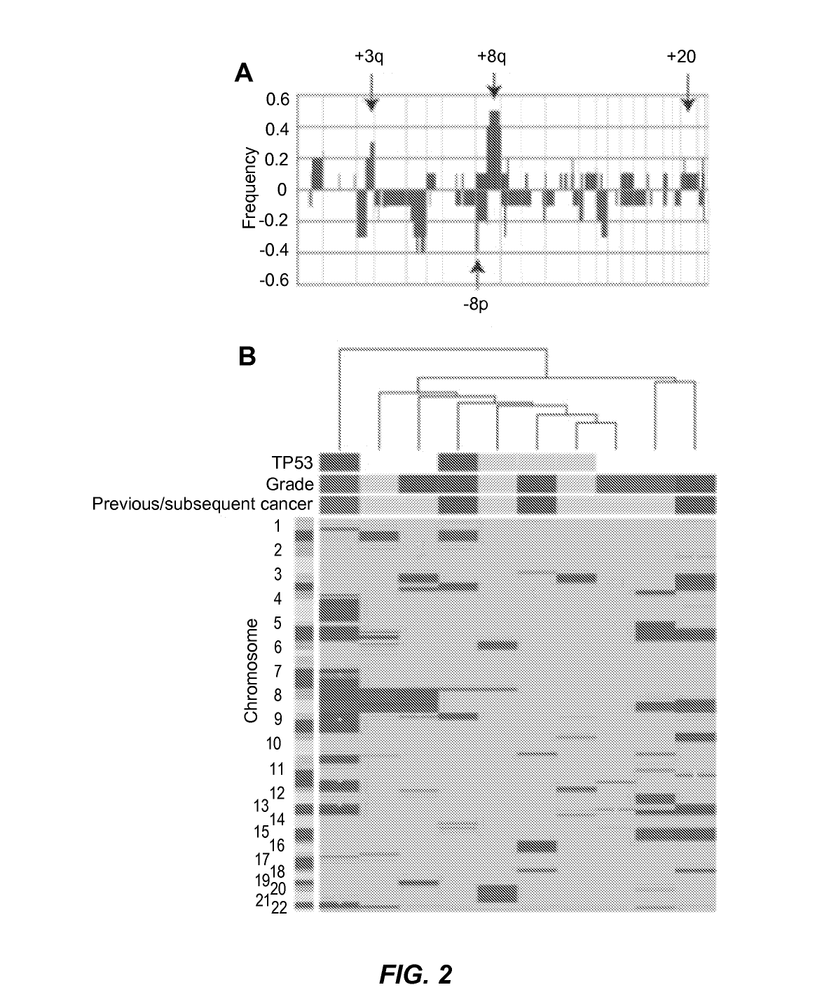

Considering the dysplasia cases with no known association with cancer, we found four regions of low level aberration (e.g. single copy gain and loss) that were each present in >20% of cases (FIG. 1A), including gains at 3q24-qter, 8q12-q24.2 and chromosome 20, and loss at 8pter-p23.1 (Table 4). The majority of the dysplasia cases (79%) harbored one or more of these recurrent aberrations, suggesting that these cases comprise a group, the 3q8pq20 subgroup, and the remaining 21% of cases, which lack +3q, −8p, +8q and +20, the non-3q8pq20 subgroup. Dysplasia grade and TP53 mutation status were not associated with subgroup membership (FIG. 1C). Further, analysis of a very limited number of dysplasia cases (n=10) that progressed to cancer or arose at the site of a previously treated cancer revealed that 3q8pq20 and non-3q8pq20 subtypes were present in similar proportions as in the dysplasia cohort with no known association with cancer, 70% and 30%, respectively (FIGS. 2A-B).

TABLE 4
Recurrent regions of aberration at ≧20% frequency in dysplasia with no
known association with cancer

| | Aber | | | Proximal | | Distal | | Max. |
|---|---|---|---|---|---|---|---|---|
| Region | ration | Start kb | End kb | clone | Marker | clone | Marker | Freq. |
| 3q24-qter | Gain | 146541.915 | 199505.74 | RP11- | AFM | GS1- | | 0.41 |
| | | | | 72E23 | 210VE7 | 56H22 | | |
| 8pter-p23.1 | Loss | 0.001 | 10893.274 | GS1- | | RP11- | SHGC- | 0.34 |
| | | | | 77L23 | | 252K12 | 1962 | |
| 8q12-q24.2 | Gain | 61264.084 | 134076.075 | RP11- | SHGC- | RP11- | SHGC- | 0.52 |
| | | | | 258B14 | 32354 | 184M21 | 1948 | |
| 20pter-qter | Gain | 0.001 | 62435.964 | RP1- | | RP1- | | 0.28 |
| | | | | 82O2 | | 81F12 | | |

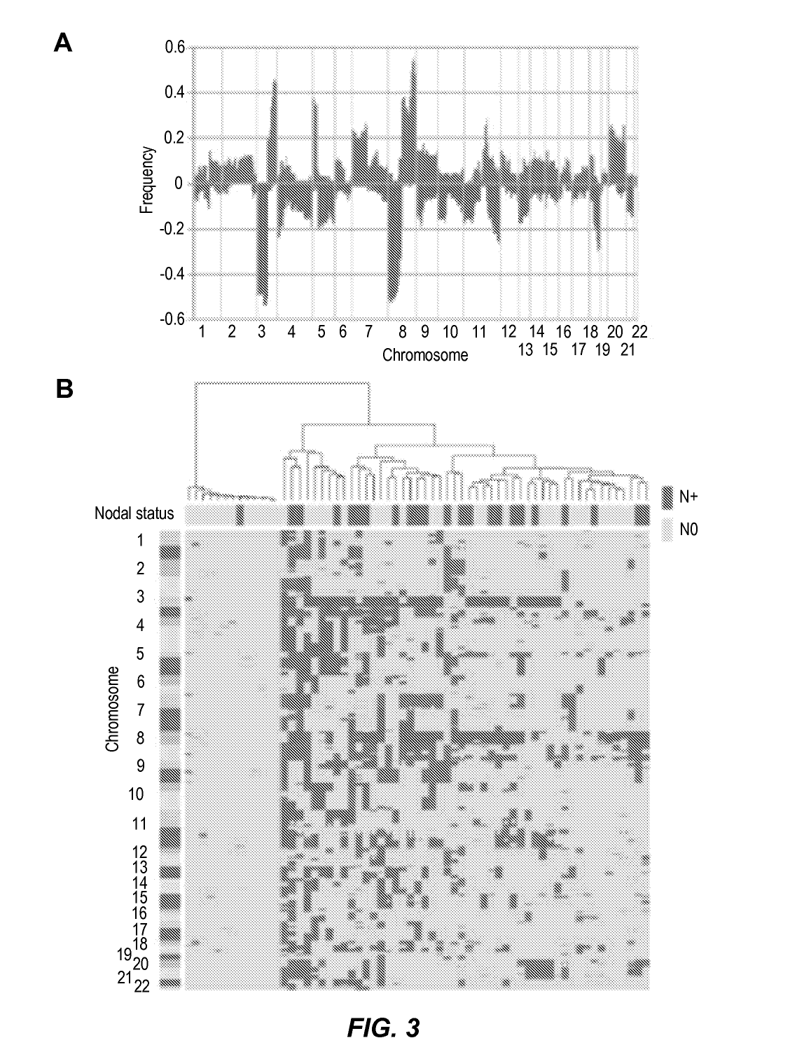

We noted that gains of 3q, 8q, 20 and loss of 8p were frequent aberrations in both oral SCC cohorts (FIGS. 1B and D, FIG. 3) and the frequency did not differ from that in the dysplasia cases (FIG. 1E, Table 5). Moreover, the frequency of tumors harboring one or more of these aberrations was not significantly different than the frequency in dysplasia (FIG. 1F, 67% and 76%, p=0.25 and p=0.8 for SCC cohort#1 and cohort#2, respectively), suggesting that not only dysplasias, but also oral SCCs can be assigned to 3q8pq20 and non-3q8pq20 subtypes.

TABLE 5
Frequency in dysplasia and SCC cohorts of copy number changes occurring in ≧20% of dysplasia with no known association with cancer

| | | | | | Dysplasia with | | | | | | | | | | | |
|---|---|---|---|---|---|---|---|---|---|---|---|---|---|---|---|---|
| | Dysplasia | | | | cancer | | | | cohort#1 | | | | cohort#2 | | | |
| | n = 29 | | | | n = 10 | | | | n = 89 | | | | n = 63 | | | |
| | 3q24- | 8pter- | 8q12- | | 3q24- | 8pter- | 8q12- | | 3q24- | 8pter- | 8q12- | 20pter- | 3q24- | 8pter- | 8q12- | 20pter- |
| | qter | p23-1 | q24.2 | 20pter-qter | qter | p23-1 | q24.2 | 20pter-qter | qter | p23-1 | q24.2 | qter | qter | p23-1 | q24.2 | qter |
| Frequency | 0.41 | 0.34 | 0.52 | 0.28 | 0.3 | 0.4 | 0.5 | 0.2 | 0.25 | 0.35 | 0.37 | 0.17 | 0.46 | 0.52 | 0.56 | 0.25 |
| Dysplasia | | | | | | | | | | | | | | | | |
| p-value | | | | | 0.48 | 0.70 | 1.00 | 0.69 | 0.09 | 1.00 | 0.17 | 0.16 | 0.68 | 0.07 | 0.69 | 0.82 |
| odds ratio | | | | | 1.87 | 0.68 | 1.07 | 1.77 | 0.47 | 1.07 | 0.56 | 0.52 | 1.22 | 2.18 | 1.19 | 0.87 |
| lower CI | | | | | 0.34 | 0.12 | 0.20 | 0.27 | 0.20 | 0.45 | 0.24 | 0.19 | 0.51 | 0.89 | 0.49 | 0.32 |
| upper CI | | | | | 13.44 | 4.13 | 5.79 | 20.46 | 1.14 | 2.60 | 1.29 | 1.41 | 2.99 | 5.53 | 2.85 | 2.38 |
| Dysplasia with | | | | | | | | | | | | | | | | |
| cancer | | | | | | | | | | | | | | | | |
| p-value | | | | | | | | | 0.71 | 0.74 | 0.50 | 0.68 | 0.50 | 0.52 | 0.75 | 1.00 |
| odds ratio | | | | | | | | | 0.77 | 0.80 | 0.59 | 0.81 | 1.97 | 1.64 | 1.25 | 1.36 |
| lower CI | | | | | | | | | 0.16 | 0.18 | 0.13 | 0.14 | 0.40 | 0.35 | 0.26 | 0.23 |
| upper CI | | | | | | | | | 5.00 | 4.17 | 2.78 | 8.61 | 12.89 | 8.69 | 6.01 | 14.42 |

To confirm that the frequencies of the two subtypes were not simply a characteristic of oral cancers from Northern California, we accessioned an independent oral SCC array CGH dataset from the Netherlands (Smeets et al. 2009) comprised of 29 cases. We did not find a significant difference in the proportion of 3q8pq20 and non-3q8pq20 subtypes (75% and 25%, respectively; p=0.76) among the 28 cases with copy number data of sufficient quality. Moreover, since these 28 cases had tested negative for human papillomavirus (HPV), these observations allow us to rule out HPV infection, which is a common etiologic agent in oropharyngeal cancers, but not oral cavity cancers (Herrero et al. 2003), as an underlying determinant of subtype. Thus, 3q8pq20 and non-3q8pq20 subtypes and their relative proportions appear to be a universal feature of oral SCC cases from western countries.

Although dysplasia and oral SCC share recurrent aberrations involving 3q, 8p, 8q and chromosome 20, it is clear from FIG. 1, that copy number aberrations are more frequent in oral SCCs. For example, in the 89 SCCs of cohort#1, 11 aberrant loci that occurred in ≧15% of cases included −3p, −4-p, −4-q, +5p, −5q, +7p, −9p, +11q13, −18q, −21q and a loss at 8p12 that maps proximal to the region of loss at 8p shared by dysplasia and SCC (FIG. 1B, Table 6). Therefore, to identify copy number alterations that might distinguish pre-cancers and cancers, we first defined recurrent gains and losses as those occurring at >20% frequency in SCC cohort#2, and then compared the frequency of recurrent aberrations in all 39 dysplasia cases to those in the independent SCC cohort#1 This analysis found only the region +7pter-p11.2 (q-value=0.036) to be significantly more frequent in cancers (Table 7), suggesting that up-regulation of gene(s) in this region may occur late in progression to cancer.

TABLE 6
Regions altered in ≧15% of SCC cases in cohort#1

| | | | | Proximal | | Distal | | Maximum |
|---|---|---|---|---|---|---|---|---|
| Region | Aberration | Start bp | End bp | Clone | Marker | Clone | Marker | Frequency |
| 3pter-p14.1 | Loss | 1 | 71,540,269 | CTB-228K22 | | RP11-154H23 | AFMA176YG9; | 0.28 |
| | | | | | | | D3S3568 | |
| 3q24-qter | Gain | 145,059,218 | 198,022,429 | RP11-72E23 | AFM210VE7; D3S1557 | GS1-56H22 | | 0.25 |
| 4p15.3-p15.2 | Loss | 19,436,965 | 24,135,167 | RP11-11M9 | SHGC4-737; L09901.1 | RP11-276O17 | AFM158XC7; | 0.15 |
| | | | | | | | D4S404 | |
| 4q33-4q35 | Loss | 172,302,780 | 182,554,327 | RP11-272N13 | SHGC4-612; Z23484 | RP11-125M9 | SHGC-24974; | 0.15 |
| | | | | | | | G33820 | |
| 5pter-p13.2 | Gain | 1 | 37,847,186 | RP1-24H17 | | RP11-253B9 | AFM155XH12; | 0.17 |
| | | | | | | | D5S1964 | |
| 5q12-q23 | Loss | 62,765,715 | 128,152,700 | RP11-174I22 | AFM238XA3; D5S427 | RP11-45L19 | AFM286XG9; | 0.17 |
| | | | | | | | D5S642 | |
| 7p11.2-p12.1 | Gain | 54,986,820 | 55,628,978 | RP11-14K11 | AFM102YA1; D7S2550 | RP11-34J24 | SHGC-32070; | 0.16 |
| | | | | | | | Z43514 | |
| 8p23.3-p21.2 | Loss | 2,070,529 | 22,554,607 | RP11-117P11 | SHGC-9645; G11277 | RP11-274M9 | SHGC-36225; | 0.35 |
| | | | | | | | R68283 | |
| 8p12 | Loss | 31,206,638 | 32,412,400 | CTD-2020E14 | WRN | RP11-57I3 | SHGC-894; Z16888 | 0.15 |
| 8q11.1-qter | Gain | 47,805,281 | 146,364,021 | RP11-12L15 | SHGC-15321; G13932 | GS1-261I1 | | 0.37 |
| 9pter-p21.1 | Loss | 1 | 31,186,858 | CTB-41L13 | | RP11-70F16 | SHGC-6958; | 0.21 |
| | | | | | | | M98789.1 | |
| 11q13-q13.4 | Gain | 64,538,329 | 70,403,075 | CTD-2220I9 | A007D15; D11S4946 | RP11-120P20 | SHGC-4518; | 0.24 |
| | | | | | | | L06492.1 | |
| 18q22-qter | Loss | 45,371,283 | 78,077,247 | RP11-748M14 | R77259; SMAD2 | RP11-507P3 | | 0.18 |
| 20pter-p13 | Gain | 1 | 5,517,913 | RP1-82O2 | | RP11-149O7 | AFMB290WH5; | 0.17 |
| | | | | | | | D20S882 | |
| 20p12.2 | Gain | 10,286,846 | 11,075,800 | RMC20P160 | WI-7829 | RP11-60N17 | AFM292XB5; | 0.16 |
| | | | | | | | D20S189 | |
| 21q21.3 | Gain | 23,303,087 | 25,112,561 | RP11-86J21 | AFMA081WF1; D21S1918 | RP11-13J15 | SHGC-4988; | 0.15 |
| | | | | | | | L16389.1 | |

Start and end positions determined by mapping of BAC clone or STS marker according to February 2009 (hg19) assembly positions. Positions of telomeric clones assigned as first or last base pair according to the February 2009 assembly.

TABLE 7
Frequency of recurrent common gains or losses in all dysplasia cases and oral SCC cohort#1

| | Chromosome | | | | | |
|---|---|---|---|---|---|---|
| | 3 | 3 | 4 | 4 | 5 | 7 |
| start position (KB) | 0.001 | 118631.1 | 4538.586 | 23220.448 | 0.001 | 0.001 |
| end position (KB) | 100971.51 | 199505.74 | 9681.528 | 33086.961 | 41319.671 | 55381.621 |
| type of copy number | loss | gain | loss | loss | gain | gain |
| alteration | | | | | | |
| Adjusted p-value | 0.3046572 | 0.3624367 | 1 | 0.6133253 | 0.4991492 | 0.0357378 |
| Raw p-value | 0.0834298 | 0.1393987 | 1 | 0.4246098 | 0.2687727 | 0.0027491 |
| No. Present All Cases | 128 | 128 | 128 | 128 | 128 | 128 |
| No. Gain All Cases | 0 | 37 | 0 | 0 | 17 | 16 |
| No. Lost All cases | 33 | 0 | 12 | 19 | 0 | 0 |
| Proportion Present All Cases | 1 | 1 | 1 | 1 | 1 | 1 |
| Proportion Gained All Cases | 0 | 0.29 | 0 | 0 | 0.13 | 0.12 |
| Proportion Lost All Cases | 0.26 | 0 | 0.09 | 0.15 | 0 | 0 |
| No. Present Dysplasia | 39 | 39 | 39 | 39 | 39 | 39 |
| No. Gain Dysplasia | 0 | 15 | 0 | 0 | 3 | 0 |
| No. Lost Dysplasia | 6 | 0 | 3 | 4 | 0 | 0 |
| Proportion Present Dysplasia | 1 | 1 | 1 | 1 | 1 | 1 |
| Proportion Gained Dysplasia | 0 | 0.38 | 0 | 0 | 0.08 | 0 |
| Proportion Lost Dysplasia | 0.15 | 0 | 0.08 | 0.1 | 0 | 0 |
| No. Present SCC cohort#1 | 89 | 89 | 89 | 89 | 89 | 89 |
| No. Gain SCC cohort#1 | 0 | 22 | 0 | 0 | 14 | 16 |
| No. Lost SCC cohort#1 | 27 | 0 | 9 | 15 | 0 | 0 |
| Proportion Present SCC cohort#1 | 1 | 1 | 1 | 1 | 1 | 1 |
| Proportion Gained SCC cohort#1 | 0 | 0.25 | 0 | 0 | 0.16 | 0.18 |
| Proportion Lost SCC cohort#1 | 0.3 | 0 | 0.1 | 0.17 | 0 | 0 |

| | Chromosome | | | | | | |
|---|---|---|---|---|---|---|---|
| | 8 | 8 | 11 | 11 | 18 | 20 | 20 |
| start position (KB) | 0.001 | 47924.445 | 69070.147 | 101922.84 | 42182.657 | 0.001 | 51601.945 |
| end position (KB) | 43102.83 | 146274.83 | 71610.465 | 134452.38 | 76117.153 | 35483.684 | 52203.394 |
| type of copy number | loss | gain | gain | loss | loss | gain | gain |
| alteration | | | | | | | |
| Adjusted p-value | 1 | 0.3738449 | 0.3046572 | 1 | 0.3046572 | 1 | 0.6133253 |
| Raw p-value | 1 | 0.1725438 | 0.0937407 | 1 | 0.058666 | 0.8249574 | 0.4167683 |
| No. Present All Cases | 128 | 128 | 128 | 128 | 128 | 128 | 128 |
| No. Gain All Cases | 0 | 53 | 25 | 0 | 0 | 31 | 18 |
| No. Lost All cases | 45 | 0 | 0 | 11 | 18 | 0 | 0 |
| Proportion Present All Cases | 1 | 1 | 1 | 1 | 1 | 1 | 1 |
| Proportion Gained All Cases | 0 | 0.41 | 0.2 | 0 | 0 | 0.24 | 0.14 |
| Proportion Lost All Cases | 0.35 | 0 | 0 | 0.09 | 0.14 | 0 | 0 |
| No. Present Dysplasia | 39 | 39 | 39 | 39 | 39 | 39 | 39 |
| No. Gain Dysplasia | 0 | 20 | 4 | 0 | 0 | 10 | 7 |
| No. Lost Dysplasia | 14 | 0 | 0 | 3 | 2 | 0 | 0 |
| Proportion Present Dysplasia | 1 | 1 | 1 | 1 | 1 | 1 | 1 |
| Proportion Gained Dysplasia | 0 | 0.51 | 0.1 | 0 | 0 | 0.26 | 0.18 |
| Proportion Lost Dysplasia | 0.36 | 0 | 0 | 0.08 | 0.05 | 0 | 0 |
| No. Present SCC cohort#1 | 89 | 89 | 89 | 89 | 89 | 89 | 89 |
| No. Gain SCC cohort#1 | 0 | 33 | 21 | 0 | 0 | 21 | 11 |
| No. Lost SCC cohort#1 | 31 | 0 | 0 | 8 | 16 | 0 | 0 |
| Proportion Present SCC | 1 | 1 | 1 | 1 | 1 | 1 | 1 |
| cohort#1 | | | | | | | |
| Proportion Gained SCC | 0 | 0.37 | 0.24 | 0 | 0 | 0.24 | 0.12 |
| cohort#1 | | | | | | | |
| Proportion Lost SCC | 0.35 | 0 | 0 | 0.09 | 0.18 | 0 | 0 |
| cohort#1 | | | | | | | |

Copy Number Aberrations are More Frequent in the 3q8pq20 Subtype

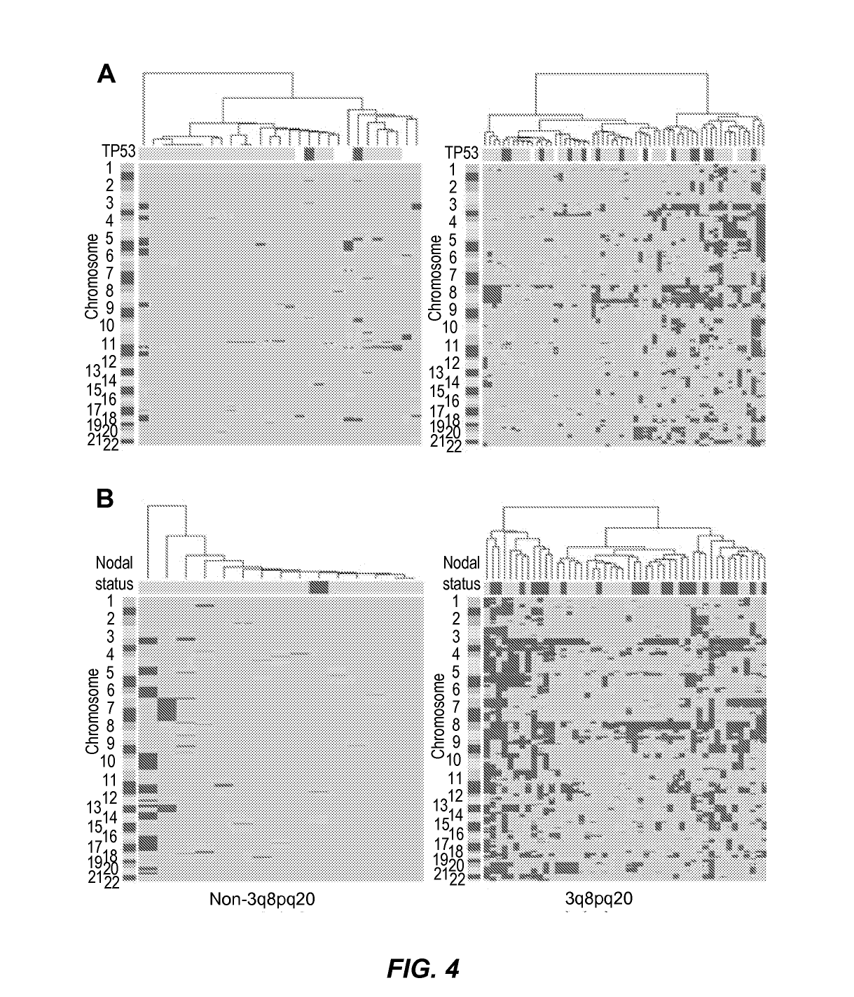

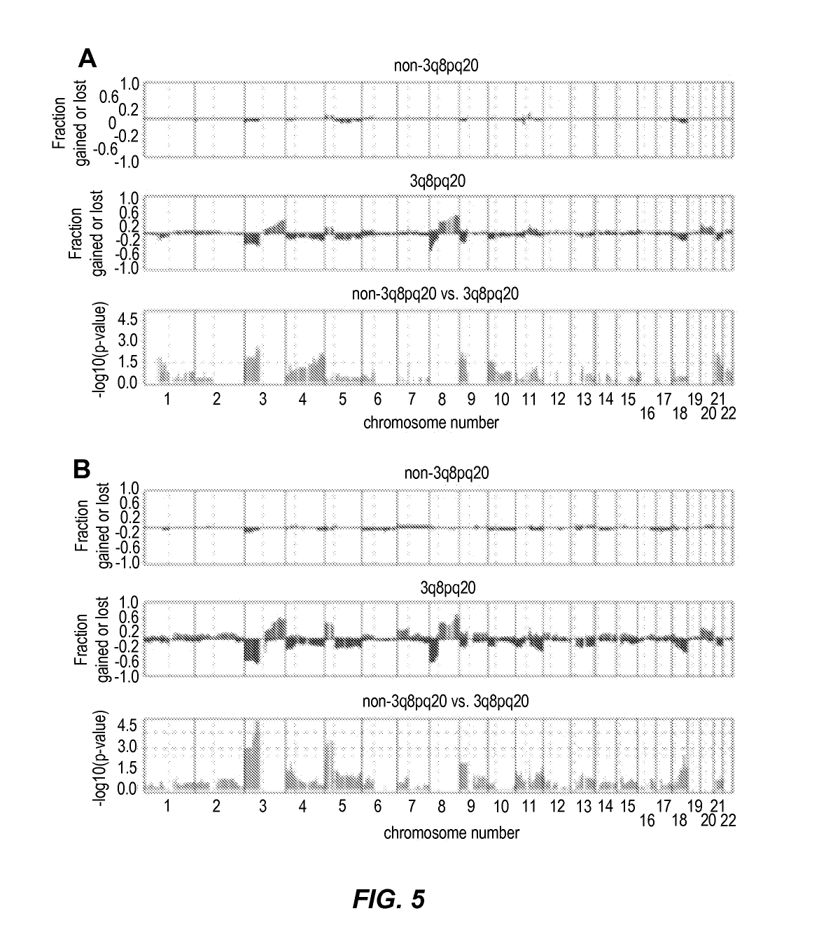

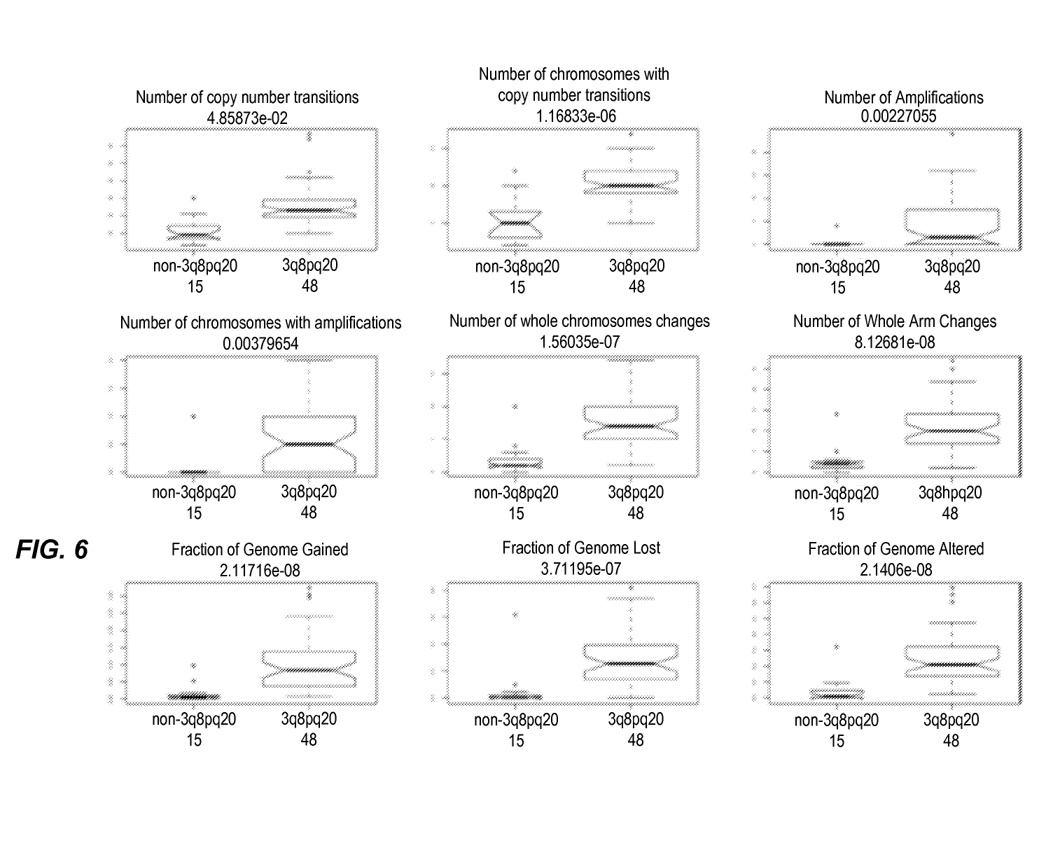

Hierarchical clustering of the cases in the two oral SCC cohorts revealed that recurrent low level gains and losses were not uniformly distributed (FIG. 1D and FIG. 3). Indeed, we observed that recurrent aberrations were more frequent in the 3q8pq20 subtype, which also further subdivides into high and low instability tumors (FIG. 4, FIG. 5). In addition, we observed a highly significant association of 3q8pq20 subtype with various types of chromosomal level genome instability (FIG. 6), including, for example, differences in the fraction of the genome gained (p<10-9), lost (p<10-6) and altered (p<10-8). On the other hand, although we more frequently observed mutations in exons 5-8 of TP53 (often associated with higher levels of genome instability) in the 3q8pq20 group of cohort#1 compared to the non-3q8pq20 group, the difference was not significant (Fisher's exact test, p=0.12).

The 3q8pq20 Tumors with High Levels of Chromosomal Instability are Differentially Methylated

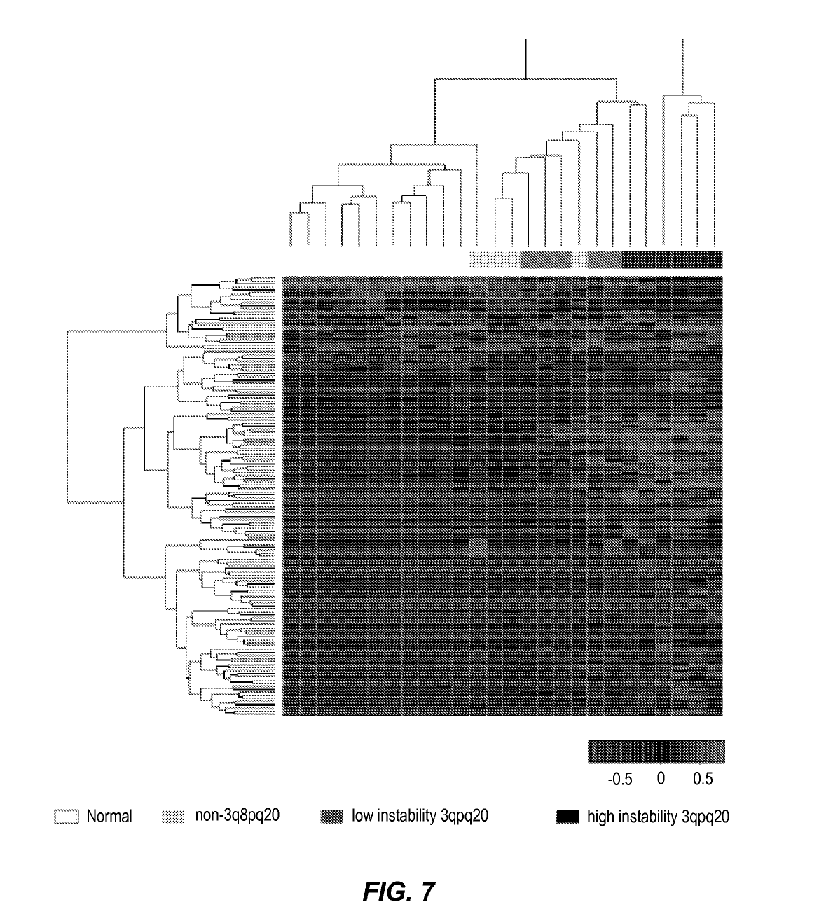

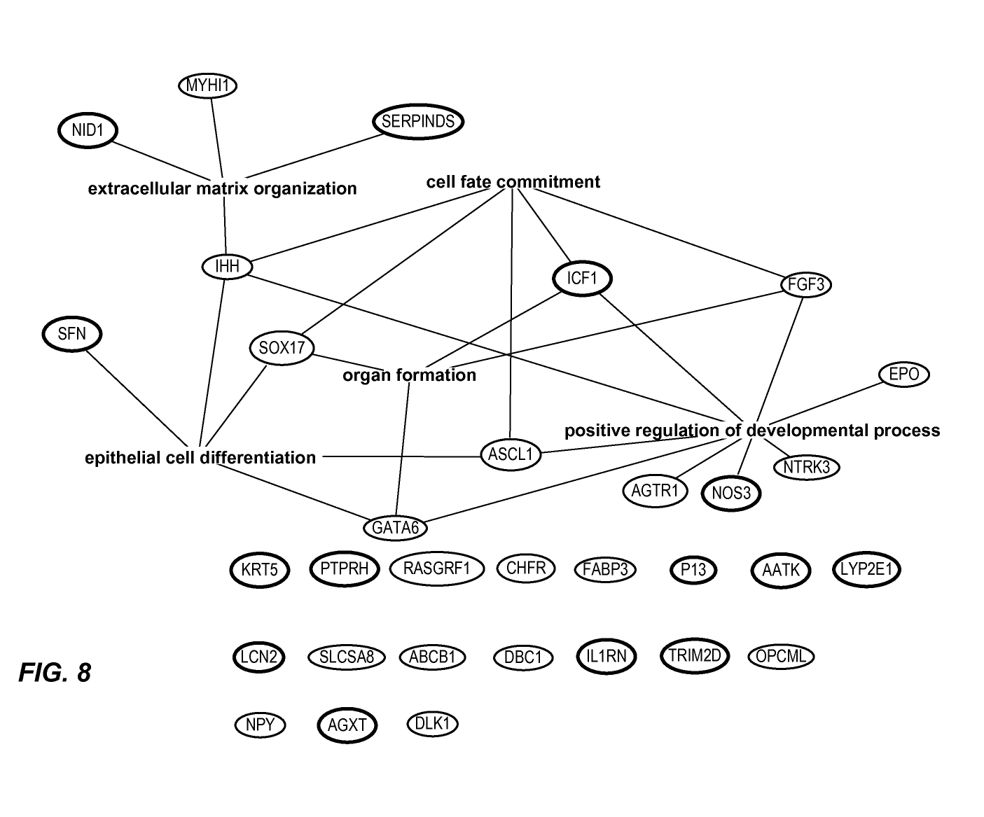

The lack of chromosome level instability in non-3q8pq20 tumors suggests that development of these tumors could be associated with other, copy number neutral, mechanisms, such as microsatellite instability or epigenetic alterations. Microsatellite instability is not common in oral cancer (Shaw et al. 2008), whereas genome-wide alterations in methylation patterns are observed (Poage et al. 2010). Therefore, to investigate whether 3q8pq20 and non-3q8pq20 oral SCC subtypes differed in methylation patterns, we accessioned a published dataset for a head and neck cancer patient cohort comprised of 15 oral cavity and 4 oropharyngeal tumors (Poage et al. 2010) for which both copy number and methylation measurements were available (NCBI GEO accession GSE20939). We assigned 3q8pq20 status to the oral cavity cases (Table 8). Hierarchical clustering using the top 10% most variable methylation probes (142 probes, Table 9) revealed that differential methylation was associated with the cases with the greater number of copy number alterations (high 3q8pq20), as noted previously (Poage et al. 2010). The highly unstable 3q8pq20 cases clustered separately from the low genomic instability 3q8pq20, non-3q8pq20 and normal samples (FIG. 7). The normal control cases also clustered together, whereas the non-3q8pq20 and low instability 3q8pq20 cases were somewhat intermixed, suggesting that extensive epigenetic alterations do not contribute to formation of non-3q8pq20 tumors. For the highly unstable 3q8pq20 cases, we identified 37 differentially methylated probes representing 32 genes (Table 10), with significant enrichment for Gene Ontology processes involving four or more of these genes (p<0.02); organ formation, epithelial cell differentiation, extracellular matrix organization, cell fate commitment, and positive regulation of developmental process (Table 11 and FIG. 8).

TABLE 8
Patient characteristics and 3q8pq20 status for cases reported by Poage et al. 2010 (NCBI GEO Accession GSE20939)

| Sample Name | Published Sample | Methylation data | | | | | |
|---|---|---|---|---|---|---|---|
| (GEO) | Name* | (GEO accession) | Sample type | Tumor type | Age | Stage | Gender |
| Tumor_1 | 101 | GSM520573 | oral | 3q8pq20 - Low instabiity | 57 | | F |
| Tumor_2 | 113 | GSM520574 | oral | 3q8pq20 - Low instabiity | 85 | 1 | F |
| Tumor_3 | 117 | GSM520575 | oral | 3q8pq20 - High instability | 48 | 4 | M |
| Tumor_4 | 111 | GSM520576 | oral | 3q8pq20 - High instability | 57 | 3 | M |
| Tumor_5 | 112 | GSM520577 | oral | 3q8pq20 - Low instabiity | 50 | 4 | M |
| Tumor_6 | 107 | GSM520578 | oral | 3q8pq20 - Low instabiity | 50 | 4 | M |
| Tumor_7 | 114 | GSM520579 | oral | 3q8pq20 - High instability | 50 | 4 | M |
| Tumor_8 | 119 | GSM520580 | larynx | Not determined | 84 | 4 | M |
| Tumor_9 | 106 | GSM520581 | oral | non-3q8pq20 | 67 | 3 | M |
| Tumor_10 | 118 | GSM520582 | pharynx | Not determined | 57 | 3 | M |
| Tumor_11 | 116 | GSM520583 | oral | non-3q8pq20 | 74 | 2 | M |
| Tumor_12 | 102 | GSM520584 | oral | 3q8pq20 - High instability | 46 | 2 | M |
| Tumor_13 | 109 | GSM520585 | oral | 3q8pq20 - Low instabiity | 77 | 4 | M |
| Tumor_14 | 104 | GSM520586 | larynx | Not determined | 67 | 1 | M |
| Tumor_15 | 110 | GSM520587 | pharynx | Not determined | 70 | 3 | M |
| Tumor_16 | 103 | GSM520588 | oral | non-3q8pq20 | 49 | 4 | M |
| Tumor_17 | 115 | GSM520589 | oral | non-3q8pq20 | 25 | 4 | F |
| Tumor_18 | 105 | GSM520590 | oral | 3q8pq20 - High instability | 45 | 3 | M |
| Tumor_19 | 108 | GSM520591 | oral | 3q8pq20 - High instability | 54 | 4 | F |

*From Poage et al., 2010, PLoS ONE 5(3): e9651

TABLE 9
Most variable methylation probes (variance above the 90th percentile) from Poage et
al. 2010 (NCBI GEO Accession GSE20939)

| | GenBank | | | | |
|---|---|---|---|---|---|
| Probe ID | Accession | GI | EntrezGene ID | Gene | Std Dev |
| ABCC2_E16_R | NM_000392.1 | 4557480 | 1244 | ABCC2 | 0.329555 |
| ADCYAP1_P398_F | NM_001117.2 | 10947062 | 116 | ADCYAP1 | 0.396472 |
| ADCYAP1_P455_R | NM_001117.2 | 10947062 | 116 | ADCYAP1 | 0.400806 |
| AGTR1_P154_F | NM_000685.3 | 14043060 | 185 | AGTR1 | 0.387685 |
| AGTR1_P41_F | NM_000685.3 | 14043060 | 185 | AGTR1 | 0.484838 |
| AGXT_P180_F | NM_000030.1 | 4557288 | 189 | AGXT | 0.348522 |
| AIM2_P624_F | NM_004833.1 | 4757733 | 9447 | AIM2 | 0.395463 |
| ASCL1_E24_F | NM_004316.2 | 55743093 | 429 | ASCL1 | 0.359431 |
| BDNF_P259_R | NM_170733.2 | 34106708 | 627 | BDNF | 0.330396 |
| BMP3_P56_R | NM_001201.1 | 4557370 | 651 | BMP3 | 0.306269 |
| CALCA_E174_R | NM_001033952.1 | 76880483 | 796 | CALCA | 0.406913 |
| CCKBR_P480_F | NM_176875.2 | 33356159 | 887 | CCKBR | 0.313604 |
| CCNA1_E7_F | NM_003914.2 | 16306528 | 8900 | CCNA1 | 0.499197 |
| CDKN1A_P242_F | NM_000389.2 | 17978496 | 1026 | CDKN1A | 0.412829 |
| CHFR_P501_F | NM_018223.1 | 8922674 | 55743 | CHFR | 0.463364 |
| CHGA_E52_F | NM_001275.2 | 10800418 | 1113 | CHGA | 0.373197 |
| COL1A1_P5_F | NM_000088.2 | 14719826 | 1277 | COL1A1 | 0.324329 |
| CSF1R_E26_F | NM_005211.2 | 27262658 | 1436 | CSF1R | 0.310113 |
| CYP1B1_E83_R | NM_000104.2 | 13325059 | 1545 | CYP1B1 | 0.335418 |
| CYP2E1_P416_F | NM_000773.3 | 75709190 | 1571 | CYP2E1 | 0.322617 |
| DAPK1_P345_R | NM_004938.1 | 4826683 | 1612 | DAPK1 | 0.321537 |
| DBC1_E204_F | NM_014618.1 | 7657008 | 1620 | DBC1 | 0.385207 |
| DBC1_P351_R | NM_014618.1 | 7657008 | 1620 | DBC1 | 0.378113 |
| DCC_P471_R | NM_005215.1 | 4885174 | 1630 | DCC | 0.355517 |
| DLC1_E276_F | NM_182643.1 | 33188432 | 10395 | DLC1 | 0.339272 |
| DLK1_E227_R | NM_003836.4 | 74136022 | 8788 | DLK1 | 0.439204 |
| EPHA5_E158_R | NM_182472.1 | 32967318 | 2044 | EPHA5 | 0.305924 |
| EPO_E244_R | NM_000799.2 | 62240996 | 2056 | EPO | 0.518786 |
| EPO_P162_R | NM_000799.2 | 62240996 | 2056 | EPO | 0.343503 |
| EYA4_E277_F | NM_004100.2 | 26667248 | 2070 | EYA4 | 0.47034 |
| FABP3_E113_F | NM_004102.3 | 62865867 | 2170 | FABP3 | 0.322652 |
| FANCE_P356_R | NM_021922.2 | 66879667 | 2178 | FANCE | 0.319235 |
| FGF12_P210_R | NM_021032.2 | 21614509 | 2257 | FGF12 | 0.354068 |
| FGF3_E198_R | NM_005247.2 | 15451899 | 2248 | FGF3 | 0.363928 |
| FGF3_P171_R | NM_005247.2 | 15451899 | 2248 | FGF3 | 0.308145 |
| FGF5_P238_R | NM_004464.3 | 73486654 | 2250 | FGF5 | 0.302008 |
| FLI1_E29_F | NM_002017.2 | 7110592 | 2313 | FLI1 | 0.326201 |
| FLT1_P615_R | NM_002019.2 | 32306519 | 2321 | FLT1 | 0.311719 |
| FLT3_E326_R | NM_004119.1 | 4758395 | 2322 | FLT3 | 0.435187 |
| FLT3_P302_F | NM_004119.1 | 4758395 | 2322 | FLT3 | 0.353058 |
| GAS7_E148_F | NM_003644.2 | 41406075 | 8522 | GAS7 | 0.398224 |
| GAS7_P622_R | NM_003644.2 | 41406075 | 8522 | GAS7 | 0.393379 |
| GATA6_P726_F | NM_005257.3 | 40288196 | 2627 | GATA6 | 0.356988 |
| GFI1_P208_R | NM_005263.2 | 71037376 | 2672 | GFI1 | 0.351948 |
| GP1BB_E23_F | NM_000407.3 | 9945387 | 2812 | GP1BB | 0.342825 |
| GP1BB_P278_R | NM_000407.3 | 9945387 | 2812 | GP1BB | 0.306203 |
| H19_P541_F | NR_002196.1 | 57862814 | 283120 | H19 | 0.319096 |
| HOXA11_E35_F | NM_005523.4 | 24497552 | 3207 | HOXA11 | 0.308459 |
| HOXA11_P698_F | NM_005523.4 | 24497552 | 3207 | HOXA11 | 0.395211 |
| HOXA5_E187_F | NM_019102.2 | 24497516 | 3202 | HOXA5 | 0.305959 |
| HOXA5_P1324_F | NM_019102.2 | 24497516 | 3202 | HOXA5 | 0.327445 |
| HOXA9_E252_R | NM_002142.3 | 24497558 | 3205 | HOXA9 | 0.432456 |
| HOXA9_P1141_R | NM_002142.3 | 24497558 | 3205 | HOXA9 | 0.48369 |
| HOXB13_E21_F | NM_006361.4 | 70167332 | 10481 | HOXB13 | 0.349827 |
| HOXB13_P17_R | NM_006361.4 | 70167332 | 10481 | HOXB13 | 0.429117 |
| HS3ST2_E145_R | NM_006043.1 | 5174462 | 9956 | HS3ST2 | 0.435634 |
| HS3ST2_P171_F | NM_006043.1 | 5174462 | 9956 | HS3ST2 | 0.411331 |
| HTR1B_E232_R | NM_000863.1 | 4504532 | 3351 | HTR1B | 0.329201 |
| HTR1B_P107_F | NM_000863.1 | 4504532 | 3351 | HTR1B | 0.36197 |
| HTR1B_P222_F | NM_000863.1 | 4504532 | 3351 | HTR1B | 0.51574 |
| ICAM1_P386_R | NM_000201.1 | 4557877 | 3383 | ICAM1 | 0.302438 |
| IGF2AS_P203_F | NM_016412.1 | 7705972 | 51214 | IGF2AS | 0.318319 |
| IGSF4_P86_R | NM_014333.2 | 22095346 | 23705 | IGSF4 | 0.347705 |
| IHH_E186_F | NM_002181.1 | 51467740 | 3549 | IHH | 0.37671 |
| IL12B_P392_R | NM_002187.2 | 24497437 | 3593 | IL12B | 0.406301 |
| IRAK3_E130_F | NM_007199.1 | 6005791 | 11213 | IRAK3 | 0.311449 |
| IRAK3_P13_F | NM_007199.1 | 6005791 | 11213 | IRAK3 | 0.410585 |
| IRAK3_P185_F | NM_007199.1 | 6005791 | 11213 | IRAK3 | 0.383186 |
| ISL1_P554_F | NM_002202.1 | 4504736 | 3670 | ISL1 | 0.305391 |
| JAK3_E64_F | NM_000215.2 | 47157314 | 3718 | JAK3 | 0.350375 |
| JAK3_P156_R | NM_000215.2 | 47157314 | 3718 | JAK3 | 0.389731 |
| LTA_E28_R | NM_000595.2 | 6806892 | 4049 | LTA | 0.333609 |
| LY6G6E_P45_R | NM_024123.1 | 13236491 | 79136 | LY6G6E | 0.303038 |
| MAP3K9_E17_R | NM_033141.2 | 52421789 | 4293 | MAP3K9 | 0.353886 |
| MAPK10_E26_F | NM_138982.1 | 20986509 | 5602 | MAPK10 | 0.352773 |
| MDR1_seq_42_S300_R | NM_000927.3 | 42741658 | 5243 | ABCB1 | 0.460199 |
| MME_E29_F | NM_000902.2 | 6042205 | 4311 | MME | 0.437296 |
| MME_P388_F | NM_000902.2 | 6042205 | 4311 | MME | 0.32345 |
| MMP2_P303_R | NM_004530.2 | 75905807 | 4313 | MMP2 | 0.342015 |
| MMP3_P16_R | NM_002422.3 | 73808272 | 4314 | MMP3 | 0.409868 |
| MMP9_P189_F | NM_004994.2 | 74272286 | 4318 | MMP9 | 0.313326 |
| MOS_E60_R | NM_005372.1 | 4885488 | 4342 | MOS | 0.468389 |
| MT1A_E13_R | NM_005946.2 | 71274112 | 4489 | MT1A | 0.430838 |
| MT1A_P49_R | NM_005946.2 | 71274112 | 4489 | MT1A | 0.474958 |
| MYH11_P22_F | NM_022844.1 | 13124874 | 4629 | MYH11 | 0.405195 |
| MYOD1_E156_F | NM_002478.3 | 23111008 | 4654 | MYOD1 | 0.390355 |
| NEFL_E23_R | NM_006158.1 | 5453761 | 4747 | NEFL | 0.453197 |
| NID1_P677_F | NM_002508.1 | 4505394 | 4811 | NID1 | 0.342524 |
| NPY_E31_R | NM_000905.2 | 31542152 | 4852 | NPY | 0.378115 |
| NPY_P295_F | NM_000905.2 | 31542152 | 4852 | NPY | 0.478911 |
| NTRK1_E74_F | NM_001007792.1 | 56118209 | 4914 | NTRK1 | 0.397523 |
| NTRK3_E131_F | NM_002530.2 | 59889559 | 4916 | NTRK3 | 0.338216 |
| NTRK3_P636_R | NM_002530.2 | 59889559 | 4916 | NTRK3 | 0.4058 |
| OPCML_E219_R | NM_002545.3 | 59939898 | 4978 | OPCML | 0.388809 |
| OSM_P188_F | NM_020530.3 | 28178862 | 5008 | OSM | 0.350721 |
| OSM_P34_F | NM_020530.3 | 28178862 | 5008 | OSM | 0.307398 |
| p16_seq_47_S188_R | NM_058195.2 | 47132605 | 1029 | CDKN2A | 0.483742 |
| PDGFB_E25_R | NM_002608.1 | 4505680 | 5155 | PDGFB | 0.30496 |
| PDGFRA_E125_F | NM_006206.3 | 61699224 | 5156 | PDGFRA | 0.303002 |
| PENK_E26_F | NM_006211.2 | 40254835 | 5179 | PENK | 0.437053 |
| PENK_P447_R | NM_006211.2 | 40254835 | 5179 | PENK | 0.445821 |
| PGR_P790_F | NM_000926.2 | 31981491 | 5241 | PGR | 0.31261 |
| PI3_E107_F | NM_002638.2 | 31657130 | 5266 | PI3 | 0.354377 |
| PITX2_E24_R | NM_000325.4 | 40316913 | 5308 | PITX2 | 0.351142 |
| PLXDC2_P914_R | NM_032812.7 | 40255004 | 84898 | PLXDC2 | 0.34481 |
| PTPRH_E173_F | NM_002842.2 | 67190343 | 5794 | PTPRH | 0.307824 |
| PTPRH_P255_F | NM_002842.2 | 67190343 | 5794 | PTPRH | 0.310071 |
| RAB32_P493_R | NM_006834.2 | 20127508 | 10981 | RAB32 | 0.337635 |
| RARA_P176_R | NM_000964.2 | 75812906 | 5914 | RARA | 0.333609 |
| RASGRF1_E16_F | NM_002891.3 | 24797098 | 5923 | RASGRF1 | 0.351307 |
| RBP1_P426_R | NM_002899.2 | 8400726 | 5947 | RBP1 | 0.314919 |
| RUNX1T1_P103_F | NM_175635.1 | 28329418 | 862 | RUNX1T1 | 0.35821 |
| S100A2_P1186_F | NM_005978.3 | 45269153 | 6273 | S100A2 | 0.304662 |
| SEMA3C_P642_F | NM_006379.2 | 32307182 | 10512 | SEMA3C | 0.342579 |
| SERPINB5_P19_R | NM_002639.2 | 52851464 | 5268 | SERPINB5 | 0.327176 |
| SEZ6L_P299_F | NM_021115.3 | 55956782 | 23544 | SEZ6L | 0.336576 |
| SLC5A8_E60_R | NM_145913.2 | 33942075 | 160728 | SLC5A8 | 0.340062 |
| SLC5A8_P38_R | NM_145913.2 | 33942075 | 160728 | SLC5A8 | 0.372619 |
| SLIT2_E111_R | NM_004787.1 | 4759145 | 9353 | SLIT2 | 0.302873 |
| SLIT2_P208_F | NM_004787.1 | 4759145 | 9353 | SLIT2 | 0.342402 |
| SOX17_P287_R | NM_022454.2 | 31077196 | 64321 | SOX17 | 0.337955 |
| SOX17_P303_F | NM_022454.2 | 31077196 | 64321 | SOX17 | 0.402562 |
| SOX1_P1018_R | NM_005986.2 | 30179899 | 6656 | SOX1 | 0.32451 |
| SOX1_P294_F | NM_005986.2 | 30179899 | 6656 | SOX1 | 0.473704 |
| ST6GAL1_P528_F | NM_173216.1 | 27765090 | 6480 | ST6GAL1 | 0.505492 |
| STAT5A_E42_F | NM_003152.2 | 21618341 | 6776 | STAT5A | 0.306205 |
| TAL1_P594_F | NM_003189.1 | 4507362 | 6886 | TAL1 | 0.362097 |
| TBX1_P885_R | NM_080646.1 | 18104949 | 6899 | TBX1 | 0.399846 |
| TERT_P360_R | NM_198255.1 | 38201701 | 7015 | TERT | 0.426258 |
| TFPI2_P9_F | NM_006528.2 | 31543803 | 7980 | TFPI2 | 0.392659 |
| THY1_P149_R | NM_006288.2 | 19923361 | 7070 | THY1 | 0.311299 |
| TNFRSF10D_E27_F | NM_003840.3 | 42544227 | 8793 | TNFRSF10D | 0.402129 |
| TPEF_seq_44_S88_R | NM_016192.2 | 12383050 | 23671 | TMEFF2 | 0.379537 |
| TRIM29_P261_F | NM_012101.2 | 17402908 | 23650 | TRIM29 | 0.380705 |
| TSP50_P137_F | NM_013270.2 | 31543829 | 29122 | TSP50 | 0.329338 |
| VAMP8_P114_F | NM_003761.2 | 14043025 | 8673 | VAMP8 | 0.303046 |
| WNT10B_P823_R | NM_003394.2 | 16936521 | 7480 | WNT10B | 0.334721 |
| WNT2_P217_F | NM_003391.1 | 4507926 | 7472 | WNT2 | 0.332473 |
| WT1_E32_F | NM_024424.2 | 65507816 | 7490 | WT1 | 0.43624 |
| WT1_P853_F | NM_024424.2 | 65507816 | 7490 | WT1 | 0.423456 |
| ZNF215_P129_R | NM_013250.1 | 7019582 | 7762 | ZNF215 | 0.310188 |
| ZNF215_P71_R | NM_013250.1 | 7019582 | 7762 | ZNF215 | 0.338592 |

TABLE 10
Differentially methylated probes in highly unstable 3q8pq20 tumors.

| | GenBank | | EntrezGene | | Adj. | Adj. | Adj. |
|---|---|---|---|---|---|---|---|
| Probe ID | Accession | GI | ID | Gene | p-value | p-value | p-value |
| NID1_P677_F | NM_002508.1 | 4505394 | 4811 | NID1 | 3.1111E−06 | 0.000552006 | 0.0260571 |
| AGXT_P180_F | NM_000030.1 | 4557288 | 189 | AGXT | 3.1111E−06 | 8.35732E−05 | 0.000359084 |
| NOS3_P38_F | NM_000603.3 | 48762674 | 4846 | NOS3 | 3.1111E−06 | 0.000552006 | 0.00377999 |
| SFN_E118_F | NM_006142.3 | 45238846 | 2810 | SFN | 4.8942E−06 | 0.00225537 | 0.241708 |
| SOX17_P303_F | NM_022454.2 | 31077196 | 64321 | SOX17 | 9.26124E−06 | 0.000501571 | 0.000164521 |
| SERPINB5_P19_R | NM_002639.2 | 52851464 | 5268 | SERPINB5 | 1.51362E−05 | 0.0103829 | 0.736282 |
| KRT5_E196_R | NM_000424.2 | 17318577 | 3852 | KRT5 | 1.51362E−05 | 0.00609735 | 0.536153 |
| DBC1_P351_R | NM_014618.1 | 7657008 | 1620 | DBC1 | 1.67364E−05 | 0.00181157 | 0.00264609 |
| TRIM29_E189_F | NM_012101.2 | 17402908 | 23650 | TRIM29 | 2.16481E−05 | 0.0162715 | 0.594008 |
| TRIM29_P261_F | NM_012101.2 | 17402908 | 23650 | TRIM29 | 4.28089E−05 | 0.0144537 | 0.499595 |
| AATK_E63_R | XM_927215.1 | 89041906 | 9625 | AATK | 6.02984E−05 | 0.0199569 | 0.647278 |
| CYP2E1_E53_R | NM_000773.3 | 75709190 | 1571 | CYP2E1 | 6.02984E−05 | 0.00587174 | 0.0411933 |
| RASGRF1_E16_F | NM_002891.3 | 24797098 | 5923 | RASGRF1 | 0.000156365 | 0.0925412 | 0.0127221 |
| SOX17_P287_R | NM_022454.2 | 31077196 | 64321 | SOX17 | 0.000185335 | 0.00268599 | 0.00210642 |
| LCN2_P86_R | NM_005564.2 | 38455401 | 3934 | LCN2 | 0.000203499 | 0.00227757 | 0.24433 |
| IL1RN_E42_F | NM_173843.1 | 27894320 | 3557 | IL1RN | 0.000280345 | 0.00989402 | 0.618086 |
| MDR1_seq_42_S300_R | NM_000927.3 | 42741658 | 5243 | ABCB1 | 0.000351442 | 0.0304916 | 0.112459 |
| FABP3_E113_F | NM_004102.3 | 62865867 | 2170 | FABP3 | 0.000439332 | 0.123549 | 0.591855 |
| NPY_E31_R | NM_000905.2 | 31542152 | 4852 | NPY | 0.00047077 | 0.0589057 | 0.0186261 |
| FGF1_P357_R | NM_033136.1 | 15055540 | 2246 | FGF1 | 0.000586337 | 0.035248 | 0.495658 |
| PTPRH_P255_F | NM_002842.2 | 67190343 | 5794 | PTPRH | 0.000722827 | 0.00626649 | 0.0259754 |
| AGTR1_P41_F | NM_000685.3 | 14043060 | 185 | AGTR1 | 0.000742962 | 0.0231095 | 0.0184185 |
| SLC5A8_E60_R | NM_145913.2 | 33942075 | 160728 | SLC5A8 | 0.00152201 | 0.0612212 | 0.100571 |
| GATA6_P726_F | NM_005257.3 | 40288196 | 2627 | GATA6 | 0.00171763 | 0.300971 | 0.344856 |
| PI3_E107_F | NM_002638.2 | 31657130 | 5266 | PI3 | 0.00172875 | 0.0110529 | 0.0220205 |
| DBC1_E204_F | NM_014618.1 | 7657008 | 1620 | DBC1 | 0.00200533 | 0.0296046 | 0.0575247 |
| OPCML_E219_R | NM_002545.3 | 59939898 | 4978 | OPCML | 0.00272792 | 0.0547983 | 0.140556 |
| ASCL1_E24_F | NM_004316.2 | 55743093 | 429 | ASCL1 | 0.00306726 | 0.0495832 | 0.0470806 |
| FGF3_E198_R | NM_005247.2 | 15451899 | 2248 | FGF3 | 0.00375183 | 0.183205 | 0.0748725 |
| IHH_E186_F | NM_002181.1 | 51467740 | 3549 | IHH | 0.0126057 | 0.110949 | 0.198983 |
| NTRK3_P636_R | NM_002530.2 | 59889559 | 4916 | NTRK3 | 0.0134184 | 0.0633468 | 0.0248722 |
| AGTR1_P154_F | NM_000685.3 | 14043060 | 185 | AGTR1 | 0.0166287 | 0.14858 | 0.120043 |
| MYH11_P22_F | NM_022844.1 | 13124874 | 4629 | MYH11 | 0.0175119 | 0.0463568 | 0.140556 |
| CHFR_P501_F | NM_018223.1 | 8922674 | 55743 | CHFR | 0.0193258 | 0.13386 | 0.0575247 |
| DLK1_E227_R | NM_003836.4 | 74136022 | 8788 | DLK1 | 0.021464 | 0.0532978 | 0.0417572 |
| NPY_P295_F | NM_000905.2 | 31542152 | 4852 | NPY | 0.021464 | 0.203065 | 0.0323715 |
| EPO_E244_R | NM_000799.2 | 62240996 | 2056 | EPO | 0.0343468 | 0.0579531 | 0.0654156 |
| ST6GAL1_P528_F | NM_173216.1 | 27765090 | 6480 | ST6GAL1 | 0.00412792 | 0.0268321 | 0.0293476 |
| PENK_P447_R | NM_006211.2 | 40254835 | 5179 | PENK | 0.035285 | 0.0737009 | 0.0126286 |
| FLT3_E326_R | NM_004119.1 | 4758395 | 2322 | FLT3 | 0.0183949 | 0.058043 | 0.00914553 |
| SEMA3C_P642_F | NM_006379.2 | 32307182 | 10512 | SEMA3C | 0.147317 | 0.0202112 | 0.000359084 |
| TERT_P360_R | NM_198255.1 | 38201701 | 7015 | TERT | 0.0216071 | 0.00976678 | 0.000241968 |
| EYA4_E277_F | NM_004100.2 | 26667248 | 2070 | EYA4 | 0.136266 | 0.012748 | 0.00264609 |
| SOX1_P294_F | NM_005986.2 | 30179899 | 6656 | SOX1 | 0.0576915 | 0.0268321 | 0.0172274 |
| HOM11_P698_F | NM_005523.4 | 24497552 | 3207 | HOXA11 | 0.00319023 | 0.000176805 | 0.000158227 |
| ADCYAP1_P398_F | NM_001117.2 | 10947062 | 116 | ADCYAP1 | 0.00728019 | 0.00314866 | 0.00129486 |
| HOXA9_P1141_R | NM_002142.3 | 24497558 | 3205 | HOXA9 | 0.000102542 | 4.37632E−05 | 8.79557E−06 |
| PENK_E26_F | NM_006211.2 | 40254835 | 5179 | PENK | 4.39464E−05 | 0.000086375 | 9.43297E−06 |
| HS3ST2_P171_F | NM_006043.1 | 5174462 | 9956 | HS3ST2 | 7.60996E−06 | 0.000086375 | 3.09903E−05 |
| HOXA9_E252_R | NM_002142.3 | 24497558 | 3205 | HOXA9 | 0.000809034 | 5.24455E−05 | 3.17908E−05 |
| MOS_E60_R | NM_005372.1 | 4885488 | 4342 | MOS | 0.000439332 | 0.00016613 | 0.000158227 |
| ADCYAP1_P455_R | NM_001117.2 | 10947062 | 116 | ADCYAP1 | 4.28089E−05 | 0.000290683 | 0.000158227 |
| HS3ST2_E145_R | NM_006043.1 | 5174462 | 9956 | HS3ST2 | 5.47191E−05 | 0.00181157 | 0.000349679 |
| MT1A_E13_R | NM_005946.2 | 71274112 | 4489 | MT1A | 0.00248209 | 0.00016613 | 0.000441245 |
| MT1A_P49_R | NM_005946.2 | 71274112 | 4489 | MT1A | 0.004164 | 0.000646403 | 0.00101078 |
| HTR1B_P222_F | NM_000863.1 | 4504532 | 3351 | HTR1B | 0.013391 | 0.00855737 | 0.00129486 |

| Probe ID | methylation difference | methylation difference | methylation difference | Significant | Significant | Significant |
|---|---|---|---|---|---|---|
| NID1_P677_F | −0.705923 | 0.409399 | 0.225215 | Significant | NS | NS |
| AGXT_P180_F | −0.666864 | 0.48076 | 0.366161 | Significant | NS | NS |
| NOS3_P38_F | −0.574772 | 0.343743 | 0.256117 | Significant | NS | NS |
| SFN_E118_F | −0.582424 | 0.31039 | 0.107414 | Significant | NS | NS |
| SOX17_P303_F | 0.717937 | −0.479966 | −0.490993 | Significant | NS | NS |
| SERPINB5_P19_R | −0.651656 | 0.309721 | 0.043595 | Significant | NS | NS |
| KRT5_E196_R | −0.578841 | 0.292749 | 0.066543 | Significant | NS | NS |
| DBC1_P351_R | 0.702001 | −0.43418 | −0.380495 | Significant | NS | NS |
| TRIM29_E189_F | −0.58502 | 0.268438 | 0.061692 | Significant | NS | NS |
| TRIM29_P261_F | −0.736165 | 0.36866 | 0.103282 | Significant | NS | NS |
| AATK_E63_R | −0.561057 | 0.274708 | 0.056587 | Significant | NS | NS |
| CYP2E1_E53_R | −0.527176 | 0.317953 | 0.212281 | Significant | NS | NS |
| RASGRF1_E16_F | 0.596039 | −0.226412 | −0.327785 | Significant | NS | NS |
| SOX17_P287_R | 0.550374 | −0.405948 | −0.389854 | Significant | NS | NS |
| LCN2_P86_R | −0.501623 | 0.381967 | 0.132093 | Significant | NS | NS |
| IL1RN_E42_F | −0.531977 | 0.335781 | 0.066574 | Significant | NS | NS |
| MDR1_seq_42_S300_R | 0.811387 | −0.429234 | −0.298149 | Significant | NS | NS |
| FABP3_E113_F | 0.570488 | −0.222 | −0.081074 | Significant | NS | NS |
| NPY_E31_R | 0.620029 | −0.301035 | −0.357479 | Significant | NS | NS |
| FGF1_P357_R | −0.510099 | 0.281343 | 0.093665 | Significant | NS | NS |
| PTPRH_P255_F | −0.500194 | 0.37455 | 0.280274 | Significant | NS | NS |
| AGTR1_P41_F | 0.777788 | −0.473484 | −0.473675 | Significant | NS | NS |
| SLC5A8_E60_R | 0.551788 | −0.296029 | −0.244196 | Significant | NS | NS |
| GATA6_P726_F | 0.578697 | −0.182632 | −0.155315 | Significant | NS | NS |
| PI3_E107_F | −0.539683 | 0.407957 | 0.342551 | Significant | NS | NS |
| DBC1_E204_F | 0.60107 | −0.388879 | −0.318285 | Significant | NS | NS |
| OPCML_E219_R | 0.609614 | −0.365568 | −0.261118 | Significant | NS | NS |
| ASCL1_E24_F | 0.54168 | −0.336403 | −0.314723 | Significant | NS | NS |
| FGF3_E198_R | 0.544293 | −0.239586 | −0.288964 | Significant | NS | NS |
| IHH_E186_F | 0.524807 | −0.314703 | −0.241612 | Significant | NS | NS |
| NTRK3_P636_R | 0.52026 | −0.366155 | −0.420576 | Significant | NS | NS |
| AGTR1_P154_F | 0.516303 | −0.296998 | −0.295823 | Significant | NS | NS |
| MYH11_P22_F | 0.53147 | −0.424168 | −0.292759 | Significant | NS | NS |
| CHFR_P501_F | 0.586746 | −0.358052 | −0.422083 | Significant | NS | NS |
| DLK1_E227_R | 0.53773 | −0.435898 | −0.423288 | Significant | NS | NS |
| NPY_P295_F | 0.580127 | −0.316679 | −0.480994 | Significant | NS | NS |
| EPO_E244_R | 0.59881 | −0.519064 | −0.466962 | Significant | NS | NS |
| ST6GAL1_P528_F | 0.731643 | −0.523382 | −0.483266 | Significant | Significant | NS |
| PENK_P447_R | 0.477074 | −0.386456 | −0.527033 | NS | NS | Significant |
| FLT3_E326_R | 0.512158 | −0.391134 | −0.517238 | Significant | NS | Significant |
| SEMA3C_P642_F | −0.214843 | 0.332468 | 0.519865 | NS | NS | Significant |
| TERT_P360_R | 0.408172 | −0.447039 | −0.641584 | NS | NS | Significant |
| EYA4_E277_F | 0.328514 | −0.538495 | −0.627866 | NS | Significant | Significant |
| SOX1_P294_F | 0.463084 | −0.516989 | −0.539242 | NS | Significant | Significant |
| HOM11_P698_F | 0.439617 | −0.586552 | −0.554535 | NS | Significant | Significant |
| ADCYAP1_P398_F | 0.466225 | −0.502248 | −0.524235 | NS | Significant | Significant |
| HOXA9_P1141_R | 0.620713 | −0.713578 | −0.741006 | Significant | Significant | Significant |
| PENK_E26_F | 0.630238 | −0.589339 | −0.652701 | Significant | Significant | Significant |
| HS3ST2_P171_F | 0.700822 | −0.541805 | −0.547244 | Significant | Significant | Significant |
| HOXA9_E252_R | 0.503226 | −0.67755 | −0.615136 | Significant | Significant | Significant |
| MOS_E60_R | 0.630517 | −0.690014 | −0.636402 | Significant | Significant | Significant |
| ADCYAP1_P455_R | 0.644028 | −0.530203 | −0.526402 | Significant | Significant | Significant |
| HS3ST2_E145_R | 0.718585 | −0.500912 | −0.548149 | Significant | Significant | Significant |
| MT1A_E13_R | 0.511971 | −0.67013 | −0.548137 | Significant | Significant | Significant |
| MT1A_P49_R | 0.567717 | −0.688316 | −0.606695 | Significant | Significant | Significant |
| HTR1B_P222_F | 0.573875 | −0.584619 | −0.701384 | Significant | Significant | Significant |

TABLE 11
Enrichment of Gene Ontology processes represented by the significantly differentially methylated probes in highly unstable 3q8pq20 tumors

| Gene Ontology | | Entrez Gene | Visible Base | Visible |
|---|---|---|---|---|
| Process | Canonical Name | Neighbors | Neighbors | Enrichment |
| GO: 0048645 | organ formation | 16 | 4 | 0.003231132 |
| GO: 0085029 | extracellular matrix assembly | 3 | 2 | 0.00495841 |
| GO: 0003044 | regulation of systemic arterial blood pressure mediated by a chemical signal | 3 | 2 | 0.00495841 |
| GO: 0001990 | regulation of systemic arterial blood pressure by hormone | 3 | 2 | 0.00495841 |
| GO: 0003151 | outflow tract morphogenesis | 3 | 2 | 0.00495841 |
| GO: 0060479 | lung cell differentiation | 4 | 2 | 0.009657499 |
| GO: 0060487 | lung epithelial cell differentiation | 4 | 2 | 0.009657499 |
| GO: 0030855 | epithelial cell differentiation | 36 | 5 | 0.013844549 |
| GO: 0042312 | regulation of vasodilation | 5 | 2 | 0.015675955 |
| GO: 0055093 | response to hyperoxia | 5 | 2 | 0.015675955 |
| GO: 0006814 | sodium ion transport | 5 | 2 | 0.015675955 |
| GO: 0003073 | regulation of systemic arterial blood pressure | 5 | 2 | 0.015675955 |
| GO: 0050886 | endocrine process | 5 | 2 | 0.015675955 |
| GO: 0030198 | extracellular matrix organization | 25 | 4 | 0.017184145 |
| GO: 0045165 | cell fate commitment | 39 | 5 | 0.01931485 |
| GO: 0051094 | positive regulation of developmental process | 103 | 9 | 0.019675782 |
| GO: 0008217 | regulation of blood pressure | 15 | 3 | 0.021492082 |
| GO: 0030001 | metal ion transport | 27 | 4 | 0.02246693 |
| GO: 0042311 | vasodilation | 6 | 2 | 0.022902063 |
| GO: 0001101 | response to acid | 6 | 2 | 0.022902063 |
| GO: 0044106 | cellular amine metabolic process | 16 | 3 | 0.025706147 |
| GO: 0009719 | response to endogenous stimulus | 110 | 9 | 0.029485867 |
| GO: 0035051 | cardiac cell differentiation | 7 | 2 | 0.031230652 |
| GO: 0006812 | cation transport | 30 | 4 | 0.032098851 |
| GO: 0060428 | lung epithelium development | 8 | 2 | 0.040562773 |
| GO: 0048678 | response to axon injury | 8 | 2 | 0.040562773 |
| GO: 0045666 | positive regulation of neuron differentiation | 8 | 2 | 0.040562773 |
| GO: 0035295 | tube development | 80 | 7 | 0.040621923 |
| GO: 0048073 | regulation of eye pigmentation | 1 | 1 | 0.041830065 |
| GO: 0048069 | eye pigmentation | 1 | 1 | 0.041830065 |
| GO: 0048086 | negative regulation of developmental pigmentation | 1 | 1 | 0.041830065 |
| GO: 0003148 | outflow tract septum morphogenesis | 1 | 1 | 0.041830065 |
| GO: 0009070 | serine family amino acid biosynthetic process | 1 | 1 | 0.041830065 |
| GO: 0008343 | adult feeding behavior | 1 | 1 | 0.041830065 |
| GO: 0032107 | regulation of response to nutrient levels | 1 | 1 | 0.041830065 |
| GO: 0032104 | regulation of response to extracellular stimulus | 1 | 1 | 0.041830065 |
| GO: 0032095 | regulation of response to food | 1 | 1 | 0.041830065 |
| GO: 0060411 | cardiac septum morphogenesis | 1 | 1 | 0.041830065 |
| GO: 0032109 | positive regulation of response to nutrient levels | 1 | 1 | 0.041830065 |
| GO: 0032106 | positive regulation of response to extracellular stimulus | 1 | 1 | 0.041830065 |
| GO: 0006625 | protein targeting to peroxisome | 1 | 1 | 0.041830065 |
| GO: 0010288 | response to lead ion | 1 | 1 | 0.041830065 |
| GO: 0015891 | siderophore transport | 1 | 1 | 0.041830065 |
| GO: 0033214 | iron assimilation by chelation and transport | 1 | 1 | 0.041830065 |
| GO: 0015688 | iron chelate transport | 1 | 1 | 0.041830065 |
| GO: 0033212 | iron assimilation | 1 | 1 | 0.041830065 |
| GO: 0015892 | siderophore-iron transport | 1 | 1 | 0.041830065 |
| GO: 0043574 | peroxisomal transport | 1 | 1 | 0.041830065 |
| GO: 0032098 | regulation of appetite | 1 | 1 | 0.041830065 |
| GO: 0070572 | positive regulation of neuron projection regeneration | 1 | 1 | 0.041830065 |
| GO: 0048680 | positive regulation of axon regeneration | 1 | 1 | 0.041830065 |
| GO: 0006835 | dicarboxylic acid transport | 1 | 1 | 0.041830065 |
| GO: 0048677 | axon extension involved in regeneration | 1 | 1 | 0.041830065 |
| GO: 0048682 | sprouting of injured axon | 1 | 1 | 0.041830065 |
| GO: 0018298 | protein-chromophore linkage | 1 | 1 | 0.041830065 |
| GO: 0060164 | regulation of timing of neuron differentiation | 1 | 1 | 0.041830065 |
| GO: 0042866 | pyruvate biosynthetic process | 1 | 1 | 0.041830065 |
| GO: 0043249 | erythrocyte maturation | 1 | 1 | 0.041830065 |
| GO: 0021527 | spinal cord association neuron differentiation | 1 | 1 | 0.041830065 |
| GO: 0031033 | myosin filament assembly or disassembly | 1 | 1 | 0.041830065 |
| GO: 0006081 | cellular aldehyde metabolic process | 1 | 1 | 0.041830065 |
| GO: 0046487 | glyoxylate metabolic process | 1 | 1 | 0.041830065 |
| GO: 0071214 | cellular response to abiotic stimulus | 1 | 1 | 0.041830065 |
| GO: 0060220 | camera-type eye photoreceptor cell fate commitment | 1 | 1 | 0.041830065 |
| GO: 0048074 | negative regulation of eye pigmentation | 1 | 1 | 0.041830065 |
| GO: 0042706 | eye photoreceptor cell fate commitment | 1 | 1 | 0.041830065 |
| GO: 0046552 | photoreceptor cell fate commitment | 1 | 1 | 0.041830065 |
| GO: 0003406 | retinal pigment epithelium development | 1 | 1 | 0.041830065 |
| GO: 0030704 | vitelline membrane formation | 1 | 1 | 0.041830065 |
| GO: 0071371 | cellular response to gonadotropin stimulus | 1 | 1 | 0.041830065 |
| GO: 0007031 | peroxisome organization | 1 | 1 | 0.041830065 |
| GO: 0003081 | regulation of systemic arterial blood pressure by renin-angiotensin | 1 | 1 | 0.041830065 |
| GO: 0003071 | renal system process involved in regulation of systemic arterial blood pressure | 1 | 1 | 0.041830065 |
| GO: 0060913 | cardiac cell fate determination | 1 | 1 | 0.041830065 |
| GO: 0033864 | positive regulation of NAD(P)H oxidase activity | 1 | 1 | 0.041830065 |
| GO: 0003072 | renal control of peripheral vascular resistance involved in regulation of systemic arterial | 1 | 1 | 0.041830065 |
| | blood pressure | | | |
| GO: 0044062 | regulation of excretion | 1 | 1 | 0.041830065 |
| GO: 0003078 | regulation of natriuresis | 1 | 1 | 0.041830065 |
| GO: 0002034 | regulation of blood vessel size by renin-angiotensin | 1 | 1 | 0.041830065 |
| GO: 0002018 | renin-angiotensin regulation of aldosterone production | 1 | 1 | 0.041830065 |
| GO: 0021516 | dorsal spinal cord development | 1 | 1 | 0.041830065 |
| GO: 0060911 | cardiac cell fate commitment | 1 | 1 | 0.041830065 |
| GO: 0060956 | endocardial cell differentiation | 1 | 1 | 0.041830065 |
| GO: 0003348 | cardiac endothelial cell differentiation | 1 | 1 | 0.041830065 |
| GO: 0060214 | endocardium formation | 1 | 1 | 0.041830065 |
| GO: 0030823 | regulation of cGMP metabolic process | 1 | 1 | 0.041830065 |
| GO: 0030826 | regulation of cGMP biosynthetic process | 1 | 1 | 0.041830065 |
| GO: 0021895 | cerebral cortex neuron differentiation | 1 | 1 | 0.041830065 |
| GO: 0014016 | neuroblast differentiation | 1 | 1 | 0.041830065 |
| GO: 0014017 | neuroblast fate commitment | 1 | 1 | 0.041830065 |
| GO: 0003357 | noradrenergic neuron differentiation | 1 | 1 | 0.041830065 |
| GO: 0019265 | glycine biosynthetic process, by transamination of glyoxylate | 1 | 1 | 0.041830065 |
| GO: 0046724 | oxalic acid secretion | 1 | 1 | 0.041830065 |
| GO: 0006544 | glycine metabolic process | 1 | 1 | 0.041830065 |
| GO: 0006545 | glycine biosynthetic process | 1 | 1 | 0.041830065 |
| GO: 0002016 | regulation of blood volume by renin-angiotensin | 1 | 1 | 0.041830065 |
| GO: 0060430 | lung saccule development | 1 | 1 | 0.041830065 |
| GO: 0014745 | negative regulation of muscle adaptation | 1 | 1 | 0.041830065 |
| GO: 0014740 | negative regulation of muscle hyperplasia | 1 | 1 | 0.041830065 |
| GO: 0014900 | muscle hyperplasia | 1 | 1 | 0.041830065 |
| GO: 0014738 | regulation of muscle hyperplasia | 1 | 1 | 0.041830065 |
| GO: 0031284 | positive regulation of guanylate cyclase activity | 1 | 1 | 0.041830065 |
| GO: 0014806 | smooth muscle hyperplasia | 1 | 1 | 0.041830065 |
| GO: 0031282 | regulation of guanylate cyclase activity | 1 | 1 | 0.041830065 |
| GO: 0003310 | pancreatic A cell differentiation | 1 | 1 | 0.041830065 |
| GO: 0003309 | pancreatic B cell differentiation | 1 | 1 | 0.041830065 |
| GO: 0007616 | long-term memory | 1 | 1 | 0.041830065 |
| GO: 0008652 | cellular amino acid biosynthetic process | 1 | 1 | 0.041830065 |
| GO: 0016098 | monoterpenoid metabolic process | 1 | 1 | 0.041830065 |
| GO: 0032100 | positive regulation of appetite | 1 | 1 | 0.041830065 |
| GO: 0032097 | positive regulation of response to food | 1 | 1 | 0.041830065 |
| GO: 0008218 | bioluminescence | 1 | 1 | 0.041830065 |
| GO: 0090136 | epithelial cell-cell adhesion | 1 | 1 | 0.041830065 |
| GO: 0021778 | oligodendrocyte cell fate specification | 1 | 1 | 0.041830065 |
| GO: 0021530 | spinal cord oligodendrocyte cell fate specification | 1 | 1 | 0.041830065 |
| GO: 0021779 | oligodendrocyte cell fate commitment | 1 | 1 | 0.041830065 |
| GO: 0021780 | glial cell fate specification | 1 | 1 | 0.041830065 |
| GO: 0021529 | spinal cord oligodendrocyte cell differentiation | 1 | 1 | 0.041830065 |
| GO: 0071000 | response to magnetism | 1 | 1 | 0.041830065 |
| GO: 0071688 | striated muscle myosin thick filament assembly | 1 | 1 | 0.041830065 |
| GO: 0071259 | cellular response to magnetism | 1 | 1 | 0.041830065 |
| GO: 0030241 | skeletal muscle myosin thick filament assembly | 1 | 1 | 0.041830065 |
| GO: 0060163 | subpallium neuron fate commitment | 1 | 1 | 0.041830065 |
| GO: 0048739 | cardiac muscle fiber development | 1 | 1 | 0.041830065 |
| GO: 0021892 | cerebral cortex GABAergic interneuron differentiation | 1 | 1 | 0.041830065 |
| GO: 0007400 | neuroblast fate determination | 1 | 1 | 0.041830065 |
| GO: 0060166 | olfactory pit development | 1 | 1 | 0.041830065 |
| GO: 0003359 | noradrenergic neuron fate commitment | 1 | 1 | 0.041830065 |
| GO: 0070849 | response to epidermal growth factor stimulus | 1 | 1 | 0.041830065 |
| GO: 0060165 | regulation of timing of subpallium neuron differentiation | 1 | 1 | 0.041830065 |
| GO: 0031034 | myosin filament assembly | 1 | 1 | 0.041830065 |
| GO: 0060486 | Clara cell differentiation | 1 | 1 | 0.041830065 |
| GO: 0014866 | skeletal myofibril assembly | 1 | 1 | 0.041830065 |
| GO: 0048690 | regulation of axon extension involved in regeneration | 1 | 1 | 0.041830065 |
| GO: 0048686 | regulation of sprouting of injured axon | 1 | 1 | 0.041830065 |
| GO: 0048687 | positive regulation of sprouting of injured axon | 1 | 1 | 0.041830065 |
| GO: 0048691 | positive regulation of axon extension involved in regeneration | 1 | 1 | 0.041830065 |
| GO: 0009069 | serine family amino acid metabolic process | 1 | 1 | 0.041830065 |
| GO: 0043062 | extracellular structure organization | 33 | 4 | 0.043858382 |
| GO: 0048545 | response to steroid hormone stimulus | 66 | 6 | 0.049379844 |

### Gene Amplification Occurs in Dysplasia

In addition to the low level gains and losses discussed above, we observed that dysplasia genomes harbored amplifications, defined as focal regions of higher level increased copy number. Previously, we reported that oral SCC characteristically amplify narrow regions of the genome (<3 Mb) and identified 18 such recurrent amplicons (Snijders et al. 2005). In the 29 dysplasia cases with no known association with cancer, we found two of these amplicons at 11q13 (CCND1, PAK1) and 20p12.2 (JAG1) to be present, as well as amplification at 2q11.2 in two dysplasia cases and two non-recurrent amplicons at 20q13.33 and 21q21.3 (FIG. 1B and Table 12). The amplification at 21q21.3, however, spans a region that is gained in ≧15% of SCC cases (Table 6) and a likely driver gene for this amplicon is MIR155. Although the 2q11.2 amplicon had not been observed previously in the 89 oral SCCs (Snijders et al. 2005), we had reported it in an oral SCC cell line (Hermsen et al. 2005) and it has recently been reported by others in dysplasia (Garnis et al. 2009). The recurrent amplicons are present in both 3q8pq20 and non-3q8pq20 dysplasia and SCC genomes (FIGS. 1 and 4), and thus their formation appears to be mediated by processes independent of those driving low level gains and losses.

TABLE 12
Amplicons in 29 dysplasia samples from patients with no known history of oral cancer

| Dysplasia | | | SCC^{a} | Proximal | | Distal | | Candidate |
|---|---|---|---|---|---|---|---|---|
| case no. | Cyto-Band | Size (Mb) | (%) | flanking clone | STS | flanking clone | STS | oncogenes |
| 5779, 5914 | 2q11.2 | 3.7 | 0%^{b} | RP11-327M19 | | RP11-629A22 | AFMB355ZG1 | CIAO1, CNNM3 |
| 5952 | 11q13.3 | 1.6 | 11% | CTD-2080I19 | RH7839 | RP11-120P20 | SHGC-4518 | CCND1, EMS1 |
| 5952 | 11q13.5 | 0.9 | 2% | CTC-352E23 | RH52308 | RP11-98G24 | SHGC-31540 | PAK1 |
| 6390 | 20p12.2 | 1.2 | 3% | RMC20P160 | WI-7829 | RMC20P178 | D20S186 | JAG1 |
| 6390 | 20q13.33 | 3.2 | 0% | RP11-94A18 | AFM218XE7 | RP11-358D14 | X70940 | CDH4, PSMA7 |
| 5779 | 21q21.3 | 4.8 | 0%^{c} | RP11-86J21 | AFMA081WF1 | RP11-115H17 | SHGC-11277 | ADRM1, LAMA5, NTSR1, BIRC7 |
| | | | | | | | | MIR155 |

^{a}Frequency reported in oral SCC cohort#1 by Snijders et al. (Snijders et al. 2005)
^{b}Although the 2q11.2 amplicon had not been observed previously in SCC cohort#1 as a recurrent amplicon (Snijders et al. 2005), we had reported it in an oral SCC cell line (Hermsen et al. 2005) and it has recently been reported by others in dysplasia (Garnis et al. 2009).
^{c}The region is gained in ≧15% of SCC cases.

### Oral Cancer Subtypes Differ in Clinical Behavior

Considered together the distribution of copy number aberrations in dysplasia and SCC suggest that there are two distinct routes to oral cancer, one associated with greater genome instability and acquisition of +3q, −8p, +8q and/or +20 in pre-malignant stages and the other lacking chromosomal level instability detectable by CGH. Potential differences in developmental pathways leading to oral cancer are likely to impact clinical behavior. Indeed, we observed a highly significant association of 3q8pq20 status with pathologic cervical (neck) lymph node status (odds ratio 11.5 (CI 1.5, 521.8); Fisher's exact test p=0.006), i.e. neck metastasis (N+) was present in 46% (22/48) of 3q8pq20 tumors and in only 7% (1 of 15) of non-3q8pq20 tumors (Table 13 and Table 14).

TABLE 13
Biomarker prediction of pathological cervical node status
in two independent oral SCC cohorts.

| | Cohort#2 (n = 63) | | VUMC (n = 16) | |
|---|---|---|---|---|
| Nodal status | N0 | N+ | N0 | N+ |
| 3q8pq20 | 26 | 22 | 3 | 10 |
| non-3q8pq20 | 14 | 1 | 3 | 0 |

| | | |
|---|---|---|
| Sensitivity | 0.96 | 1.00 |
| Specificity | 0.35 | 0.50 |
| Positive predictive value | 0.46 | 0.77 |
| Negative predictive value | 0.93 | 1.00 |
| p-value | 0.0058 | 0.036 |
| Sample Odds Ratio | 11.85 | |
| | (CI 1.52, 521.82) | |

TABLE 14
Patient and tumor characteristics relative to tumor subtype

| | | | | | | 95% confidence | |
|---|---|---|---|---|---|---|---|
| | | | non- | p- | Odds | interval | |
| | n | 3q8pq20 | 3q8pq20 | value | Ratio | lower | upper |
| Nodal status | 63 | 48 | 15 | 0.006 | 11.494 | 1.516 | 521.823 |
| N0 | | 26 | 14 | | | | |
| N+ | | 22 | 1 | | | | |
| Age | 63 | | | 0.018 | 0.199 | 0.032 | 0.870 |
| <65 | | 27 | 3 | | | | |
| ≧65 | | 21 | 12 | | | | |
| Gender | 63 | | | 0.765 | 1.329 | 0.347 | 5.010 |
| Female | | 19 | 7 | | | | |
| Male | | 29 | 8 | | | | |
| Tumor size | 62 | | | 1.000 | 0.994 | 0.260 | 3.729 |
| <2.7 cm | | 22 | 7 | | | | |
| ≧2.7 cm | | 25 | 8 | | | | |
| Tumor thickness | 50 | | | 0.314 | 2.228 | 0.473 | 12.171 |
| <1.3 cm | | 17 | 7 | | | | |
| ≧1.3 cm | | 22 | 4 | | | | |
| Tobacco use | 46 | | | 0.355 | 2.443 | 0.302 | 17.653 |
| never | | 9 | 3 | | | | |
| ever | | 30 | 4 | | | | |
| Tobacco use excluding | 45 | | | 0.362 | 2.363 | 0.291 | 17.099 |
| snuff | | | | | | | |
| never | | 9 | 3 | | | | |
| ever | | 29 | 4 | | | | |
| Tobacco use | 46 | | | 0.250 | NA | NA | NA |
| never | | 9 | 3 | | | | |
| previous | | 11 | 3 | | | | |
| current | | 19 | 1 | | | | |
| Tobacco use excluding | 45 | | | 0.286 | NA | NA | NA |
| snuff | | | | | | | |
| never | | 9 | 3 | | | | |
| previous | | 11 | 3 | | | | |
| current | | 18 | 1 | | | | |
| Alcohol use | 43 | | | 0.347 | 2.558 | 0.310 | 18.918 |
| never | | 8 | 3 | | | | |
| ever | | 28 | 4 | | | | |
| Alcohol use | 43 | | | 0.376 | NA | NA | NA |
| never | | 8 | 3 | | | | |
| previous | | 7 | 0 | | | | |
| current | | 21 | 4 | | | | |
| Site | 57 | | | 0.117 | NA | NA | NA |
| buccal mucosa | | 7 | 2 | | | | |
| floor of mouth | | 10 | 1 | | | | |
| gingiva | | 5 | 6 | | | | |
| retromolar region | | 5 | 0 | | | | |
| tongue | | 15 | 6 | | | | |

The presence of metastases to the cervical lymph nodes is the major determinant of survival for oral SCC patients (O'Brien et al. 1986; Whitehurst et al. 1977). The differential risk for metastasis in the 3q8pq20 and non-3q8pq20 oral SCC subtypes indicates that chromosomal aberrations +3q, −8p, +8q and +20 provide a potential biomarker to identify patients with no or low risk of metastasis. To confirm this observation, we investigated the association of nodal status and 3q8pq20 status in the independent cohort of oral SCC patients from the Netherlands (Smeets et al. 2009) for which copy number and pathologic node status were available (VUMC, Table 13). In this cohort, we also found the non-3q8pq20 subtype to be at low risk for metastasis (Fisher's exact test p=0.036). We note in particular that the sensitivity and negative predictive value for metastasis (i.e. ability to predict N0 cases at the time of biopsy) were 96% and 93%, respectively in SCC cohort#2 and both were 100% in the Dutch cohort (Table 13). We also observed a modest association with age in cohort#2, non-3q8pq20 tumors were more frequent in patients older than 65 years (p=0.018, Table 14), but not in the Dutch cohort.

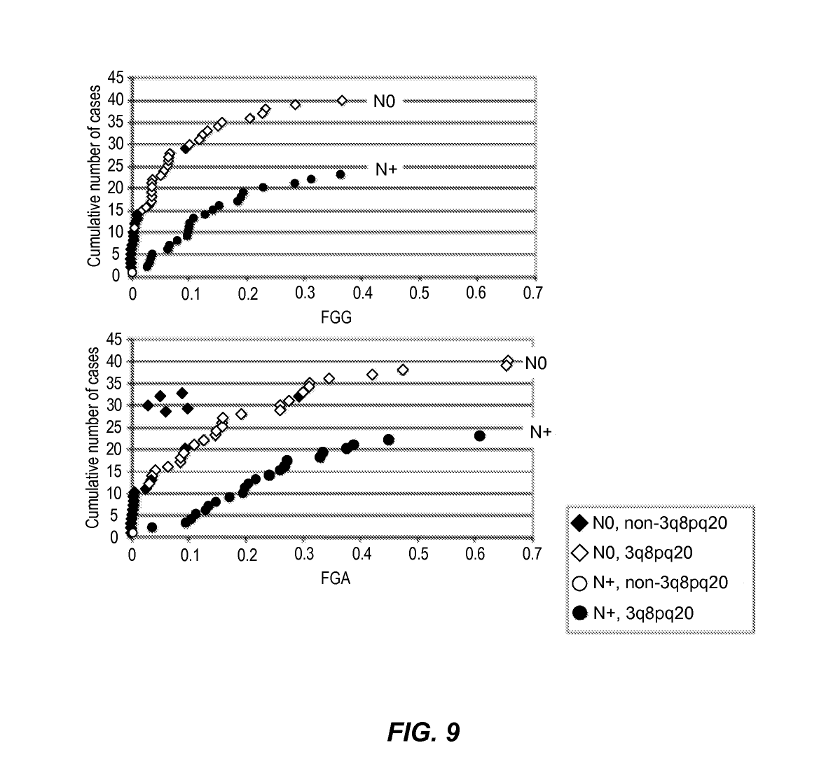

Since the 3q8pq20 and non-3q8pq20 subtypes also differ in genomic instability, we considered association of genome instability measures with clinical characteristics in cohort#2. On the one hand, although genome instability is commonly reported to be correlated with measures of poor prognosis, we found no association of any genome instability measures with recurrence free survival, disease free survival or overall survival in cohort#2 (log rank test, data not shown). On the other hand, we observed significant association of nodal status with increased numbers of whole chromosome copy number changes (p=0.046), fraction of the genome gained (FGG, p=0.004) and fraction of the genome altered (FGA, p=0.024), suggesting that these measures may also serve as biomarkers of nodal status (Table 15). We did not, however, find a clear cutpoint for prediction of nodal status by either measure (FIG. 9). Nevertheless, by applying maximally selected chi-square statistics (Miller and Siegmund 1982), we obtained cutpoints at 0.065 and 0.095 for FGG and FGA, respectively, yielding sensitivity, specificity, positive predictive value and negative predictive value of 74%, 68%, 57% and 82% for FGG and 91%, 48%, 50% and 90% for FGA compared to 96%, 35%, 46% and 93% for 3q8pq20 status (Table 13). Thus, with these cutpoints, FGG and FGA both correctly identify more of the true N0 cases; however, more N+ cases are mistakenly called N0.

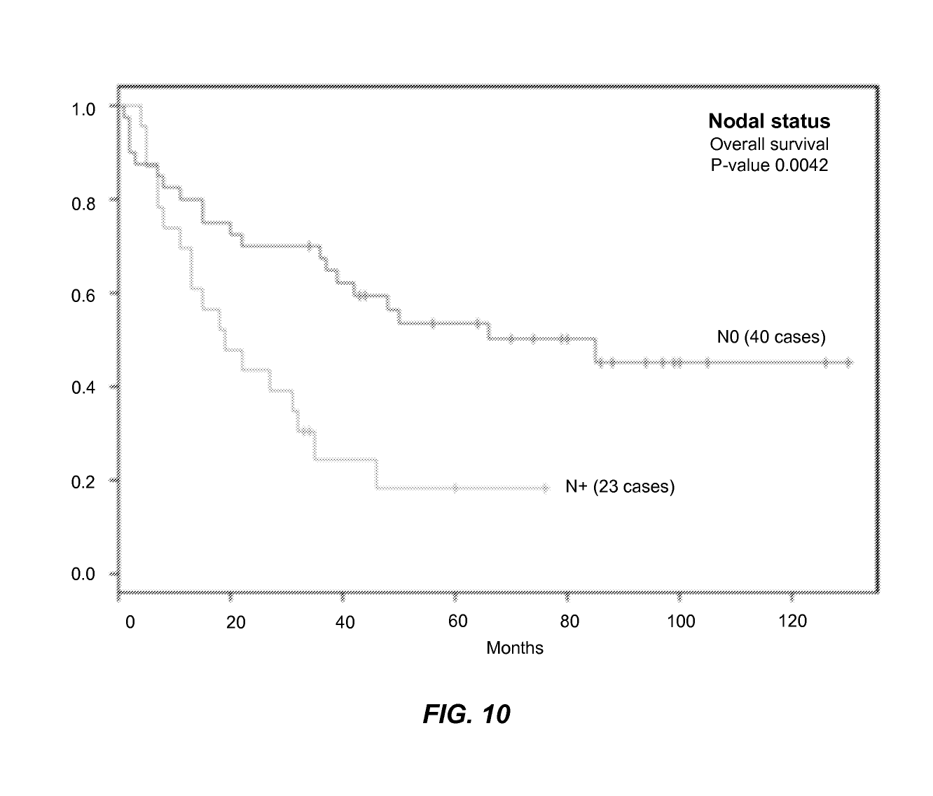

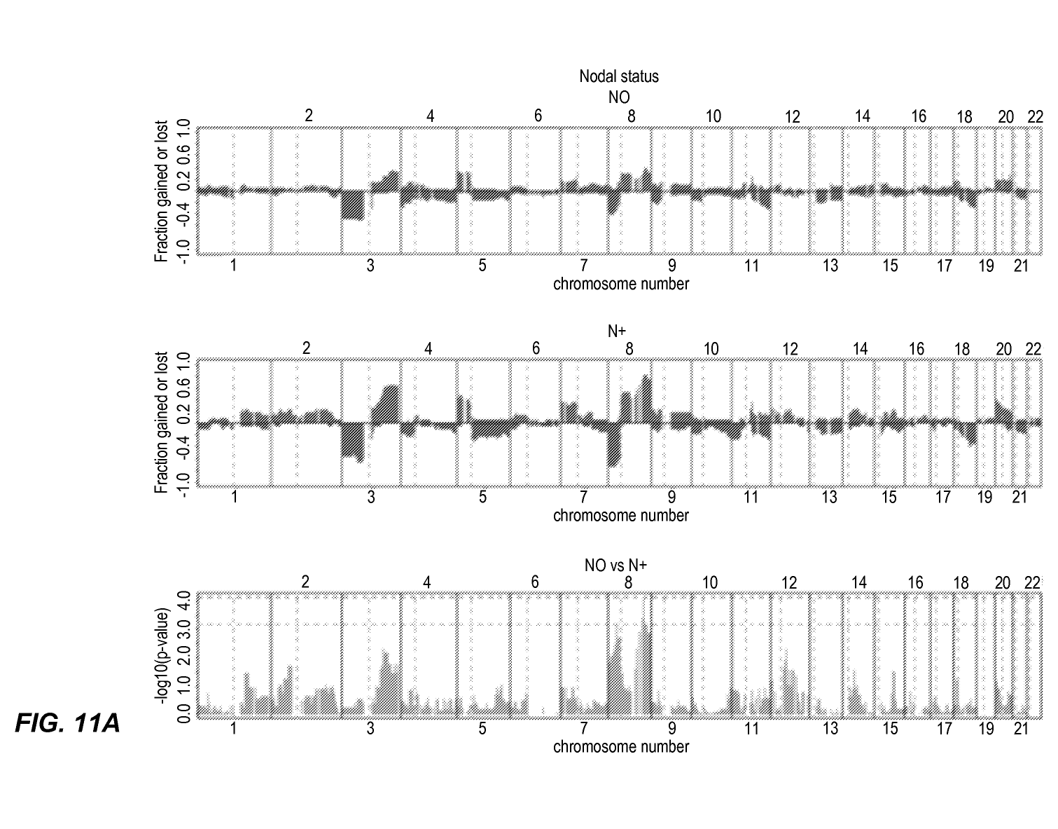

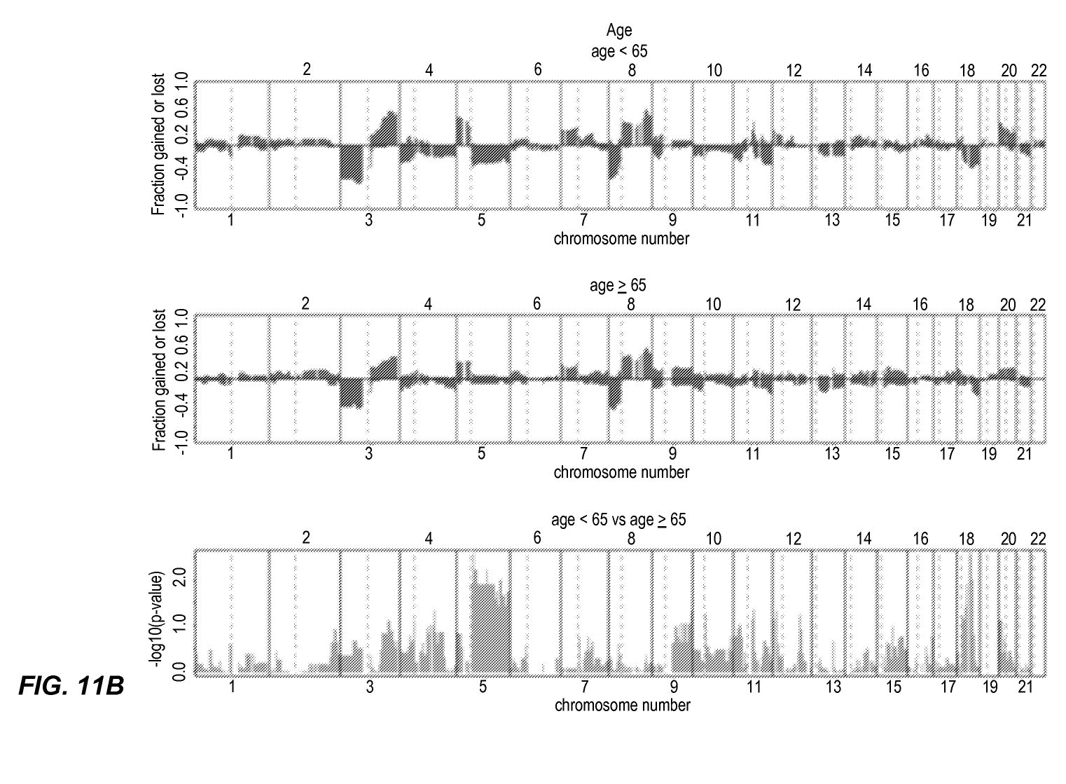

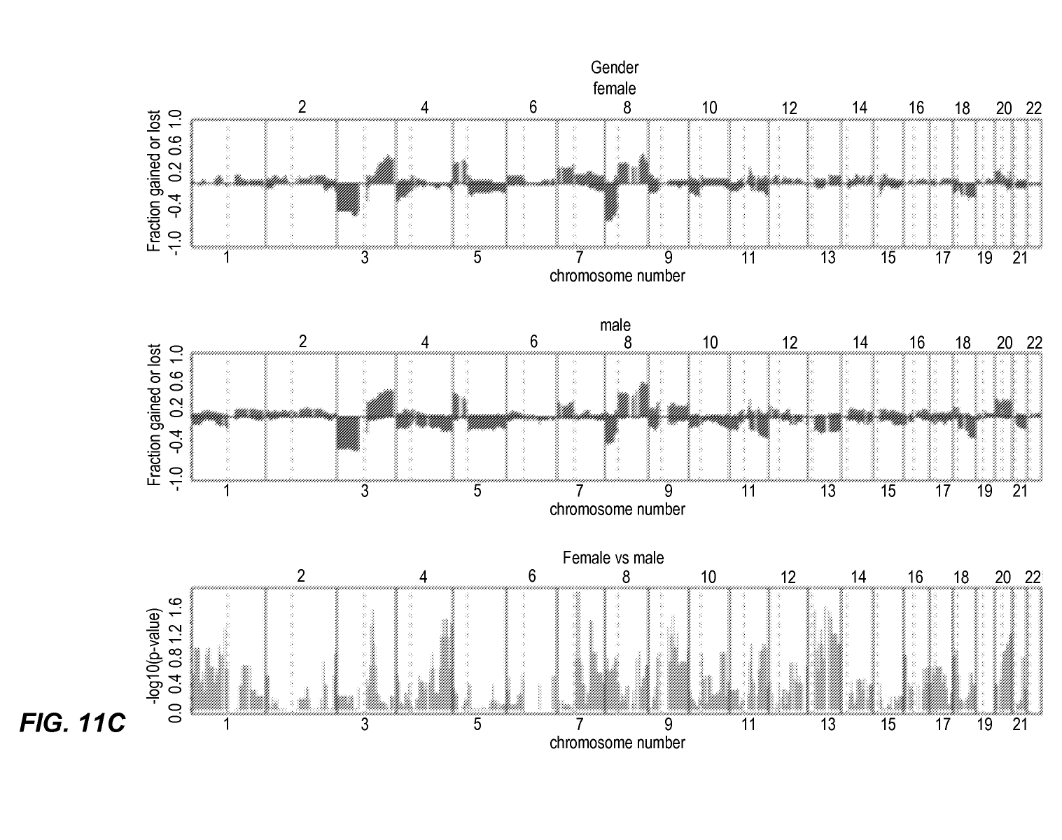

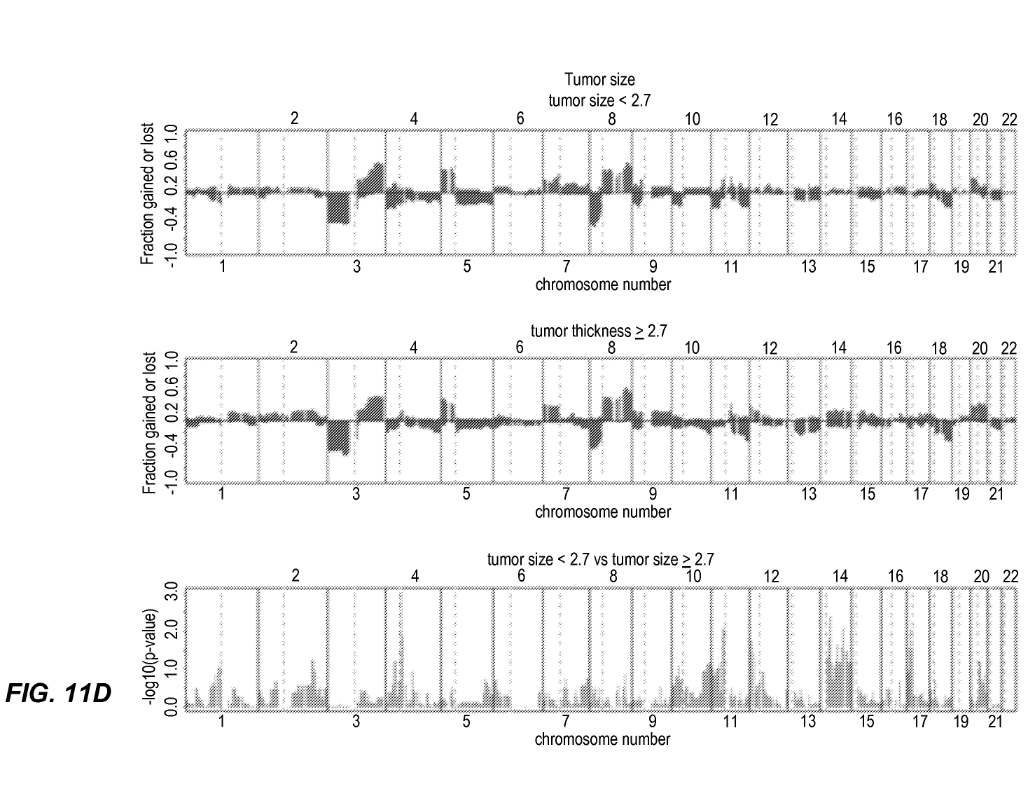

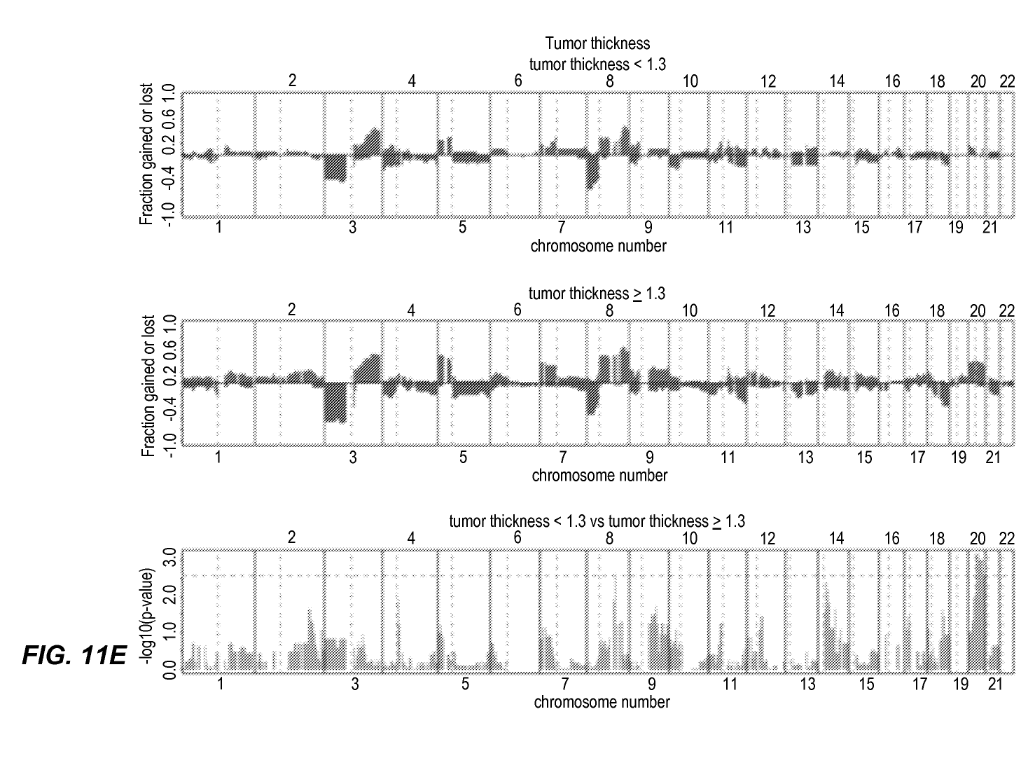

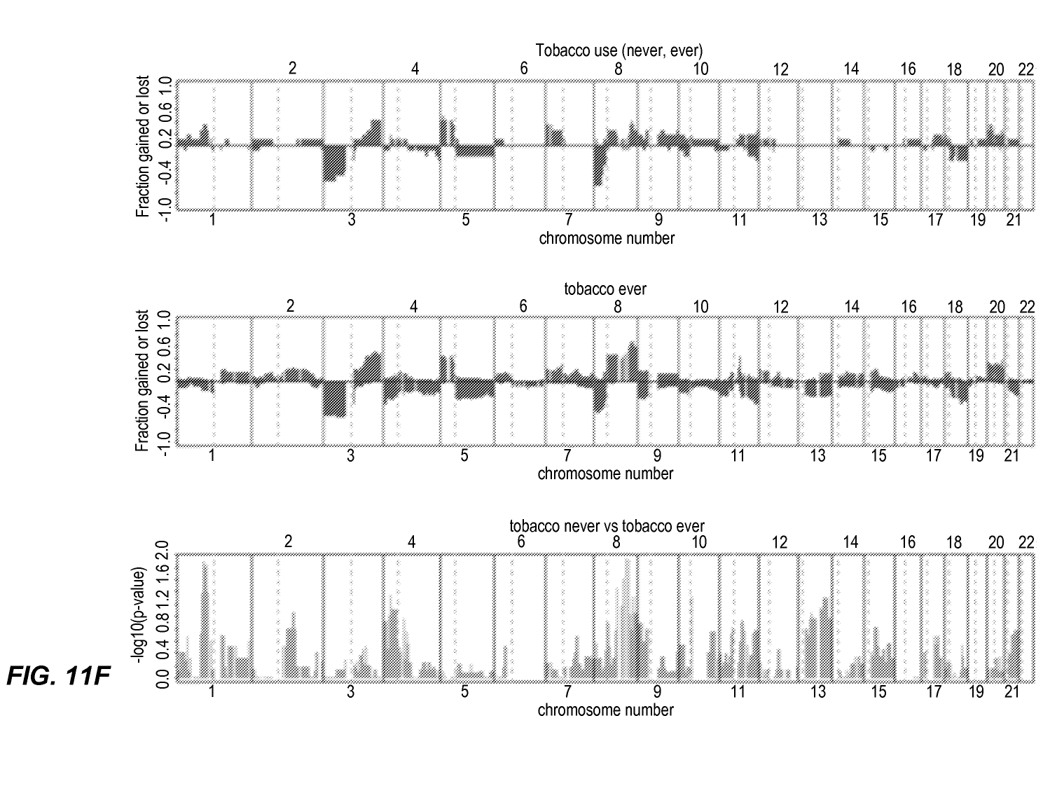

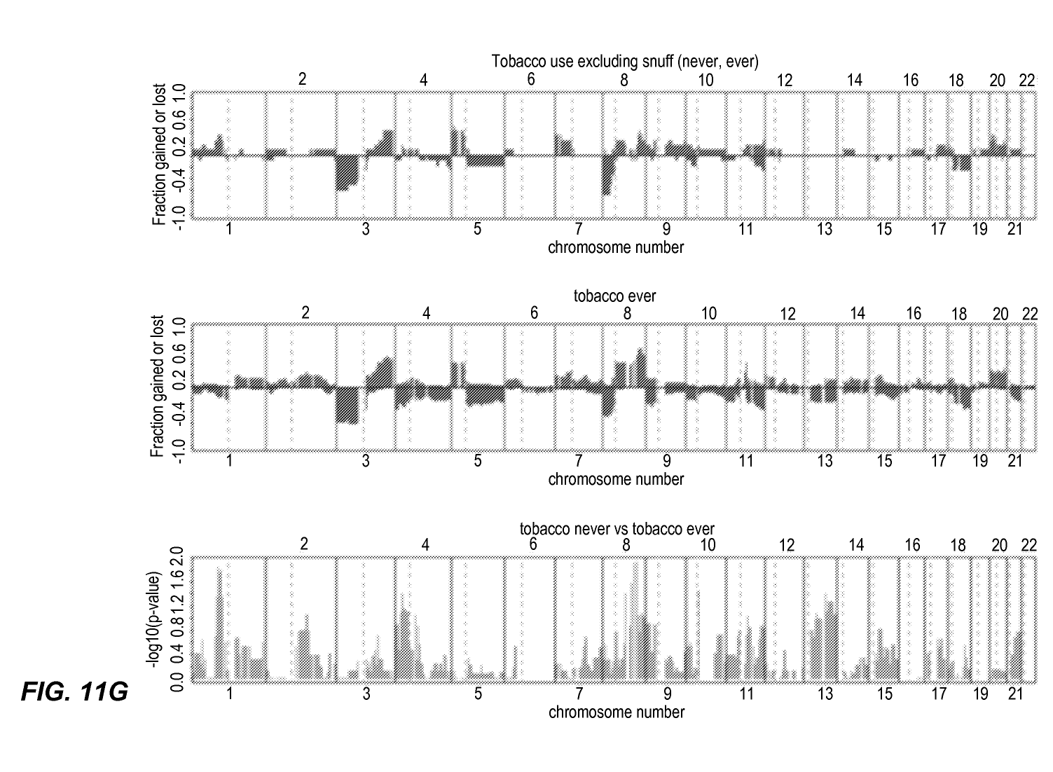

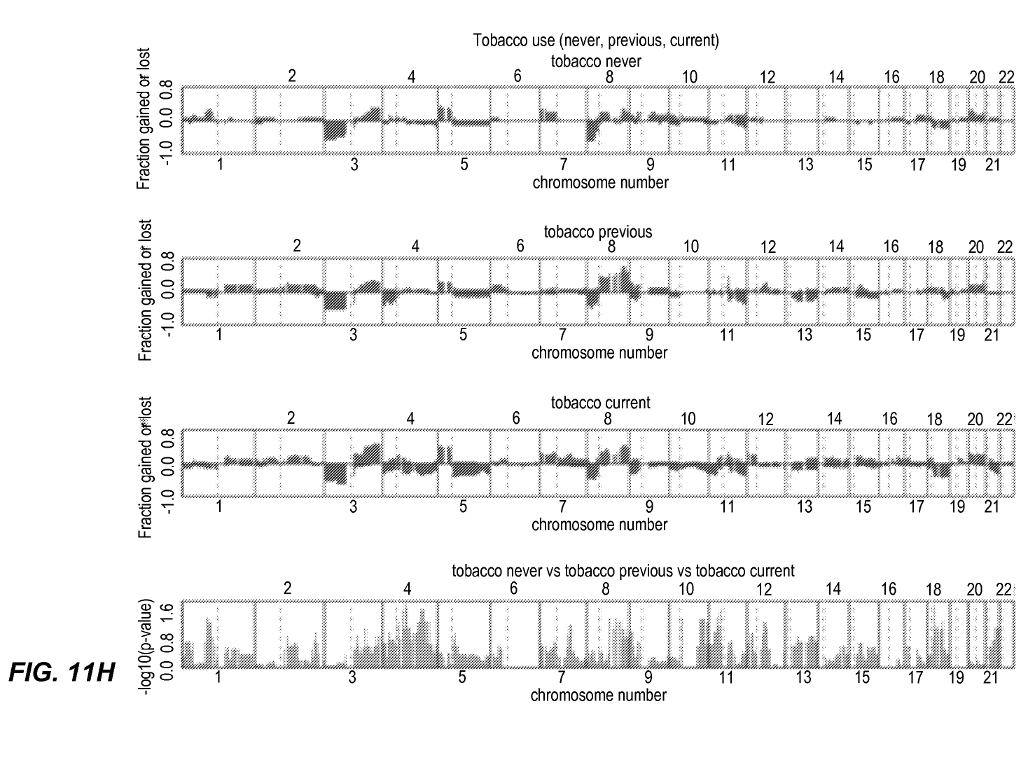

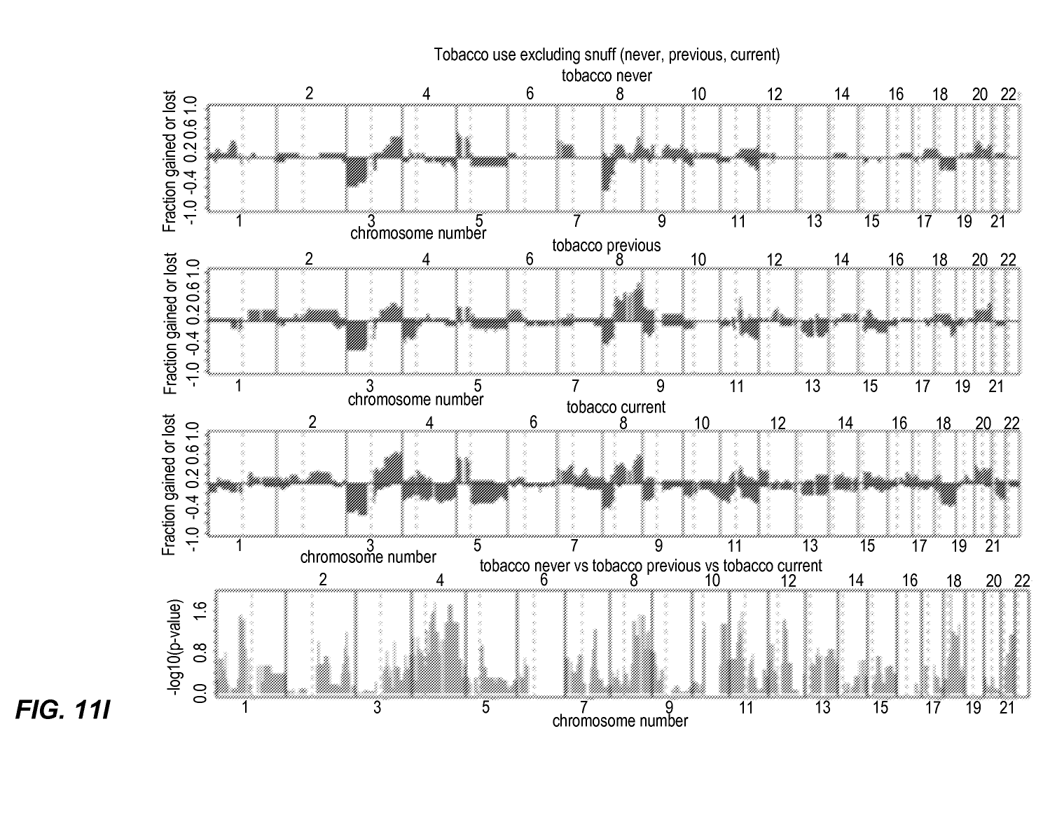

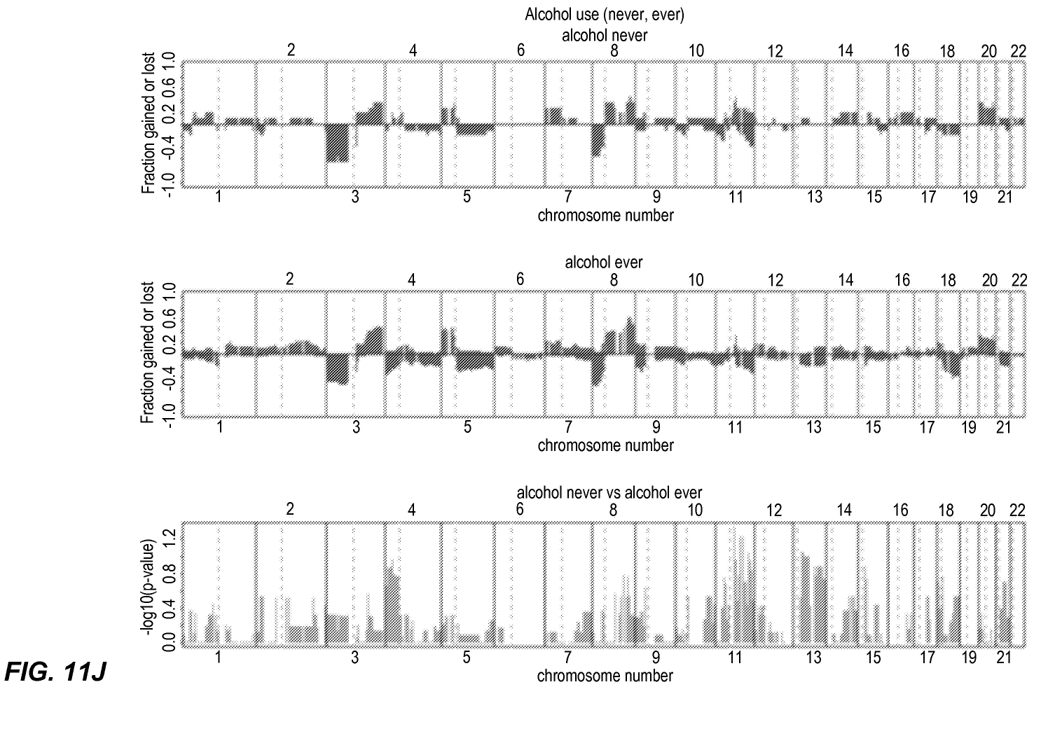

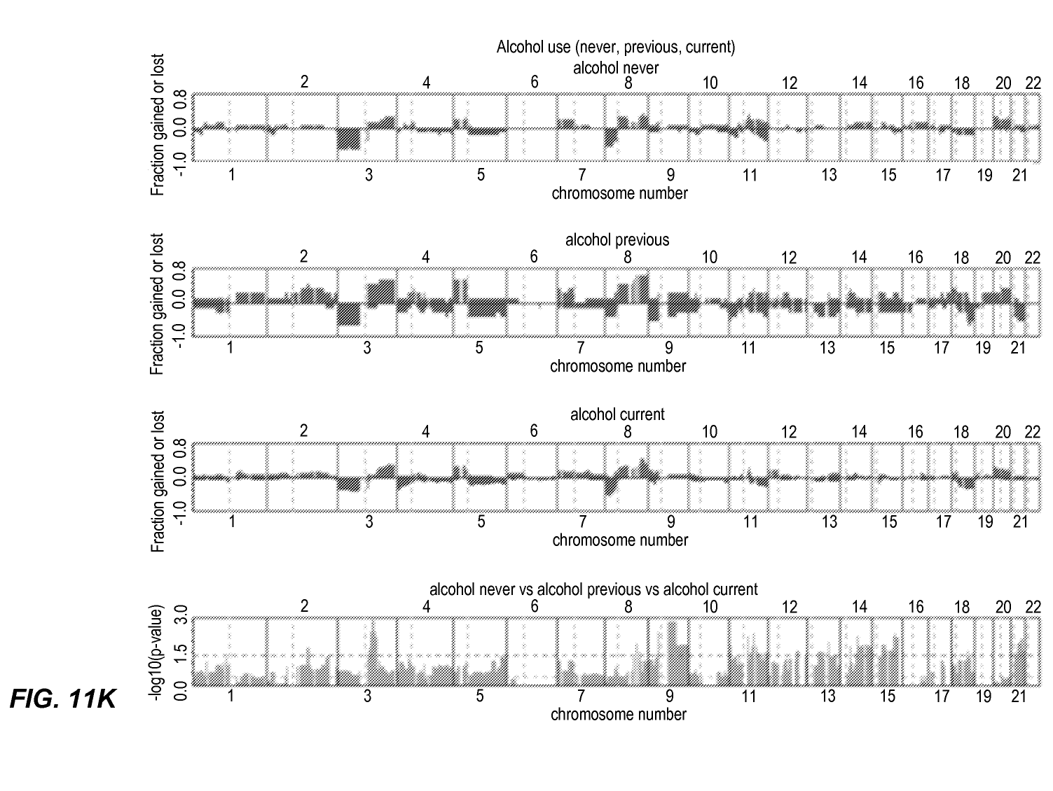

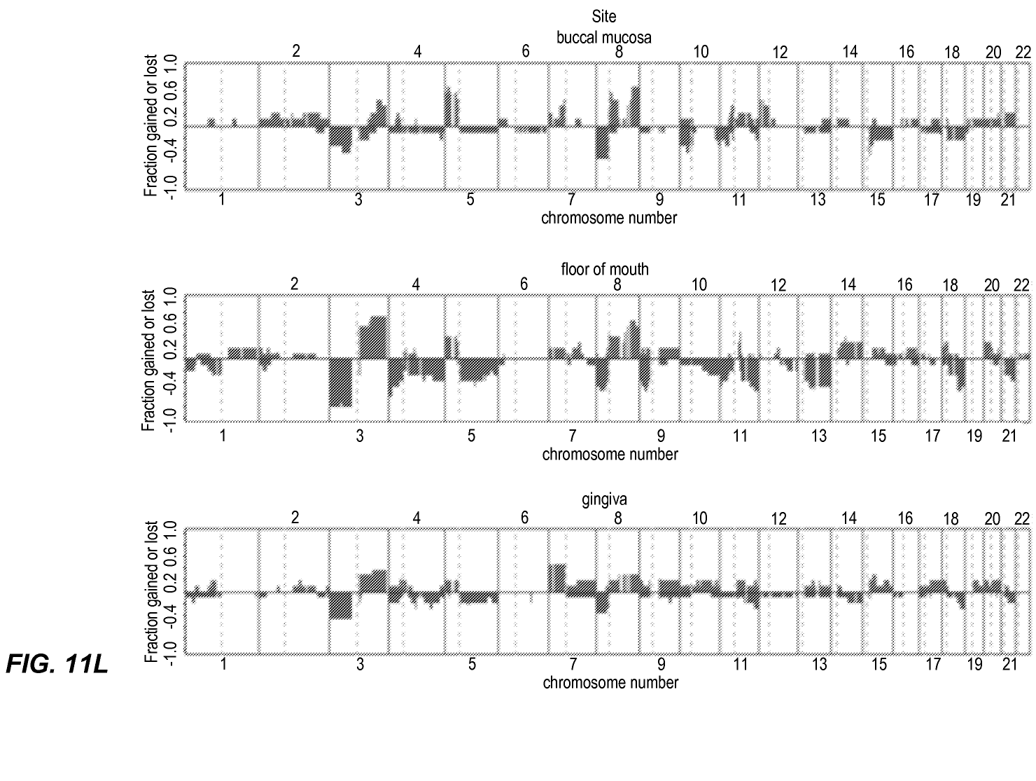

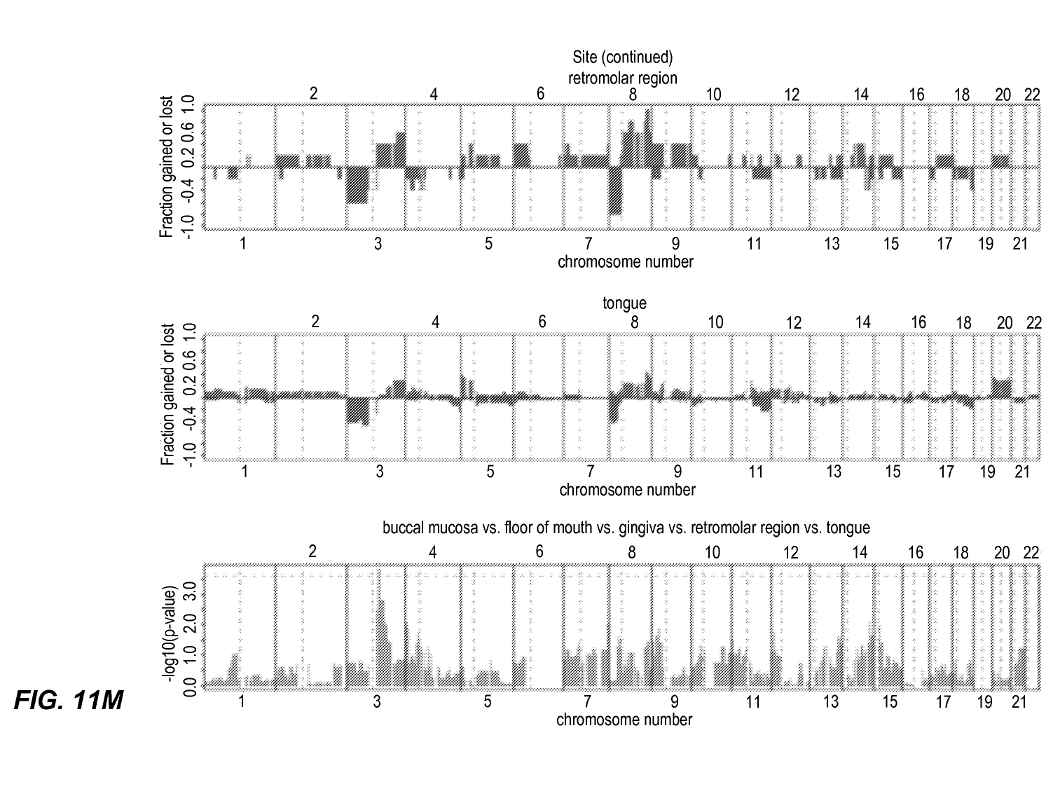

In addition, we observed previously described associations with positive nodal status (O'Brien et al. 1986; Whitehurst et al. 1977), including increased tumor size (p=0.018), tumor thickness (p=0.010) and reduced survival (Table 16 and FIG. 10), providing evidence that the clinical behavior of tumors in cohort#2 is similar to other oral SCC cohorts. We did not, however, identify individual copy number aberrations on a clone-wise basis that were significantly associated with clinical characteristics after correction for multiple testing (FIG. 11). In addition, we found only tumor size to be associated with nodal status amongst patients with 3q8pq20 tumors (Table 17). Assessment of other characteristics (e.g. gene expression signatures) will be required to determine if it is possible to further stratify 3q8pq20 patients for risk of metastasis.

TABLE 15
Association of clinical variables with genome instability characteristics

| | | | | | | No. | | No. | | | |
|---|---|---|---|---|---|---|---|---|---|---|---|
| | | | | No. Chrs. | | Chrs. | No. Whole | Whole | Fxn. | Fxn. | Fxn. |
| | | | Copy No. | with | No. of | with | Chr. | Arm | Genome | Genome | Genome |
| | | n | Transitions | Transitions | Amplifications | Amp. | Changes | Changes | Gained | Lost | Altered |
| Nodal status | | 63 | | | | | | | | | |
| N0 | | 40 | 26 | 11 | 0 | 0 | 7 | 9.5 | 0.036 | 0.050 | 0.103 |
| N+ | | 23 | 29 | 12 | 0 | 0 | 9 | 12 | 0.102 | 0.084 | 0.204 |
| | p-value | | 0.170 | 0.252 | 0.182 | 0.293 | 0.045 | 0.062 | 0.004 | 0.213 | 0.024 |
| Age | | 63 | | | | | | | | | |
| <65 | | 30 | 29 | 12 | 1.5 | 0.5 | 9.5 | 12.5 | 0.064 | 0.096 | 0.166 |
| ≧65 | | 33 | 26 | 11 | 0 | 0 | 7 | 9 | 0.064 | 0.046 | 0.112 |
| | p-value | | 0.429 | 0.263 | 0.170 | 0.135 | 0.241 | 0.191 | 0.727 | 0.081 | 0.319 |
| Gender | | 63 | | | | | | | | | |
| Female | | 26 | 29.5 | 11.5 | 0 | 0 | 8 | 9.5 | 0.036 | 0.050 | 0.109 |
| Male | | 37 | 26 | 12 | 0 | 0 | 8 | 11 | 0.065 | 0.083 | 0.161 |
| | p-value | | 0.800 | 0.854 | 0.591 | 0.451 | 0.710 | 0.576 | 0.466 | 0.174 | 0.309 |
| Tumor size | | 62 | | | | | | | | | |
| <2.7 cm | | 29 | 26 | 11 | 0 | 0 | 9 | 13 | 0.064 | 0.062 | 0.148 |
| ≧2.7 cm | | 33 | 29 | 12 | 0 | 0 | 8 | 11 | 0.066 | 0.064 | 0.161 |
| | p-value | | 0.713 | 0.243 | 0.991 | 0.914 | 0.886 | 0.952 | 0.695 | 0.811 | 0.930 |
| Tumor thickness | | 50 | | | | | | | | | |
| <1.3 cm | | 24 | 25.5 | 11 | 0 | 0 | 7 | 10 | 0.036 | 0.040 | 0.112 |
| ≧1.3 cm | | 26 | 29.5 | 12 | 0 | 0 | 8.5 | 12 | 0.102 | 0.084 | 0.207 |
| | p-value | | 0.308 | 0.258 | 0.568 | 0.427 | 0.110 | 0.088 | 0.019 | 0.105 | 0.051 |
| Tobacco use | | 46 | | | | | | | | | |
| never | | 12 | 28.5 | 11.5 | 0 | 0 | 6.5 | 8.5 | 0.048 | 0.054 | 0.104 |
| ever | | 34 | 29.5 | 12 | 1.5 | 0.5 | 8.5 | 12 | 0.064 | 0.090 | 0.166 |
| | p-value | | 0.670 | 0.474 | 0.354 | 0.407 | 0.146 | 0.172 | 0.228 | 0.107 | 0.083 |
| Tobacco use excluding | | 45 | | | | | | | | | |
| snuff | | | | | | | | | | | |
| never | | 12 | 28.5 | 11.5 | 0 | 0 | 6.5 | 8.5 | 0.048 | 0.054 | 0.104 |
| ever | | 33 | 29 | 12 | 3 | 1 | 9 | 12 | 0.064 | 0.096 | 0.171 |
| | p-value | | 0.708 | 0.479 | 0.316 | 0.330 | 0.132 | 0.165 | 0.246 | 0.088 | 0.084 |
| Tobacco use | | 46 | | | | | | | | | |
| never | | 12 | 28.5 | 11.5 | 0 | 0 | 6.5 | 8.5 | 0.048 | 0.054 | 0.104 |
| previous | | 14 | 27.5 | 11 | 4 | 1 | 8 | 10 | 0.036 | 0.067 | 0.129 |
| current | | 20 | 31 | 13 | 0 | 0 | 10 | 14 | 0.073 | 0.103 | 0.223 |
| | p-value | | 0.584 | 0.080 | 0.516 | 0.438 | 0.141 | 0.129 | 0.272 | 0.139 | 0.093 |
| Tobacco use excluding | | 45 | | | | | | | | | |
| snuff | | | | | | | | | | | |
| never | | 12 | 28.5 | 11.5 | 0 | 0 | 6.5 | 8.5 | 0.048 | 0.054 | 0.104 |
| previous | | 14 | 27.5 | 11 | 4 | 1 | 8 | 10 | 0.036 | 0.067 | 0.129 |
| current | | 19 | 31 | 13 | 0 | 0 | 10 | 15 | 0.067 | 0.110 | 0.242 |
| | p-value | | 0.595 | 0.052 | 0.552 | 0.474 | 0.088 | 0.119 | 0.278 | 0.095 | 0.084 |
| Alcohol use | | 43 | | | | | | | | | |
| never | | 11 | 26 | 11 | 3 | 1 | 6 | 9 | 0.064 | 0.057 | 0.112 |
| ever | | 32 | 29.5 | 12 | 0 | 0 | 8 | 11.5 | 0.065 | 0.080 | 0.166 |
| | p-value | | 0.195 | 0.133 | 0.768 | 0.801 | 0.172 | 0.243 | 0.421 | 0.263 | 0.200 |
| Alcohol use | | 43 | | | | | | | | | |
| never | | 11 | 26 | 11 | 3 | 1 | 6 | 9 | 0.064 | 0.057 | 0.112 |
| previous | | 7 | 29 | 11 | 0 | 0 | 10 | 13 | 0.233 | 0.180 | 0.335 |
| current | | 25 | 30 | 12 | 0 | 0 | 7 | 10 | 0.037 | 0.049 | 0.135 |
| | p-value | | 0.442 | 0.332 | 0.962 | 0.975 | 0.059 | 0.062 | 0.018 | 0.037 | 0.012 |
| Tumor site | | 57 | | | | | | | | | |
| Buccal mucosa | | 91 | 26 | 11 | 5 | 1 | 10 | 12 | 0.064 | 0.083 | 0.150 |
| Floor of mouth | | 11 | 26 | 11 | 0 | 0 | 9 | 13 | 0.109 | 0.130 | 0.259 |
| Gingiva | | 11 | 19 | 11 | 0 | 0 | 2 | 3 | 0.028 | 0.006 | 0.095 |
| Retromolar region | | 15 | 31 | 13 | 4 | 1 | 7 | 10 | 0.128 | 0.064 | 0.198 |
| Tongue | | 21 | 26 | 11 | 0 | 0 | 5 | 7 | 0.036 | 0.030 | 0.088 |
| | p-value | | 0.306 | 0.233 | 0.165 | 0.278 | 0.148 | 0.173 | 0.299 | 0.054 | 0.156 |

TABLE 16
Patient and tumor characteristics relative to cervical node status

| | | | | | | 95% | |
|---|---|---|---|---|---|---|---|
| | | | | | | confidence | |
| | | | | | Odds | interval | |
| | n | N0 | N+ | p-value | Ratio | lower | upper |
| 3q8pq20 status | 63 | | | 0.006 | 11.494 | 1.516 | 521.823 |
| 3q8pq20 | | 26 | 22 | | | | |
| non-3q8pq20 | | 1 | 14 | | | | |
| Age | 63 | | | 0.611 | 1.327 | 0.422 | 4.225 |
| <65 | | 18 | 12 | | | | |
| ≧65 | | 22 | 11 | | | | |
| Gender | 63 | | | 0.440 | 1.517 | 0.475 | 4.879 |
| Female | | 15 | 11 | | | | |
| Male | | 25 | 12 | | | | |
| Tumor size | 62 | | | 0.018 | 0.251 | 0.066 | 0.852 |
| <2.7 cm | | 23 | 6 | | | | |
| ≧2.7 cm | | 16 | 17 | | | | |
| Tumor thickness | 50 | | | 0.010 | 0.200 | 0.044 | 0.780 |
| <1.3 cm | | 19 | 5 | | | | |
| ≧1.3 cm | | 11 | 15 | | | | |
| Tobacco use | 46 | | | 1.000 | 0.918 | 0.167 | 4.365 |
| never | | 8 | 4 | | | | |
| ever | | 22 | 12 | | | | |
| Tobacco use excluding | 45 | | | 1.000 | 1.000 | 0.180 | 4.826 |
| snuff | | | | | | | |
| never | | 8 | 4 | | | | |
| ever | | 22 | 11 | | | | |
| Tobacco use | 46 | | | 0.923 | NA | NA | NA |
| never | | 8 | 4 | | | | |
| previous | | 10 | 4 | | | | |
| current | | 12 | 8 | | | | |
| Tobacco use excluding | 45 | | | 0.922 | NA | NA | NA |
| snuff | | | | | | | |
| never | | 8 | 4 | | | | |
| previous | | 10 | 4 | | | | |
| current | | 12 | 7 | | | | |
| Alcohol use | 43 | | | 0.494 | 0.556 | 0.080 | 2.910 |
| never | | 8 | 3 | | | | |
| ever | | 19 | 13 | | | | |
| Alcohol use | 43 | | | 0.751 | NA | NA | NA |
| never | | 8 | 3 | | | | |
| previous | | 4 | 3 | | | | |
| current | | 15 | 10 | | | | |
| Site | 57 | | | 0.496 | NA | NA | NA |
| buccal mucosa | | 6 | 3 | | | | |
| floor of mouth | | 6 | 5 | | | | |
| gingiva | | 8 | 3 | | | | |
| retromolar region | | 2 | 3 | | | | |
| tongue | | 16 | 5 | | | | |

TABLE 17
Patient and tumor characteristics of 3q8pq20 tumor subtype
relative to cervical node status

| | | | | | | 95% | |
|---|---|---|---|---|---|---|---|
| | | | | | | confidence | |
| | | 3q8pq20 | 3q8pq20 | p- | Odds | interval | |
| | n | N0 | N+ | value | Ratio | lower | upper |
| Age | 48 | | | 1.000 | 0.882 | 0.242 | 3.212 |
| <65 | | 15 | 12 | | | | |
| ≧65 | | 11 | 10 | | | | |
| Gender | 48 | | | 0.557 | 1.559 | 0.421 | 5.904 |
| Female | | 9 | 10 | | | | |
| Male | | 17 | 12 | | | | |
| Tumor size | 47 | | | 0.019 | 0.219 | 0.050 | 0.849 |
| <2.7 cm | | 16 | 6 | | | | |
| ≧2.7 cm | | 9 | 16 | | | | |
| Tumor | 39 | | | 0.054 | 0.248 | 0.048 | 1.101 |
| thickness | | | | | | | |
| <1.3 cm | | 12 | 5 | | | | |
| ≧1.3 cm | | 8 | 14 | | | | |
| Tobacco use | 39 | | | 1.000 | 1.194 | 0.195 | 6.892 |
| never | | 5 | 4 | | | | |
| ever | | 18 | 12 | | | | |
| Tobacco use | 38 | | | 1.000 | 1.300 | 0.210 | 7.596 |
| excluding | | | | | | | |
| snuff | | | | | | | |
| never | | 5 | 4 | | | | |
| ever | | 18 | 11 | | | | |
| Tobacco use | 39 | | | 1.000 | NA | NA | NA |
| never | | 5 | 4 | | | | |
| previous | | 7 | 4 | | | | |
| current | | 11 | 8 | | | | |
| Tobacco use | 38 | | | 1.000 | NA | NA | NA |
| excluding | | | | | | | |
| snuff | | | | | | | |
| never | | 5 | 4 | | | | |
| previous | | 7 | 4 | | | | |
| current | | 11 | 7 | | | | |
| Alcohol use | 36 | | | 0.709 | 0.699 | 0.091 | 4.441 |
| never | | 5 | 3 | | | | |
| ever | | 15 | 13 | | | | |
| Alcohol use | 36 | | | 0.901 | NA | NA | NA |
| never | | 5 | 3 | | | | |
| previous | | 4 | 3 | | | | |
| current | | 11 | 10 | | | | |
| Site | 37 | | | 0.863 | NA | NA | NA |
| buccal | | 4 | 3 | | | | |
| mucosa | | | | | | | |
| floor of | | 5 | 5 | | | | |
| mouth | | | | | | | |
| gingiva | | 3 | 2 | | | | |
| retromolar | | 2 | 3 | | | | |
| region | | | | | | | |
| tongue | | 10 | 5 | | | | |

### Discussion

By comparison of recurrent copy number alterations in oral pre-cancers and cancers, we have obtained evidence that there are at least two pathways of oral cancer development. One subtype acquires one or more of the aberrations +3q, −8p, +8q and/or +20 in dysplastic lesions, whereas recurrent copy number aberrations are absent from the other subtype. The 3q8pq20 subtype further subdivides according to levels of genome instability and alterations in methylation profiles. Notably, the two subtypes differ in clinical behavior, the non-3q8pq20 SCCs being associated with a very low risk for cervical node metastasis. Other lines of evidence supporting diverse routes to oral cancer (Hunter et al. 2005; Hunter et al. 2006; Jin et al. 2006; Noutomi et al. 2006) have highlighted differences in genome instability, gene expression profiles and possibly cell of origin as distinguishing features.

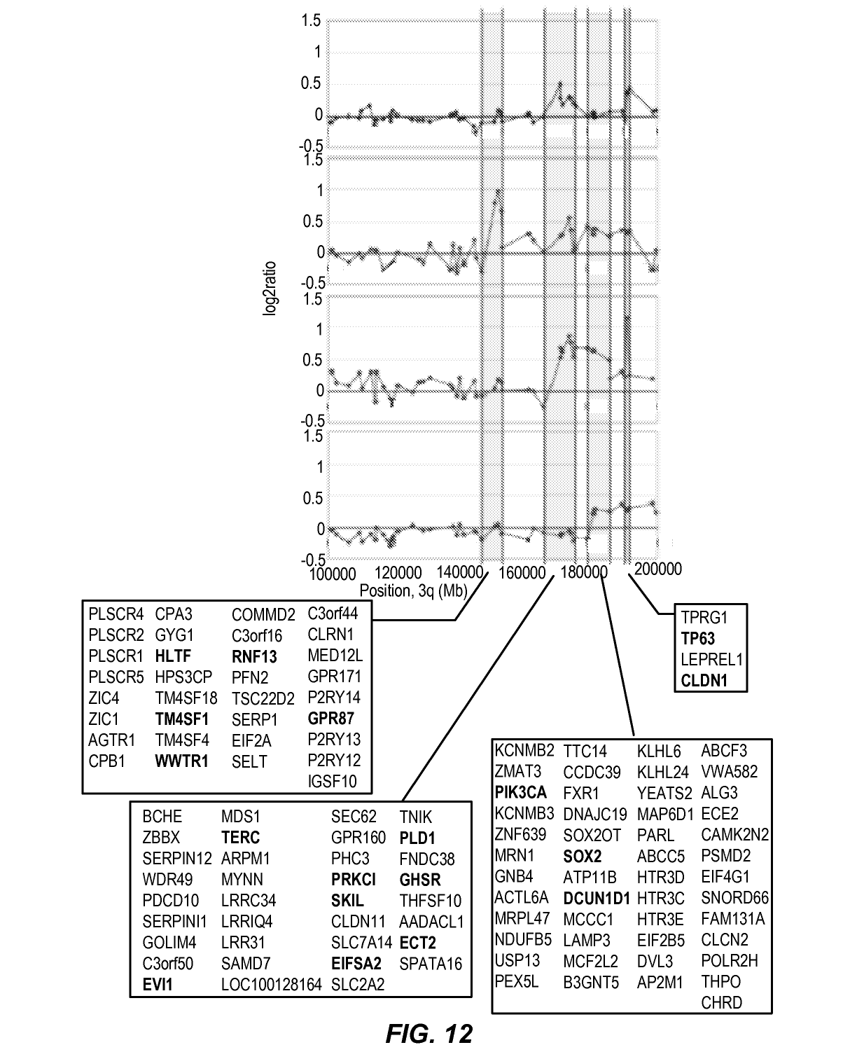

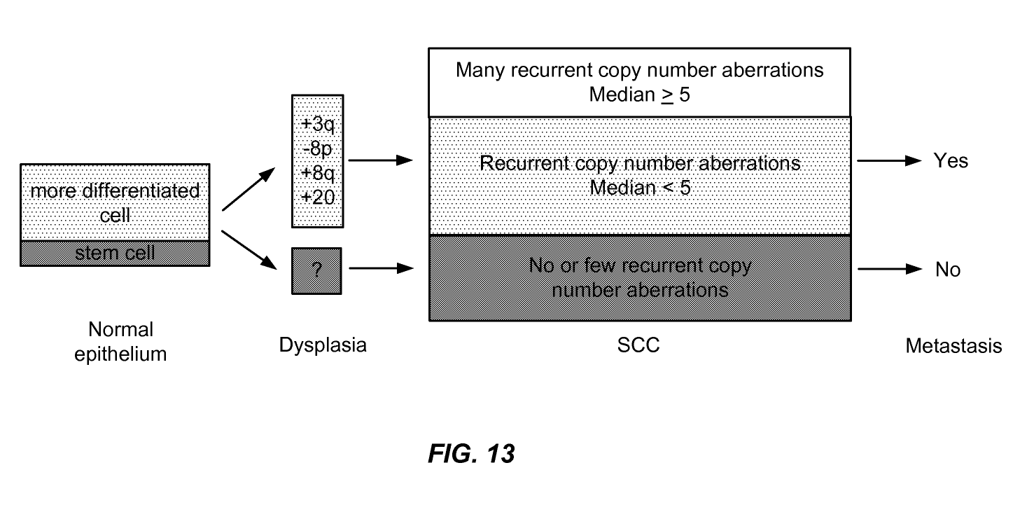

Our observations raise questions as to mechanism—the identity of the genes in these regions (3q, 8p, 8q and 20) and the functional consequences of their gain or loss that provide a growth advantage when at altered copy number early on in the pre-cancers (dysplasia). Identifying the genes from the copy number data alone is challenging, as the involved regions are large. Losses involving 8p and gains involving 3q, 8q and 20q occur frequently in cancers. Some insight into the genes that may be playing a role in de-regulating growth in pre-cancerous lesions may be obtained by considering candidate oncogenes and tumor suppressors that have been suggested for these regions based on finding that they are amplified or deleted in tumors. It is important to bear in mind, however, that candidate oncogenes mapping to regions of low level gains in pre-cancers may function differently than they do when at highly elevated copy number in tumors. Moreover, the ensemble of genes within these large regions (i.e. the balance of oncogenic and tumor suppressor functions) may together promote the pre-neoplastic changes. Nevertheless, taking this approach, JAG1 appears to be a likely candidate on chromosome 20p, as we found it to be amplified in dysplasia (Table 12) as well as cancer (Snijders et al. 2005). We also observed amplification at 20q11 in SCC cohort#1, suggesting BCL2L1, DNMT3B, E2F1, NCOA6, TGIF2 and ITCH as candidate oncogenes that could be contributing to the early de-regulation of growth. Similarly, candidate oncogenes on 8q identified in oral SCC include YWHAZ (Lin et al. 2009), MYC, PVT1 and associated miRNAs. Analysis of recurrent regions of amplification on 3q in our oral SCC cohorts found four regions, suggesting TM4SF1, WWTR1, RNF13, GPR87 (region 1), EV11, TERC, PRKCI, SKIL, EIF5A2, PLD1, GHSR, ECT2 (region 2), PIK3CA, SOX2, DCUN1D1 (region 3), TP63 and CLDN1 (region 4) as candidate oncogenes (FIG. 12).

Treatment for oral cancer is almost always surgical. Identification of patients with node-positive (N+) necks is the most important question to be accurately answered prior to surgical resection of the tumor, as well as for post-surgical treatment and follow-up (Cheng and Schmidt 2008). Typically, patients are assessed prior to surgery for lymph node metastases by palpation of the lymph nodes in the neck and by imaging (CT, MRI, PET scan). For patients with clinically node negative necks, treatment options include a “wait and see” approach or elective neck dissection (i.e. performing a neck dissection when there is no clinical or radiographic evidence of neck metastasis) if the chance of metastasis is >20% based on current risk assessment capability (Cheng et al. 2008). The 20% cutoff was established by mathematical modeling of the decisions and outcomes of management of the N0 neck to determine the threshold at which the benefits outweigh the costs of prophylactically treating the neck (Weiss et al. 1994). Currently, tumor thickness is considered the best predictor of metastasis. Since it is difficult to assess this parameter from the incisional biopsy prior to surgery (Cheng et al. 2008), the American Joint Commission on Cancer (AJCC) TNM staging protocol, which is based on surface diameter of the tumor (Byers et al. 1998) is often used to assess likelihood of metastasis. It is common in clinical practice to not recommend neck dissections if tumors are <2 cm in size (stage T1) and thickness <3 mm. Occult metastatic rates for oral SCC, however, are high and range from 20-45% for T1 tongue SCCs (Cheng et al. 2008). Thus, the failure to find evidence of metastasis on clinical exam provides little confidence that the patient does not require removal of the cervical lymph nodes. For this reason, in many medical centers, patients are routinely offered elective neck dissection (i.e. performing a neck dissection when there is no clinical or radiographic evidence of neck metastasis).

All patients in cohort#2 received neck dissections, as this treatment was a criterion for inclusion in the study. With the exception of three tumors, all were ≧3 mm in thickness. Tumor size of the 14 node negative non-3q8pq20 cases in this cohort ranged from 1.0-6.4 cm and thickness (recorded for seven cases) ranged from 0.2-1.3 cm (Table 3). None of the node negative non-3q8pq20 tumors would have met the criteria of stage T1 and thickness <3 mm for not recommending a neck dissection. In addition, two of the 14 node negative non-3q8pq20 cases were diagnosed as clinically node positive, but subsequently found to be node negative by pathology. Assessment of 3q8pq20 status prior to surgery would have added prognostic value and could have spared these 14 patients from unnecessary surgery. Moreover, our initial findings—non-3q8pq20 tumors have less than a 7% chance of metastasis—is well below the current 20% risk threshold, further supporting the potential utility of assessing 3q8pq20 status at the time of diagnostic biopsy to substantially improve clinical decisions regarding elective neck dissection.

We also find that FGG and FGA are correlated with risk for metastasis, although we did not find a clear cutpoint for either measure. Using cutpoints of 0.065 and 0.095 for FGG and FGA, respectively, we correctly identified more of the N0 cases than we did based on 3q8pq20 status; however more N+ cases are mistakenly called N0, which in the clinic may outweigh the benefits of detecting more N0 patients due to the extremely poor survival of patients who undergo surgical salvage for neck metastasis. Larger studies will be required to determine the utility of FGG, FGA and non-3q8pq20 subtype as biomarkers for cervical node status. For application in the clinic, however, it is likely that evaluation of 3q8pq20 (four loci) will have an advantage, since it would be more amenable to measurement using less complex biomarker assays (e.g. PCR) than would be assessment of genome-wide copy number alterations to determine FGG or FGA. Eliminating unnecessary neck dissections would reduce surgical risks, patient morbidity, lengthy surgeries (typically 10 hours) and hospitalization time.

There are a growing number of tumor types for which subtypes have been identified that lack copy number instability (Barretina et al. 2010; Fridlyand et al. 2006a; Smeets et al. 2009; Taylor et al. 2010). Better prognosis is often associated with these subtypes. In oral cancer, the non-3q8pq20 subtype is clearly a member of this group as there is low genomic instability and a low risk of metastasis. The driving force for these tumors remains obscure. The non-3q8pq20 oral tumors do not appear to have distinguishing methylation profiles or microsatellite instability, leaving open the possibility that there are underlying copy neutral chromosomal rearrangements or extensive mutations in oncogenes and tumor suppressors in this subtype. On the other hand, these tumors may be promoted by extrinsic factors that modify growth of epithelial cells, including inflammation and aberrant behavior of neighboring cells (Arwert et al. 2010). Infection with microorganisms is another candidate; bacteria have been reported in association with certain cancers (Fassi Fehri et al. 2011; Hooper et al. 2006), and also to modify growth signaling pathways in epithelial cells (Fassi Fehri et al. 2011; Hooper et al. 2009).

In summary, copy number analysis of oral cancers and pre-cancers has revealed two subtypes, 3q8pq20 and non-3q8pq20, distinguished by acquisition of specific copy number alterations in the early pre-cancerous lesions. The two subtypes are likely to develop by different pathways that result in tumors differing in their clinical behavior, namely risk for metastasis. In addition, we note that although much attention has focused on regions of genomic imbalance as biomarkers of progression because they are present at greater frequency in oral SCCs compared to pre-cancers (Bremmer et al. 2008), such markers, at best, can only report on the likelihood of progression of the 3q8pq20 subtype. They cannot provide information on progression of chromosomally stable non-3q8pq20 lesions.

### Example 2

### Assessment of DNA Copy Number by Array CGH from Brush Biopsies

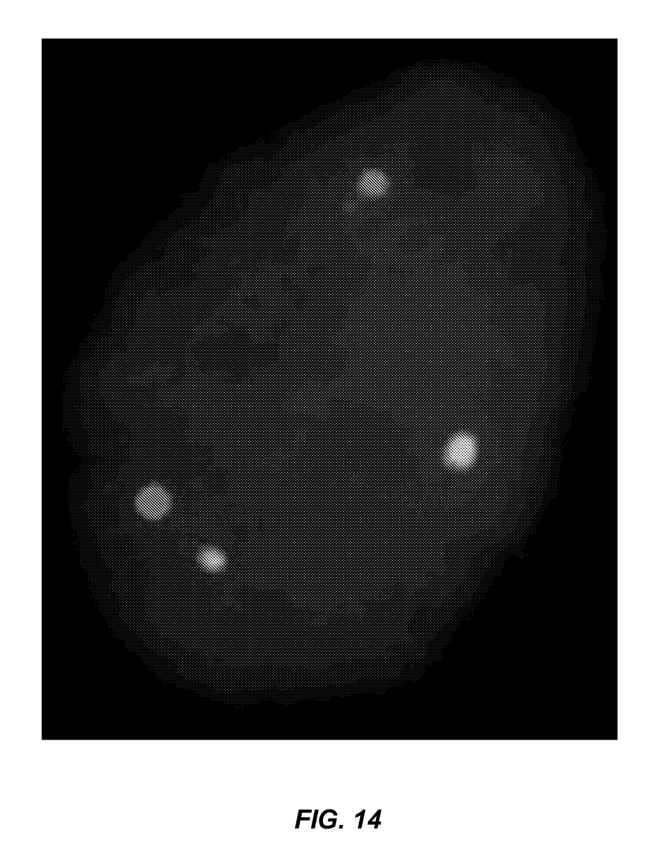

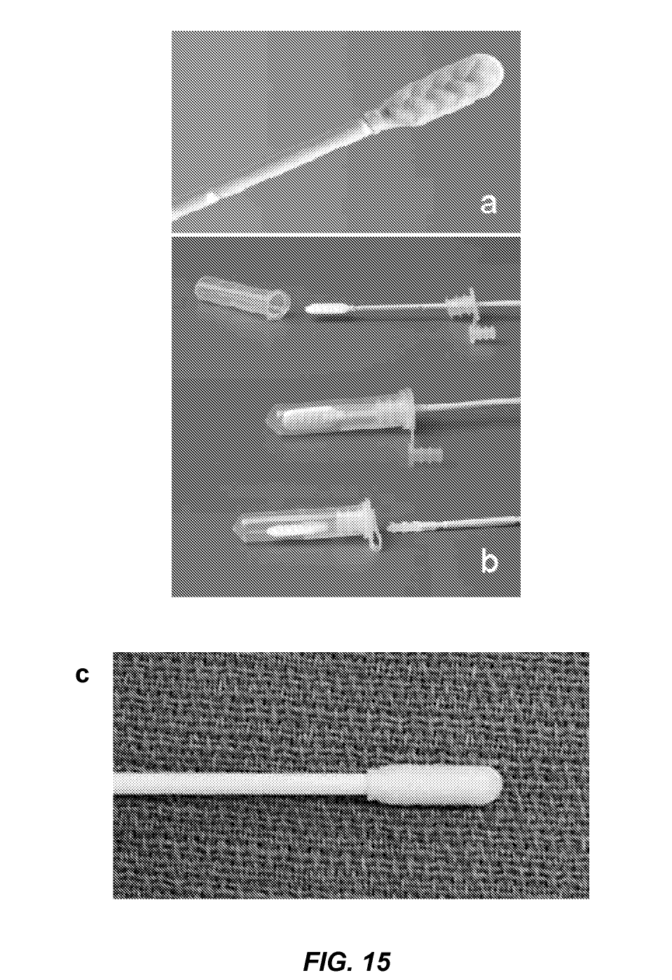

Brush biopsy sample analyses have employed DNA isolated from buccal swabs for PCR based assays (Garcia-Closas, et al., Cancer Epidemiol Biomarkers Prev, (2001) 10(6):687-96; and Mao, et al., Proc Natl Acad Sci USA, (1994) 91(21):9871-5) or cytological analyses using FISH on nuclei from cells smeared directly on glass slides and from fixed cell suspensions (FIG. 14). We have experience using the Oral CDx brush (Oral Scan Laboratories, Inc., Suffern, N.Y.), foam swabs (FIG. 15c) and the Isohelix swab (FIG. 15b). We prefer the Isohelix system, because the design of the swab will minimize bleeding (FIG. 15a), which could interfere with the measurement, and the tube and cap design (FIG. 15b) allow for easy release of the swab from the handle.

We have established that array CGH can be carried out with DNA isolated from oral brush biopsy samples. Our array CGH hybridizations typically use 0.5 μg of genomic DNA, although we have carried out this analysis with as little as 0.003 μg of DNA, and whole genome amplification methods currently allow analysis of only a few cells. Data in the literature indicate that 6 to 416 μg of DNA can be obtained by brush biopsy (London, et al, Cancer Epidemiol Biomarkers Prev (2001) 10:1227-30). Our experience using any of the brushes/swabs is consistent with this report. For example, two oral surgeons independently brush biopsied a 1×1 cm area of buccal mucosa with the foam brushes, yielding 1-1.3 μg of DNA following standard nucleic acid isolation procedures. Cytology of the brushing indicated that 100% of the harvested cells were epithelial.

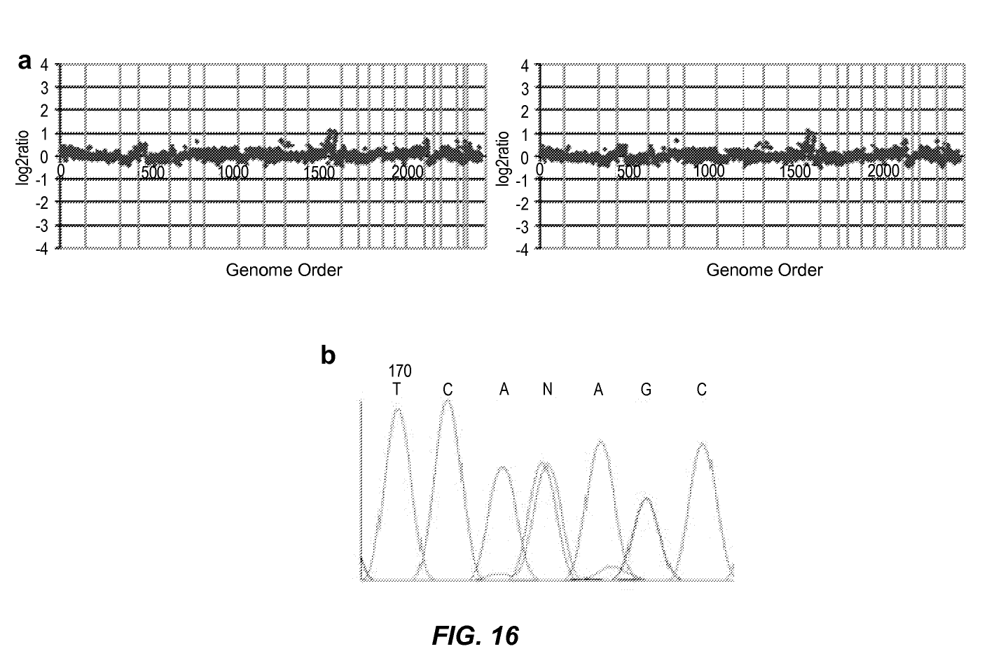

FIG. 16 shows that reproducible good quality array CGH data can be obtained from DNA isolated from independent brush biopsies of a lesion. In addition, we were able to determine that the tumor harbored a TP53 mutation (exon 5 codon 167 CAG to TAG, glutamine to stop) using Sanger sequencing. Both the array CGH and sequencing data indicate the brushings provide a sample with high tumor cell content.

Most recently, the lesions of two oral cancer patients, who were undergoing curative surgery for their cancers were swabbed using the Isohelix swab. The Isohelix DSK DNA isolation/stabilization buffer and proteinase K were added to the tube with the swab according to the manufacturer's instructions and shipped to UCSF. Using our standard laboratory protocol, we recovered 7.3 μg and 4.5 μg of DNA, respectively from the two samples that were suitable for array CGH.

### REFERENCES

Arwert, E. N., R. Lal, S. Quist, I. Rosewell, N. van Rooijen, and F. M. Watt. 2010. Tumor formation initiated by nondividing epidermal cells via an inflammatory infiltrate. Proc Natl Acad Sci USA 107: 19903-19908.
Barretina, J., B. S. Taylor, S. Banerji, A. H. Ramos, M. Lagos-Quintana, P. L. Decarolis, K. Shah, N. D. Socci, B. A. Weir, A. Ho et al. 2010. Subtype-specific genomic alterations define new targets for soft-tissue sarcoma therapy. Nat Genet 42: 715-721.
Benjamini, Y.a.H., Y. 1995. Controlling the false discovery rate: a practical and powerful approach to multiple testing. Journal of the Royal Statistical Society Series B 57: 289-300.
Bremmer, J. F., B. J. Braakhuis, A. Brink, M. A. Broeckaert, J. A. Belien, G. A. Meijer, D. J. Kuik, C. R. Leemans, E. Bloemena, I. van der Waal et al. 2008. Comparative evaluation of genetic assays to identify oral pre-cancerous fields. J Oral Pathol Med 37: 599-606.
Byers, R. M., A. K. El-Naggar, Y. Y. Lee, B. Rao, B. Formage, N. H. Terry, D. Sample, P. Hankins, T. L. Smith, and P. J. Wolf 1998. Can we detect or predict the presence of occult nodal metastases in patients with squamous carcinoma of the oral tongue? Head Neck 20: 138-144.
Califano, J., P. van der Riet, W. Westra, H. Nawroz, G. Clayman, S. Piantadosi, R. Corio, D. Lee, B. Greenberg, W. Koch et al. 1996. Genetic progression model for head and neck cancer: implications for field cancerization. Cancer Res 56: 2488-2492.
Chen, H. I., F. H. Hsu, Y. Jiang, M. H. Tsai, P. C. Yang, P. S. Meltzer, E. Y. Chuang, and Y. Chen. 2008. A probe-density-based analysis method for array CGH data: simulation, normalization and centralization. Bioinformatics 24: 1749-1756.
Cheng, A. and B. L. Schmidt. 2008. Management of the N0 neck in oral squamous cell carcinoma. Oral Maxillofac Surg Clin North Am 20: 477-497.
Couzin, J. and M. Schirber. 2006. Scientific misconduct. Fraud upends oral cancer field, casting doubt on prevention trial. Science 311: 448-449.
Fassi Fehri, L., T. N. Mak, B. Laube, V. Brinkmann, L. A. Ogilvie, H. Mollenkopf, M. Lein, T. Schmidt, T. F. Meyer, and H. Bruggemann. 2011. Prevalence of Propionibacterium acnes in diseased prostates and its inflammatory and transforming activity on prostate epithelial cells. Int J Med Microbiol 301: 69-78.
Fridlyand, J., A. M. Snijders, B. Ylstra, H. Li, A. Olshen, R. Segraves, S. Dairkee, T. Tokuyasu, B. M. Ljung, A. N. Jain et al. 2006a. Breast tumor copy number aberration phenotypes and genomic instability. BMC Cancer 6: 96.
Fridlyand, J., A. M. Snijders, B. Ylstra, H. Li, A. Olshen, R. Segraves, S. Dairkee, T. Tokuyasu, B. M. Ljung, A. N. Jain et al. 2006b. Breast tumor copy number aberration phenotypes and genomic instability. BMC Cancer 6: 96.
Garnis, C., R. Chari, T. P. Buys, L. Zhang, R. T. Ng, M. P. Rosin, and W. L. Lam. 2009. Genomic imbalances in precancerous tissues signal oral cancer risk. Mol Cancer 8: 50.
Gentleman, R. C., V. J. Carey, D. M. Bates, B. Bolstad, M. Dealing, S. Dudoit, B. Ellis, L. Gautier, Y. Ge, J. Gentry et al. 2004. Bioconductor: open software development for computational biology and bioinformatics. Genome Biol 5: R80.
Gillison, M. L. 2004. Human papillomavirus-associated head and neck cancer is a distinct epidemiologic, clinical, and molecular entity. Semin Oncol 31: 744-754.
Hermsen, M., A. Snijders, M. A. Guervos, S. Taenzer, U. Koerner, J. Baak, D. Pinkel, D. Albertson, P. van Diest, G. Meijer et al. 2005. Centromeric chromosomal translocations show tissue-specific differences between squamous cell carcinomas and adenocarcinomas. Oncogene 24: 1571-1579.
Herrero, R., X. Castellsague, M. Pawlita, J. Lissowska, F. Kee, P. Balaram, T. Rajkumar, H. Sridhar, B. Rose, J. Pintos et al. 2003. Human papillomavirus and oral cancer: the International Agency for Research on Cancer multicenter study. J Natl Cancer Inst 95: 1772-1783.
Hooper, S. J., S. J. Crean, M. A. Lewis, D. A. Spratt, W. G. Wade, and M. J. Wilson. 2006. Viable bacteria present within oral squamous cell carcinoma tissue. J Clin Microbiol 44: 1719-1725.
Hooper, S. J., M. J. Wilson, and S. J. Crean. 2009. Exploring the link between microorganisms and oral cancer: a systematic review of the literature. Head Neck 31: 1228-1239.
Hunter, K. D., E. K. Parkinson, and P. R. Harrison. 2005. Profiling early head and neck cancer. Nat Rev Cancer 5: 127-135.
Hunter, K. D., J. K. Thurlow, J. Fleming, P. J. Drake, J. K. Vass, G. Kalna, D. J. Higham, P. Herzyk, D. G. Macdonald, E. K. Parkinson et al. 2006. Divergent routes to oral cancer. Cancer Res 66: 7405-7413.
Ihaka, R. and R. Gentleman. 1996. R: a language for data analysis and graphics. Journal of Computational and Graphical Statistics 5: 299-314.
Jain, A. N., T. A. Tokuyasu, A. M. Snijders, R. Segraves, D. G. Albertson, and D. Pinkel. 2002. Fully automatic quantification of microarray image data. Genome Res 12: 325-332.
Jin, C., Y. Jin, J. Wennerberg, K. Annertz, J. Enoksson, and F. Mertens. 2006. Cytogenetic abnormalities in 106 oral squamous cell carcinomas. Cancer Genet Cytogenet 164: 44-53.
Lin, M., C. D. Morrison, S. Jones, N. Mohamed, J. Bacher, and C. Plass. 2009. Copy number gain and oncogenic activity of YWHAZ/14-3-3zeta in head and neck squamous cell carcinoma. Int J Cancer 125: 603-611.
MacDonald, D. G. and S. M. Saka. 1991. Structural indicators of the high risk lesion. Cambridge University Press, Cambridge.
Mehta, C.R.a.P., N. R. 1986. Algorithm 643. FEXACT: A Fortran subroutine for Fisher's exact test on unordered r*c contingency tables. ACM Transactions on Mathematical Software 12.
Miller, R. and D. Siegmund. 1982. Maximally Selected Chi Square Statistics. Biometrics 38: 1011-1016.
Noutomi, Y., A. Oga, K. Uchida, M. Okafuji, M. Ita, S. Kawauchi, T. Furuya, Y. Ueyama, and K. Sasaki. 2006. Comparative genomic hybridization reveals genetic progression of oral squamous cell carcinoma from dysplasia via two different tumourigenic pathways. J Pathol 210: 67-74.
O'Brien, C. J., J. W. Smith, S. J. Soong, M. M. Urist, and W. A. Maddox. 1986. Neck dissection with and without radiotherapy: prognostic factors, patterns of recurrence, and survival. Am J Surg 152: 456-463.
Olshen, A. B., E. S. Venkatraman, R. Lucito, and M. Wigler. 2004. Circular binary segmentation for the analysis of array-based DNA copy number data. Biostatistics 5: 557-572.
Paquette, J. and T. Tokuyasu. 2010. EGAN: exploratory gene association networks. Bioinformatics 26: 285-286.
Parkin, D. M., P. Pisani, and J. Ferlay. 1999. Global cancer statistics. CA Cancer J Clin 49: 33-64, 31.
Pitiyage, G., W. M. Tilakaratne, M. Tavassoli, and S. Warnakulasuriya. 2009. Molecular markers in oral epithelial dysplasia: review. J Oral Pathol Med 38: 737-752.
Poage, G. M., B. C. Christensen, E. A. Houseman, M. D. McClean, J. K. Wiencke, M. R. Posner, J. R. Clark, H. H. Nelson, C. J. Marsit, and K. T. Kelsey. 2010. Genetic and epigenetic somatic alterations in head and neck squamous cell carcinomas are globally coordinated but not locally targeted. PLoS One 5: e9651.
Schmidt, B. L., E. J. Dierks, L. Homer, and B. Potter. 2004. Tobacco smoking history and presentation of oral squamous cell carcinoma. J Oral Maxillofac Surg 62: 1055-1058.
Shaw, R. J., G. L. Hall, D. Lowe, T. Liloglou, J. K. Field, P. Sloan, and J. M. Risk. 2008. The role of pyrosequencing in head and neck cancer epigenetics: correlation of quantitative methylation data with gene expression. Arch Otolaryngol Head Neck Surg 134: 251-256.
Shiboski, C. H., B. L. Schmidt, and R. C. Jordan. 2005. Tongue and tonsil carcinoma: increasing trends in the U.S. population ages 20-44 years. Cancer 103: 1843-1849.
Silverman, S. J. 1998. Epidemiology. D.C. Decker Inc.
Smeets, S. J., R. H. Brakenhoff, B. Ylstra, W. N. van Wieringen, M. A. van de Wiel, C. R. Leemans, and B. J. Braakhuis. 2009. Genetic classification of oral and oropharyngeal carcinomas identifies subgroups with a different prognosis. Cell Oncol 31: 291-300.
Snijders, A. M., J. Fridlyand, D. A. Mans, R. Segraves, A. N. Jain, D. Pinkel, and D. G. Albertson. 2003. Shaping of tumor and drug-resistant genomes by instability and selection. Oncogene 22: 4370-4379.
Snijders, A. M., N. Nowak, R. Segraves, S. Blackwood, N. Brown, J. Conroy, G. Hamilton, A. K. Hindle, B. Huey, K. Kimura et al. 2001. Assembly of microarrays for genome-wide measurement of DNA copy number. Nat Genet 29: 263-264.
Snijders, A. M., B. L. Schmidt, J. Fridlyand, N. Dekker, D. Pinkel, R. C. Jordan, and D. G. Albertson. 2005. Rare amplicons implicate frequent deregulation of cell fate specification pathways in oral squamous cell carcinoma. Oncogene 24: 4232-4242.
Taylor, B. S., N. Schultz, H. Hieronymus, A. Gopalan, Y. Xiao, B. S. Carver, V. K. Arora, P. Kaushik, E. Cerami, B. Reva et al. 2010. Integrative Genomic Profiling of Human Prostate Cancer. Cancer Cell 18: 11-22.
Weiss, M. H., L. B. Harrison, and R. S. Isaacs. 1994. Use of decision analysis in planning a management strategy for the stage N0 neck. Arch Otolaryngol Head Neck Surg 120: 699-702.
Whitehurst, J. O. and C. A. Droulias. 1977. Surgical treatment of squamous cell carcinoma of the oral tongue: factors influencing survival. Arch Otolaryngol 103: 212-215.

### Sequences

Positions of STS markers are determined using both full sequences and primer information. Full sequences are aligned using blat, while is PCR (Jim Kent) and ePCR are used to find locations using primer information. Both sets of placements are combined to give final positions. In nearly all cases, full sequence and primer-based locations are in agreement, but in cases of disagreement, full sequence positions are used. Sequence and primer information for the markers were obtained from the primary sites for each of the maps and from UniSTS.

AV42101E7 (sts for RP11-72E23)
Alignment of dbSTS_15597 and chr3:145069908-145270175
(SEQ ID NO: 1)
AGGTCCTCAT AGTGGAGACG tgctgataat aaattcactc ccagaaaaaa 145169993
agtccccatc ctgattattt ccctaattag cactggaagg tcaaattaag 145170043
ggaaaaaatg tatacacaca cacacacaca cacacacaca cacacacaca 145170093
catcctacca aatcatacct ttaactaGGA GTTTACCTCC TAGGCATgcc 145170143
UniSTS
Forward primer:
(SEQ ID NO: 2)
AGGTCCTCATAGTGGAGACG
Reverse primer:
(SEQ ID NO: 3)
ATGCCTAGGAGGTAAACTCC
PCR product size: 177-201 (bp), Homo sapiens
GenBank Accession: Z23589
SHGC-1948 (D8S529, RH526) sts for RP11-184M21
Alignment of dbSTS_55247 and chr8:133999084-134199475
(SEQ ID NO: 4)
aataacctaa aatcctaaat gtaattagca tgctggcatt gaaaacaatc 134099208
ttgtaaataa ataagtaatg atacagaatg atatcgacaa tggattgtta 134099258
GGTGAAAAAG ATAGGCTCAA aaacaatatg ctgtatagat ttcctgaata 134099308
tatgtttaca cacacacaca cacacacaca cacacacaca cacacacaca 134099358
cacacacacg gaagagacat attggaGGCT AATAGCATTT AACAGTGGtt 134099408
ttctttgatt gggaggatta tgtgtaattt taattttctt tgtttcgttc 134099458
acttgttttg tctatttggg gactgctatt tttgctttaa aaattgca
UniSTS
Forward primer:
(SEQ ID NO: 5)
GGTGAAAAAGATAGGCTCAA
Reverse primer:
(SEQ ID NO: 6)
CCACTGTTAAATGCTATTAGCC
PCR product size: 142 (bp), Homo sapiens
GenBank Accession: Z23840
SHGC-1962 (AFM304ze9) sts for RP11-252K12
Alignment of dbSTS_26467 and chr8:10781516-10981913
(SEQ ID NO: 7)
gttctgtcat agctccattt cactaataag gagacagatg tggaggttgg 10881874
ggagttggtc ccaggtcacc caactgggga gggcagaggt tggggaggga 10881824
CAGGAGTCAA TAACCCAaag tcatgaaatg agaaaggaag taaacacttg 10881774
gatggagaat cacacacaca cacacacaca cacacacaca cacacacaca 10881724
cacacacacc tcctaacagg tatgttgtct gcaacaaggc aaaaataatt 10881674
cattaatatc tcatttaaac ttgagggcga gggaattcct gaaccacctc 10881624
tctggagcaa ataatggaaa ttggaaattg attgtcattt acctttgagg 10881574
aaGACTTCGG GATGTGCCAt gtctttggta tagggctgcg tggtgttgtg 10881524
acgcatgtga agaaatacat ccaaggacct tcctaagctc atctgcagcc 10881474
acaattcccc caccctatt
UniSTS
Forward primer:
(SEQ ID NO: 8)
CCCAAAGTCATGAAATGAGA
Reverse primer:
(SEQ ID NO: 9)
ACAACATACCTGTTAGGAGGTG
PCR product size: 103 (bp), Homo sapiens
GenBank Accession: Z24258

### H. Sapiens (D8S550) DNA Segment Containing (CA) Repeat; Clone AFM304Ze9; Single Read

### GenBank: Z24258.1

LOCUS Z24258 386 bp DNA linear PRI 28-Nov.-1994
DEFINITION H. sapiens (D8S550) DNA segment containing (CA) repeat; clone AFM304ze9; single read.
ACCESSION Z24258
VERSION Z24258.1 GI:394458
KEYWORDS CA repeat; dinucleotide repeat; GT repeat; microsatellite DNA; microsatellite marker; repeat polymorphism.
SOURCE Homo sapiens (human)
   ORGANISM Homo sapiens
      Eukaryota; Metazoa; Chordata; Craniata; Vertebrata; Euteleostomi;
      Mammalia; Eutheria; Euarchontoglires; Primates; Haplorrhini; Catarrhini; Homimidae; Homo.
REFERENCE 1
   AUTHORS Gyapay, G., Morissette, J., Vignal, A., Dib, C., Fizames, C., Millasseau, P., Marc, S., Bernardi, G., Lathrop, M. and Weissenbach, J.
   TITLE The 1993-94 Genethon human genetic linkage map
   JOURNAL Nat. Genet. 7 (2 SPEC NO), 246-339 (1994)
      PUBMED 7545953
REFERENCE 2 (bases 1 to 386)
   AUTHORS Weissenbach, J.
   TITLE Direct Submission
   JOURNAL Submitted (12-JUL-1993) Genethon, B. P. 60, 91002 Evry Cedex France.
      E-mail: Jean.Weissenbach@genethon.fr
COMMENT cloning vector is M13 mp 18ASBB;
   full automatic.
FEATURES Location/Qualifiers
   source 1.386
      /organism=“Homo sapiens”
      /mol_type=“genomic DNA”
      /db_xref=“taxon:9606”
      /chromosome=“8”
      /cell_line=“CEPH 134702”
      /clone_lib=“genomic DNA”

```
ORIGIN
(SEQ ID NO: 10)
    1 agctccattt cactaataag gagacagatg tggaggttgg ggagttggtc ccaggtcacc
  61 caactgggga gggcagaggt tggggaggga caggagtcaa taacccaaag tcatgaaatg
121 agaaaggaag taaacacttg gntggagant cacacacaca cacacacaca cacacacaca
181 cacacacctc ctaacaggta tgttgtctgc aacaaggcaa aaataattca ttaatatctc
241 atttaaactt gagggcgagg gaattcctga accacctctc tggagcaaat aatggaaatt
301 ggaaattgat tgtcatttac ctttgaggaa gacttcggga tgtgccatgt ctttggtata
361 gggctgcgtg gtgttgtgac gcatgt
SHGC-32354 (sts for RP11-258B14, 8g12)
Alignment of dbSTS_21453 and chr8:61001531-61201900
(SEQ ID NO: 11)
atttctatga cttagatatt ctgcatcaca aaatccctcc aaactgggac 61101500
tatgtttttg aagtcattca ttttacaatt ataacaacaa taacaataat 61101550
ATTTATTGTT TGCTTTGTGC CAggtactct actgctttac ataaattatc 61101600
tcattctgtc acatctaacg gcaactaagt atacgcttac atctgctagt 61101650
GGCACCTAAA ATAAGGATAT TGTTGgtcat ctttaaagaa atgtcttaac 61101700
ataccaaagt agtggaatca atagaataaa atatttaagt cttacaaagc 61101750
gtacgacact aaagtaatat aggat
Forward primer:
(SEQ ID NO: 12)
ATTTATTGTTTGCTTTGTGCCA
Reverse primer:
(SEQ ID NO: 13)
CAACAATATCCTTATTTTAGGTGCC
PCR product size: 125 (bp), Homo sapiens
GenBank Accession: G29372 Z39364
```

Positions of Flanking BACs that have been Sequenced

This is the only location found for RP11-72E23 (sts AFM210VE7)

### Complete Sequence

### GenBank: AC016967.24

Chromosome: chr3

### Start: 145059218

### End: 145206687

### Length: 147470

### Strand: +

### Score: 1000

### Band: 3q24

### This is the Only Location Found for RP11-252K12 (sts SHGC-1962)

BAC end sequence
End-Sequence Information

| GenBank Accession | Seqlen (bp) | Repeat | Hit | End |
|---|---|---|---|---|
| AZ517461 | 556 | No | Yes | SP6 |
| AQ491832 | 553 | No | Yes | T7 |

BAC end sequences are placed on the assembled sequence using Jim Kent's blat program
Chromosome: chr8

### Start: 10855865

### End: 11035922

### Length: 180058

### Strand: −

### Score: 1000

### Band: 8p23.1

### RP11-258B14 (sts SHGC-32354) BAC not Sequenced

### This is the Only Location Found for RP11-184M21 (sts SHGC-1948)

### Working Draft Sequence (6 Unordered Pieces)

### GenBank: AC090798.2

Chromosome: chr8

### Start: 134006900

### End: 134150078

### Length: 143179

### Strand: −

### Score: 1000

### Band: 8q24.22

It is understood that the examples and embodiments described herein are for illustrative purposes only and that various modifications or changes in light thereof will be suggested to persons skilled in the art and are to be included within the spirit and purview of this application and scope of the appended claims. All sequence references, publications, patents, and patent applications cited herein are hereby incorporated by reference in their entirety for all purposes.

1. A method of determining the presence of oral squamous cell carcinoma that is unlikely to metastasize in an oral sample from a subject, the method comprising determining relative copy numbers in sample DNA for the following chromosomal regions: 3q, 8p, 8q, and 20, wherein no gain of chromosomal regions 3q, 8q, and 20, and no loss of chromosomal region 8p is indicative of oral squamous cell carcinoma that is unlikely to metastasize.

2. The method of claim 1, wherein the method comprises determining relative copy numbers in sample DNA for the following chromosomal regions: 3q24-qter, 8pter-p23.1, 8q12-q24.2, and 20pter-qter, wherein no gain of chromosomal regions 3q24-qter, 8q12-q24.2, and 20pter-qter and no loss of chromosomal region 8pter-p23.1 is indicative of oral squamous cell carcinoma that is unlikely to metastasize.

3. A method of determining the presence of oral squamous cell carcinoma having a substantial likelihood of metastasis in a biological sample from a subject, the method comprising determining relative copy numbers in sample DNA for the following chromosomal regions: 3q, 8p, 8q, and 20, wherein a gain of one or more of chromosomal regions 3q, 8q, and 20, and/or a loss of chromosomal region 8p is indicative of oral squamous cell carcinoma having a substantial likelihood of metastasis.

4. The method of claim 3, wherein the method comprises determining relative copy numbers in sample DNA for the following chromosomal regions: 3q24-qter, 8pter-p23.1, 8q12-q24.2, and 20pter-qter, wherein a gain of one or more of chromosomal regions 3q24-qter, 8q12-q24.2, and 20pter-qter and/or a loss of chromosomal region 8pter-p23.1 is indicative of oral squamous cell carcinoma having a substantial likelihood of metastasis.

5. The method of claim 3, further comprising determining the presence of one or more genetic alterations selected from the group consisting of: fraction of genome gained (FGG), fraction of genome altered (FGA), altered methylation status, TP53 mutation(s), and the presence of relative copy number alterations at one or more loci other than 3q24-qter, 8pter-p23.1, 8q12-q24.2, and 20pter-qter, wherein the presence of one or more of said genetic alterations indicates an increased likelihood that metastasis will occur or has occurred.

6. The method of claim 3, further comprising determining one or more clinical parameters selected from the group consisting of tumor size, tumor thickness, tumor stage, the presence of metastasis by radiographic imaging, and lymph node status.

7. The method of claim 1, wherein chromosomal region:
3q24-qter extends from SEQ ID NO:1 to the q terminus of chromosome 3;
8pter-p23.1 extends from the p terminus of chromosome 8 to SEQ ID NO:7; and
8q12-q24.2 extends from SEQ ID NO:11 to SEQ ID NO:4.

8. The method of claim 1, wherein relative copy numbers are determined by analyzing genomic DNA.

9. The method of claim 1, wherein relative copy numbers are determined by analyzing RNA, cDNA, or DNA amplified from RNA.

10. The method of claim 1, wherein the method additionally comprises querying the copy number(s) of one or more control chromosomal regions.

11. The method of claim 1, wherein the method comprises:
contacting sample DNA with a combination of probes for chromosomal regions 3q, 8p, 8q, and 20;
incubating the probes with the sample under conditions in which each probe binds selectively with a nucleic acid sequence in its target chromosomal region to form a stable hybridization complex; and
detecting hybridization of the probes to determine copy number for each chromosomal region.

12. The method of claim 1, wherein the method is carried out by hybridization of sample nucleic acids to said combination of probes, which are immobilized on a substrate.

13. The method of claim 12, wherein the method is carried out by array comparative genomic hybridization (aCGH).

14. The method of claim 12, wherein the combination of probes comprises a plurality of probes for each chromosomal region.

15. The method of claim 14, wherein the combination of probes comprises a plurality of probes for each of one or more control chromosomal regions.

16. The method of claim 1, wherein the method is carried out by in situ hybridization, and each probe in the probe combination is labeled with a different label.

17. The method of claim 1, wherein the probe combination comprises at least 4, but not more than 100 probes.

18. The method of claim 17, wherein the probe combination comprises at least 4, but not more than 10 probes.

19. The method of claim 1, wherein the method comprises amplification of target nucleic acids in chromosomal regions 3q, 8p, 8q, and 20.

20. The method of claim 19, wherein the method comprises polymerase chain reaction (PCR) or multiplex ligation-dependent probe amplification (MLPA).

21. The method of claim 19, wherein the method comprises producing a plurality of amplicons from a plurality of target nucleic acids in each chromosomal region.

22. The method of claim 21, wherein the method comprises producing a plurality of amplicons from a plurality of target nucleic acids in each of one or more control chromosomal regions.

23. The method of claim 1, wherein the method comprises high-throughput DNA sequencing.

24. The method of claim 23, wherein the method comprises sequencing a plurality of target nucleic acids in each chromosomal region.

25. The method of claim 24, wherein the method comprises sequencing a plurality of target nucleic acids in each of one or more control chromosomal regions.

26-126. (canceled)
